(19) United States
(12) Patent Application Publication
(10) Pub. No.: US 2006/0004005 A1
(43) Pub. Date: Jan. 5, 2006

(54) Triazines derivatives as cell adhesion inhibitors

(76) Inventors: Viswajanani J. Sattigeri, Gurgaon (IN); Mohammad Salman, Plainsboro, NJ (US); Ramamurthi Narayanan, Varanasi (IN); Subhendu Seth, Howrah (IN); Simi Sarin, Gurgaon (IN); Abhijit Ray, New Delhi (IN); Sunanda Ghose Dastidar, New Delhi (IN)

Correspondence Address: Jayadeep R. Deshmukh, Esq.;Ranbaxy Inc., Suite 2100, 600 College Road East, Princeton, NJ, 08901, US

(21) Appl. No.: 10/949,096

(22) Filed: Sep. 24, 2004

Related U.S. Application Data
(60) Provisional application No. 60/505,886, filed on Sep. 25, 2003.

(51) Int. Cl. A61K 31/53

(52) U.S. Cl. 514/241; 544/209

(57) ABSTRACT

The present invention relates to triazine derivatives as cell adhesion inhibitors. The compounds of this invention can be useful inter alia, for inhibition and prevention of cell adhesion and cell adhesion-mediated pathologies, including inflammatory and autoimmune diseases such as bronchial asthma, rheumatoid arthritis, type I diabetes, multiple sclerosis, allograft rejection, psoriasis. The compounds can be used to formulate pharmacological compositions, and the methods of treating bronchial asthma, rheumatoid arthritis, multiple sclerosis, type I diabetes, psoriasis, allograft rejection, and other inflammatory and/or autoimmune disorders, using the compounds are also provided herein.

### FIELD OF THE INVENTION

The present invention relates to triazine derivatives as cell adhesion inhibitors. The compounds of this invention can be useful inter alia, for inhibition and prevention of cell adhesion and cell adhesion-mediated pathologies, including inflammatory and autoimmune diseases such as bronchial asthma, rheumatoid arthritis, type I diabetes, multiple sclerosis, allograft rejection, psoriasis. The compounds can be used to formulate pharmaceutical compositions, and the methods of treating bronchial asthma, rheumatoid arthritis, multiple sclerosis, type I diabetes, psoriasis, allograft rejection, and other inflammatory and/or autoimmune disorders, using the compounds are also provided herein.

### BACKGROUND OF THE INVENTION

Cell adhesion is a process by which cells associate with each other, migrate towards a specific target or localize within the extra-cellular matrix. These interactions are mediated by specialized molecules called cell adhesion molecules (CAM). CAMs have been demonstrated to participate in various cell-cell, cell-extracellular matrix, and platelet interactions. They influence the adhesion of leukocytes to the vascular endothelium, their transendothelial migration, retention at extravascular sites and activation of T cells and eosinophils. These processes are central to the pathogenesis of inflammatory and autoimmune diseases. Therefore, adhesion molecules are considered as potential targets to treat such disorders.

CAMs can be classified into three groups—integrins, selectins and the immunoglobulin superfamily. Out of these, integrins are key mediators in the adhesive interactions between hemopoietic cells and their microenvironment. They include alpha-beta heterodimers and integrate signals from outside of the cells to inside and vice versa. Integrins can be classified on the basis of the alpha and beta subunits they contain. For example, the beta-1 subfamily contains beta-1 subunit non-covalently linked to one of the 10 different alpha subunits.

The alpha-4 beta-1 integrin, also known as VLA₄ (very late activation antigen 4), is a member of beta 1 integrin family and consists of alpha-4 and beta-1 subunits. VLA₄ interacts with two specific ligands—the vascular cell adhesion molecule (VCAM-1) and the the CS1 region of fibronectin. Adhesion mediated by VLA₄ is central to the process of transendothelial migration of leukocytes. Ligation of VLA₄ is followed by gross rearrangement of the cytoskeleton leading to flattening of cells along the blood vessel wall followed by expression of specific molecules which digest the endothelial cell wall and diapedesis. Once in the extraluminal region, the interactions of VLA₄ with extracellular fibronectin play a crucial role in migration to the site of inflammation, T cell proliferation, expression of cytokines and inflammatory mediators. In addition, VLA₄ ligation provides costimulatory signal to the leukocytes resulting in enhanced immunoreactivity. Therefore, it is expected that VLA₄ antagonists would ameliorate the immune response through twofold actions—inhibition of T cell recruitment at the site of inflammation and inhibition of costimulatory activation of immune cells.

In this respect, inhibitors of VLA₄ interactions have demonstrated beneficial therapeutic effects in several animal models of inflammatory, and allergic diseases including sheep allergic asthma, experimental allergic encephomyelitis, contact hypersensitivity and inflammatory bowel.

Region of CS1 moiety of fibronectin involved in the interaction with VLA₄ was identified as the tripeptide Leu-Asp-Val. also known as LDV. Taking a lead from this, several peptides containing the LDV sequence were synthesised which have shown to inhibit the in vivo interaction of VLA₄ to its ligands.

Despite these advances, there remains a need for small and specific inhibitors of VLA₄ dependent cell adhesion molecules. Ideally such inhibitors should be water soluble with oral efficacy. Such compounds would provide useful agents for treatment, prevention or suppression of various inflammatory pathologies mediated by VLA₄ binding.

In support of this concept, inhibitors of VLA-4 interactions have demonstrated beneficial therapeutic effects in several animal models of inflammatory, and allergic diseases including sheep allergic asthma (Abraham et al, J. Clin. Invest., 93, 776 (1994)), arthritis (Wahl et al, J. Clin. Invest. 94, 655 (1994)); experimental allergic encephomyelitis (Yednock et al, Nature (Lond), 356, 63 (1992) and Baron et al, J. Exp. Med., 177, 57 (1993)); contact hypersensitivity (Chisolm et al, Eur J. Immunol., 23, 682 (1993)); type I diabetes (Yang et al, Proc. Natl. Acad. Sci. (USA), 90, 10494 (1993)) and inflammatory bowel disease (Podolsky et al, J. Clin. Invest., 92, 372 (1993)).

U.S. Pat. No. 6,329,344 B1 discloses monosaccharide derivatives as cell adhesion inhibitors. It generally relates to a group of substituted pentose and hexose monosaccharide derivatives, which exhibit potent anti-cell adhesion and anti-inflammatory activities. PCT application WO 00/42054 discloses several monosaccharide derivatives as cell adhesion inhibitors.

U.S. patent application 2002/0055509 A1 discloses a series of phenylalanine derivatives which are potent and selective inhibitors of α₄ integrins. They employ various heterocycles as derivatives, including substituted diazines, pyrrolyls, furyls, triazolyls, triazinyls, imidyls and other heterocyclic groups.

Patent application WO 00/43369 provides compounds which bind to VLA-4, and also relates to triazine derivatives which inhibit leukocyte adhesion mediated by VLA-4.

Bioorganic and Medicinal Chemistry Letters 12 (2002) 1591-1594 relates to discovery and evaluation of N-(triazin-1,3,5-yl) phenylalanine derivatives as VLA-4 integrin antagonists in which SAR studies aimed at improving the rate of clearance of a series of VLA-4 integrin antagonists by the introduction of a 1,3,5-triazine as an amide isostere are described.

Bioorganic and Medicinal Chemistry Letters 12 (2002) 1595-1598 relates to N-(pyrimidin-4-yl) and N-(pyridin-2-yl) phenylalanine derivatives as VLA-4 integrin antagonists in which SAR studies to optimize both potency and rate of clearance in the rat for a series of pyrimidine and pyridine based VLA-4 integrin antagonists are described.

This patent application discloses compounds containing triazine moiety coupled with urea or its bioisosteric analogues which may be used as therapy for the inhibition, prevention and suppression of VLA-4 mediated cell adhesion and the treatment of pathologies associated with that adhesion.

The compounds of the present invention may be screened for inhibitory activity in VLA-4 mediated cell adhesion assay and the classical murine hypersensitivity assay in mice. These compounds could be used in treatment of chronic, cell adhesion mediated, allergic, autoimmune and inflammatory disorders, such as bronchial asthma and rheumatoid arthritis. Some of the prior art describes development of peptide derivatives as cell adhesion antagonists for treatment of these diseases. However, because treatment of chronic diseases requires prolonged (mid term to long term) administration of drugs, development of small molecule, specific, orally available inhibitors of cell adhesion would be very beneficial.

There is no disclosure in the prior art wherein the compounds described herein, containing a triazine nucleus coupled with a urea or its bioisosteric analogues, are used as therapy for inhibition, prevention, and suppression of VLA₄-mediated cell adhesion and the treatment of pathologies associated with that adhesion.

### SUMMARY OF THE INVENTION

Herein is provided a new class of compounds that exhibit significant activity as VLA-4 antagonist, and these triazine-based molecules exhibit potential anti-inflammatory activity.

The introduction of a urea moiety, or bioisosteric analogues at various positions of the triazine nucleus introduces VLA-4 antagonism activity.

In one particular aspect there is provided a compound having the structure of Formula I: 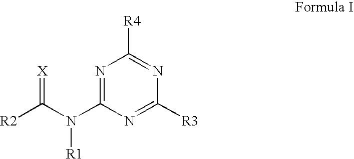
its pharmaceutically acceptable salts, pharmaceutically acceptable solvates, esters, enantiomers, diastereomers, polymorphs, N-oxides or metabolites
wherein

In Formula I,

R₁ can represent, hydrogen, alkyl, alkenyl, alkynyl, cycloalkyl, aralkyl, heteroaryl, heterocyclyl, heteroarylalkyl, or heterocyclylalkyl.

R₂ can represent, alkyl, cycloalkyl, aryl, aralkyl, heteroaryl, heterocyclyl, heteroarylalkyl, heterocyclylalkyl, or —NR₅R₆ (wherein R₅ and R₆ are independently selected from hydrogen, alkyl, alkenyl, alkynyl, cycloalkyl, aryl, aralkyl, or SO₂R₁₂ [wherein R₁₂ is selected from the group alkyl, aryl, or heteroaryl], C(═O)R^{z} [wherein R^{z} is selected from the group alkyl, aralkyl, heteroarylalkyl, aryl, heteroaryl, or heterocyclyl], or C(═O)OR^{z} wherein R^{z} is the same as defined above, heterocyclyl, heteroaryl, heteroarylalkyl, heterocyclylalkyl).

X can represent oxygen atom, -sulphur atom, N(CN), N(NO₂) or CH(NO₂), provided that X is N(CN), N(NO₂) or CH(NO₂) when R₂ is NR₅R₆.

R₃ can represent halogen (F, Cl, Br and I), alkyl, alkenyl, alkynyl, cycloalkyl, hydroxyl, alkoxy, aryl, aralkyl, aryloxy, heterocyclyl, heteroaryl, heteroarylalkyl, heterocyclylalkyl, or NR₇R₈ (wherein R₇ and R₈ are independently selected from hydrogen, alkyl, alkenyl, alkynyl, cycloalkyl, aryl, aralkyl, biaryl, SO₂R₁₂ [wherein R₁₂ is selected from the group alkyl, aryl or heteroaryl], C(═O)R^{z} [wherein R^{z} is selected from the group alkyl, aralkyl, heteroarylalkyl, aryl, heteroaryl, or terocyclyl], C(═O)OR^{z} wherein R^{z} is the same as defined above, heteroaryl, heterocyclyl, heteroarylalkyl, or heterocyclylalkyl).

R₄ can represent hydrogen, alkyl, alkenyl, alkynyl, aryl, aralkyl, halogen (F, Cl, Br and I), alkoxy, CH(CO₂R)₂ (wherein R represents hydrogen, alkyl, or aralkyl), heteroaryl, heterocyclyl, heteroarylalkyl, heterocyclylalkyl, NR₉R₁₀ (wherein R₉ and R₁₀ are independently selected from the group hydrogen, lower (C₁-C₆)alkyl, [further 1-2 hydrogens in the lower alkyl(C₁-C₆) may be substituted with (CH₂)ₜCO₂R₁₁ {wherein t is an integer in the range of 0-6 and R₁₁ is selected from hydrogen, alkyl, alkenyl, alkynyl, aralkyl, metal ions (Na⁺, K⁺, Ca²⁺ or Mg²⁺), ammonia, alkylated ammonia derivatives such as tris hydroxymethylaminomethyl}, aryl, aralkyl, heterocyclyl, heteroaryl, heteroarylalkyl, or heterocyclylalkyl], SO₂R₁₂ [wherein R₁₂ is selected from the group alkyl, aryl or heteroaryl], C(═O)R^{z} [wherein R^{z} is selected from the group alkyl, aralkyl, heteroarylalkyl, aryl, heteroaryl, or heterocyclyl], C(═O)OR^{z} wherein R^{z} is the same as defined above —(CH₂)mCO2R11 [wherein m is an integer in the range varying from 1 to 6, R₁₁ is the same as defined earlier and further one hydrogen of CH₂ of —(CH₂)ₘCO₂R₁₁ may be substituted with heteroaryl or heterocyclyl], aryl, aralkyl, heteroaryl, heterocyclyl, heteroarylalkyl, or heterocyclylalkyl).

The following definitions apply to terms as used herein.

The term “alkyl” unless and otherwise specified refers to a monoradical branched or unbranched saturated hydrocarbon chain having from 1 to 20 carbon atoms. It may further be substituted with one or more substituents selected from alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, thiol, alkylthio, aryloxy, aminosulfonyl, aminocarbonylamino, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all 1-3 substituents chosen from alkyl, carboxy, carboxy-alkyl, aminocarbonyl, hydroxy, alkoxy, halogen, CF₃, amino, substituted amino, cyano, and —S(O)ₙR₁₂, where R₁₂ is alkyl, aryl, or heteroaryl and n is 0, 1 or 2; or an alkyl group as defined above that is interrupted by 1-5 atoms of groups independently chosen from oxygen, sulfur and —NRₐ—, where Rₐ is chosen from hydrogen, alkyl, cycloalkyl, alkenyl, cycloalkenyl, alkynyl, aryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR₁₂, where n and R₁₂ are the same as defined earlier; or an alkyl group as defined above that has both substituents as defined above and is also interrupted by 1-5 atoms or groups as defined above.

The term “alkenyl” refers to a monoradical of a branched or unbranched unsaturated hydrocarbon group preferably having from 2 to 20 carbon atoms with cis or trans geometry either in a acyclic or cyclic ring system wherein the cyclic may include mono- or multicyclic ring forms. In the event that alkenyl is attached to the heteroatom, the double bond cannot be alpha to the heteroatom. It may further be substituted with one or more substituents selected from alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, thiol, alkylthio, ary, aryloxy, aminosulfonyl, aminocarbonylamino, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR₁₂, where R₁₂ is alkyl, aryl, or heteroaryl and n is 0, 1 or 2.

The term “alkynyl” refers to a monoradical of an unsaturated hydrocarbon, preferably having from 2 to 20 carbon atoms. In the event that alkynyl is attached to the heteroatom, the triple bond cannot be alpha to the heteroatom. It may further be substituted with one or more substituents selected from alkyl, alkenyl, alkynyl, alkoxy, cycloalkyl, cycloalkenyl, acl, acylamino, acyloxy, amino, aminocarbonyl, alkoxycarbonylamino, azido, cyano, halogen, hydroxy, keto, thiocarbonyl, carboxy, carboxyalkyl, arylthio, thiol, alkylthio, aryl, aryloxy, aminosulfonyl, aminocarbonylamino, hydroxyamino, alkoxyamino, nitro, —SO-alkyl, —SO-aryl, —SO-heteroaryl, —SO₂-alkyl, SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR₁₂, where R₁₂ is alkyl, aryl, or heteroaryl and n is 0, 1 or 2.

The term “cycloalkyl” refers to cyclic alkyl groups of from 3 to 20 carbon atoms having a single cyclic ring or multiple condensed rings. It may further be substituted with one or more substituents selected from alkyl, alkenyl, alkynyl, alkoxy, cycloalkyl, cycloalkenyl, acyl, acylamino, acyloxy, amino, aminocarbonyl, alkoxycarbonylamino, azido, cyano, halogen, hydroxy, keto, thiocarbonyl, carboxy, carboxyalkyl, arylthio, thiol, alkylthio, aryl, aryloxy, aminosulfonyl, aminocarbonylamino, hydroxyamino, alkoxyamino, nitro, —SO-alkyl, —SO-aryl, —SO-heteroaryl, —SO₂-alkyl, —SO₂-aryl and —SO₂-heteroaryl. Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR₁₂, where R₁₂ is alkyl, aryl or heteroaryl and n is 0, 1 or 2.

“Alkoxy” denotes the group O-alkyl wherein alkyl is the same as defined above.

“Aralkyl” refers to (CH₂)ₚ aryl, wherein p is an integer in the range of 1-6 and aryl is as defined below.

“Aryl” refers to an optionally substituted phenyl or naphthyl ring wherein the substituents may be selected from alkyl, alkenyl, alkynyl, cycloalkyl, cycloalkenyl, halogen (F, Cl, Br and I), hydroxyl, alkoxy, aryl, nitro, amino or substituted amino, aminothiocarbonyl, alkoxycarbonyl, cyano, azido, oxo, thiocarbonyl, thiol, SOₙR₁₂ (wherein n is an integer in the range of 0-2 and R₁₂ is selected from the group alkyl, aryl, or heteroaryl), aminosulfonyl, aminocarbonylamino, or C(═O)R^{x} wherein R^{x} is selected from the group of hydrogen, alkyl, aryl, aralkyl, hydroxyl, alkoxy, amino or substituted amino)

“Aryloxy” denotes the group O-Aryl wherein aryl is the same as defined above.

“Substituted amino” refers to a group —N(Rᵣ)₂ wherein each Rᵣ can be independently selected from Hydrogen, provided that both Rr groups are not hydrogen (defined as “amino”) alkyl, alkenyl, alkynyl, aralkyl, cycloalkyl, aryl, heteroaryl, heterocyclyl, heterocyclylalkyl, heteroarylalkyl, SO₂R₁₂ wherein R₁₂ is selected from the group alkyl, aryl or heteroaryl), C(═O)R^{z} (wherein R^{z} is selected from the group alkyl, aralkyl, heteroarylalkyl, aryl, heteroaryl, or heterocyclyl), C(═O)OR^{z} wherein R^{z} is the same as defined above, or —(CH₂)ₘCO₂R₁₁, wherein m is an integer in the range varying from 1 to 6 and R₁₁ is the same as defined earlier). Unless otherwise constrained by the definition, all substituents may optionally be further substituted by 1-3 substituents chosen from alkyl, carboxy, carboxyalkyl, aminocarbonyl, hydroxy, alkoxy, halogen, CF₃, amino, substituted amino, cyano, and —S(O)ₙR₁₂, where R₁₂ is alkyl, aryl or heteroaryl and n is 0, 1 or 2.

“Heteroaryl” refers to a monocyclic or bicyclic aromatic ring structure containing one to four heteroatom(s) selected from N, O and S, it may optionally be substituted with one or more of substituents selected from alkyl, alkenyl, alkynyl, halogen (F, Cl, Br and I), hydroxyl, alkoxy, nitro, amino or substituted amino, cyano, oxo, C(═O)R^{x} wherein R^{x} is the same as defined earlier, SOₙR₁₂ wherein n and R₁₂ are the same as defined earlier, or further it may optionally be fused to an aryl ring wherein aryl is the same as defined earlier.

“Heterocyclyl” refers to a saturated or unsaturated carbocyclic group having a single ring or multiple condensed rings which may be bridged or unbridged having at least one heteroatom selected from N, O and S within the ring, it may optionally be substituted with one or more of substituents selected from alkyl, alkenyl, alkynyl, halogen (F, Cl, Br and I), hydroxyl, alkoxy, aryl, aralkyl, nitro, amino or substituted amino, cyano, oxo, C(═O)R^{x} wherein R^{x} is the same as defined earlier, SOₙR₁₂ wherein n and R₁₂ are the same as defined earlier, or further it may optionally be fused to an aryl ring wherein aryl is the same as defined earlier. When the heteroatom is nitrogen, the nitrogen may be suitably substituted with hydrogen, alkyl, aryl, aralkyl, heteroaryl, SO₂R₁₂ wherein R₁₂ is the same as defined earlier, C(═O)OR^{y} (wherein R^{y} is selected from the group of, alkyl, alkenyl or aralkyl), and further the heterocyclyl ring may also be optionally fused to an aryl ring wherein aryl is the same as defined earlier.

“Heteroarylalkyl” refers to alkyl-heteroaryl group wherein the alkyl and heteroaryl are the same as defined earlier.

“Heterocyclylalkyl” refers to alkyl-heterocyclyl group wherein the alkyl and heterocyclyl are the same as defined earlier.

In accordance with a second aspect of the present invention, there is provided a list of compounds as shown below.

| Com- | |
|---|---|
| pound | |
| No. | Chemical Name |
| 1. | 1-[4-Chloro-6-(napthalen-2-yloxy)-[1,3,5]triazin-2-yl]-3-(2- |
| | methoxyphenyl)urea |
| 2. | 1-[4-Chloro-6-(napthalen-2-yloxy)-[1,3,5]triazin-2-yl]-3-(2- |
| | chlorophenyl)urea |
| 3. | 1-[4-Chloro-6-(napthalen-2-yloxy-[1,3,5]triazin-2-yl]-3-[4- |
| | chlorophenyl)urea |
| 4. | 1-[4-Choro-6-(napthalen-2-yloxy)-[1,3,5]triazin-2-yl]-3-[4- |
| | methoxyphenyl)urea |
| 5. | 2-[4-[3-(2-Methoxyphenyl)ureido]-6-(napthalen-2-yloxy)- |
| | [1,3,5]triazin-2-yl]malonic acid-di-tert-butyl ester |
| 6. | 1-[4-Methoxy-6-(naphthalen-2-yloxy]-[1,3,5]triazin-2-yl]-3-(2- |
| | methoxyphenyl)urea |
| 7. | 4-[3-(2-Methoxyphenyl)ureido]-6-naphthalen-2-yloxy)- |
| | [1,3,5]triazin-2-yl]aminoacetic acid |
| 8. | 4-[3-(2-Chlorophenyl)ureido]-6(naphthalen-2-yloxy)- |
| | [1,3,5]triazin-2-yl]aminoacetic acid |
| 9. | 4-[3-(2-Methylphenyl)ureido]-6-(naphthalen-2-yloxy)- |
| | [1,3,5]triazin-2-yl]aminoacetic acid |
| 10. | 4-[3-(4-Methoxyphenyl)ureido]-6-(naphthalen-2-yloxy)- |
| | [1,3,5]triazin-2-yl]aminoacetic acid |
| 11. | 4-[3-(4-Chlorophenyl)ureido]-6-(naphthalen-2-yloxy]- |
| | [1,3,5]triazin-2-yl]aminoacetic acid |
| 12. | 4-[3-(4-Methylphenyl)ureido]-6-(naphthalen-2-yloxy]- |
| | [1,3,5]triazin-2-yl]aminoacetic acid |
| 13. | 2S-[4-[3-(2-Methoxyphenyl)-ureido]-6-(naphthalen-2-yloxy)- |
| | [1,3,5]triazin-2-ylamino}-3-phenylpropionic acid |
| 14. | 3-{4-[3-(2-Methoxyphenyl)ureido]-6-(napthalen-2-yloxy)- |
| | [1,3,5]triazin-2-ylamino}3-phenylpropoionic acid |
| 15. | 3-{Benzo[1,3]dioxo-5-yl-[4-[3-(2-methoxyphenyl) ureido]-6- |
| | napthalen-2-yloxy)-[1,3,5]triazin-2-ylamino) propionic acid |
| | ethyl ester |
| 16. | 3-(Benzo[1,3]dioxo-5-yl-([4-[3-(2-methoxyphenyl)-ureido]-6- |
| | napthalen-2-yloxy)-[1,3,5]triazin-2-ylamino} propionic acid |
| 17. | 1-[4-(Biphenyl-2-ylamino)-[1,3,5]triazin-2-yl-3-(2- |
| | methoxyphenyl)urea |
| 18. | 1-[4-(Biphenyl-2-ylamino)-[1,3,5]triazin-2-yl}-3-(2- |
| | methoxyphenyl)urea |
| 19. | 1-[4-(Biphenyl-2-ylamino)-6-methoxy-[1,3,5]triazin-2-yl}-3-(2- |
| | methoxyphenyl)urea |
| 20. | {4-Biphenyl-2-ylamino)-6-[3-(2-methoxyphenyl)ureido]- |
| | [1.3.5]triazin-2-ylamino]acetic acid |
| 21. | 2S-{4-(Biphenyl-2-ylamino)-6-[3-(2-methoxyphenyl) ureido]- |
| | [1,3,5]triazin-2-ylamino}-3-phenylpropionic acid |
| 22. | 3-{4-Biphenyl-2-ylamino)-6-[3-(2-methoxyphenyl) ureido]- |
| | [1,3,5]triazin-2-ylamino}-3-phenylpropionic acid |
| 23. | 3-{3-Benzo[1,3]dioxo-5-yl-{4-(biphenyl-2-ylamino)-6-[3-(2- |
| | methoxyphenyl)ureido]-[1,3,5]triazin-2-ylamino} propionic |
| | acid |
| 24. | 2S-[4-(Biphenyl-2-ylamino)-6-[3-(2-methoxyphenyl) ureido]- |
| | [1,3,5]triazin-2-ylamino]-4-methylpentanoic acid |
| 25. | 2-{4-(Biphenyl-2-ylamino)-6-[3-(2-methoxyphenyl) ureido]- |
| | [1,3,5]triazin-2-ylamino}benzoic acid |
| 26. | 3-[{(Benzo[1,3]dioxo-5-ylmethyl-{4-(biphenyl-2-yl amino)-6- |
| | [3-(2-methoxyphenyl)ureido]-[1,3,5]triazin-2- |
| | yl}amino]propanoic acid |
| 27. | 3-[{4-(Biphenyl-2-ylamino)-6-[3-(2-methoxyphenyl) ureido]- |
| | [1,3,5]triazin-2-yl}-(3,4-dimethoxybenzyl) amino]-propionic |
| | acid |
| 28. | 3-[{4-(Biphenyl-2-ylamino)-6-[3-(2-methoxyphenyl) ureido]- |
| | [1,3,5]triazin-2-yl}-(4-methoxybenzyl)amino]-propionic acid |
| 29. | 3-[{4-(Biphenyl-2-ylamino)-6-[3-(2-methoxyphenyl) ureido]- |
| | [1,3,5]triazin-2-yl}-(3-methoxybenzyl)-amino] propionic acid |
| 30. | 3-[{4-(Biphenyl-2-ylamino)-6-[3-(2-methoxyphenyl)-ureido]- |
| | [1,3,5]triazin-2-yl}-(2-methoxybenzyl)amino] propionic acid |
| 31. | 3-[{4-(Biphenyl-2-ylamino]-6-[3-(2-methoxyphenyl) ureido]- |
| | [1,3,5]triazin-2-yl}-(3-methylbenzyl)amino] propionic acid |
| 32. | 3-[{4-(Biphenyl-2-ylamino)-6-[3-(2-methoxyphenyl) ureido]- |
| | [1,3,5]triazin-2-yl}-(4-methylbenzyl)amino] propionic acid |
| 33. | 3-[{4-(Biphenyl-2-ylamino)-6-[3-(2-methoxyphenyl) ureido- |
| | [1,3,5]triazin-2-yl}-(2-fluorobenzyl)amino] propionic acid |
| 34. | 3-[{4-(Biphenyl-2-ylamino]-6-[3-(2-methoxyphenyl) ureido]- |
| | [1,3,5]triazin-2-yl}-(4-fluorobenzyl)amino] propionic acid |
| 35. | 3-[{4-(Biphenyl-2-ylamino]-6-[3-(2-methoxyphenyl) ureido]- |
| | [1,3,5]triazin-2-yl}-(2-chlorophenyl)amino] propionic acid |
| 36. | 3-[{4-(Biphenyl-2-ylamino)-6-[3-(2-methoxyphenyl) ureido]- |
| | [1,3,5]triazin-2-yl}-(3,4-dichlorobenzyl)amino] propionic acid |
| 37. | 3-[{4-(Biphenyl-2-ylamino)-6-[3-(2-methoxyphenyl) ureido]- |
| | [1,3,5]triazin-2-yl}-(2,4-dichlorobenzyl)amino] propionic acid |
| 38. | 3-{4-(Biphenyl-2-ylamino]-6-[3-(2-methoxyphenyl) ureido]- |
| | [1,3,5]triazin-2-yl}amino}propionic acid |
| 39. | [{4-(Biphenyl-2-ylamino)-6-[3-(2-methoxyphenyl) ureido- |
| | [1,3,5]triazin-2-yl}-(4-methylbenzyl)amino] acetic acid |
| 40. | 3-[{4-(Biphenyl-2-ylamino]-6-[3-(2-methoxyphenyl)-ureido- |
| | [1,3,5]triazin-2-yl}-(4-methoxybenzyl)amino] acetic acid |
| 41. | [{4-(Biphenyl-2-ylamino)-6-[3-(2-methoxyphenyl) ureido]- |
| | [1,3,5]triazin-2-yl}-(3,4-dimethoxybenzyl) amino] acetic acid |
| 42. | [{4-(Benzyl-2-ylamino)-6-[3-(2-methoxyphenyl)ureido]- |
| | [1,3,5]triazin-2-yl}-[4-fluorobenzyl]amino]acetic acid |
| 43. | 3-{4-[3-(2-Methoxyphenyl)ureido]-6-(naphthalen-2-yloxy)- |
| | [1,3,5,]triazin-2-ylamino}propionic acid |
| 44. | 3-{(3-Methoxybenzyl)-[4-[3-(2-methoxyphenyl)ureido]-6- |
| | (naphthalen-2-yloxy)-[1,3,5]triazin-2-yl]amino} propionic acid |
| 45. | 3-{(4-Methoxybenzyl)-[4-[3-(2-methoxyphenyl)ureido]-6- |
| | [naphthalen-2-yloxy)-[1,3,5]triazin-2-yl]amino} propionic acid |
| 46. | 3-{(4-Fluorobenzyl)-[4-[3-(2-methoxyphenyl)-ureido]-6- |
| | [naphthalen-2-yloxy)-[1,3,5]triazin-2-yl]amino} propionic acid |
| 47. | 3-{(4-Methylbenzyl)-[4-[3-(2-methoxyphenyl)ureido]-6- |
| | [naphthalen-2-yloxy)-[1,3,5]triazin-yl]amino}propionic acid |
| 48. | Tris salt of 3-[{4-(Biphenyl-2-ylamino)-6-[3-(2- |
| | methoxyphenyl) ureido]-[1,3,5]triazin-2-yl}-(3- |
| | methylbenzyl)amino] propionic acid |
| 49. | Na salt of 3-[{4-(Biphenyl-2-ylamino)-6-[3-(2-methoxyphenyl)- |
| | ureido]-[1,3,5]triazin-2-yl}-(3-methylbenzyl)amino] propionic |
| | acid |
| 50. | 3-{Benzo[1,3]dioxo-5-ylmethyl-[4-[3-(2-methoxyphenyl)- |
| | ureido]-6-(napthanalene-2-yloxy)-[1,3,5]triazin-2- |
| | yl]amino}propionic acid |
| 51. | 3-{2-Methoxybenzyl)-[4-[3-(2-methoxyphenyl)ureido]-6- |
| | (naphthalen-2-yloxy)-[1,3,5]triazin-2-yl]amino} propionic acid |
| 52. | 3-{(3,4-Dimethoxybenzyl)-[4-[3-(2-methoxyphenyl) ureido-6- |
| | (naphthalen-2-yloxy)-[1,3,5]triazin-2-yl] amino}propionic acid |
| 53. | 3-{(3-Methylbenzyl)-[4-[3-2-(methoxyphenyl)ureido-6- |
| | (naphthalen-2-yloxy)-[1,3,5]triazin-2-yl]amino} propionic acid |
| 54. | 3-{(2-Fluorobenzyl)-[4-[3-2-(methoxyphenyl)ureido-6- |
| | (naphthalen-2-yloxy)-[1,3,5]triazin-2-yl]amino} propionic acid |
| 55. | 3-{(2-Chlorobenzyl)-[4-[3-2-methoxyphenyl)ureido-6- |
| | (naphthelen-2-yloxy)-[1,3,5]triazin-2-yl]amino] propionic acid |
| 56. | 3-{(2,4-Dichlorobenzyl)-[4-[3-2-methoxyphenyl)ureido-6- |
| | (naphthalen-2-yloxy)-[1,3,5]triazin-2-yl]amino} propionic acid |
| 57. | 3-{3,4-Dichlorobenzyl)-[4-[3-2-methoxyphenyl)ureido-6- |
| | (naphthalen-2-yloxy)-[1,3,5,]triazin-2-yl]amino} propionic acid |
| 58. | 3-[(4-[3-(2-Methoxyphenyl)ureido]-6-chloro-[1,3,5] triazin-2- |
| | yl)(3-methyl-benzyl)amino]propionic acid |
| 59. | 3-[{4-(2-methoxyphenyl)ureido)-6-chloro-[1,3,5]triazin-2-yl}- |
| | (3,4-dichlorobenzyl)amino]propionic acid |
| 60. | 3-{(1H-Indol-3-yl)-2S([4-(2-methoxypheyl)ureido]-6- |
| | (naphthalen-2-yloxy-[1,3,5]triazin-2-yl)amino]propionic acid |
| 61. | 2-{4-[3-(2-Methoxyphenyl)ureido]-6-(naphthalen-2-yloxy)- |
| | [1,3,5]triazin-2-yl}1,2,3,4-tetrahydro-isoquinoldine-3- |
| | carboxylic acid |
| 62. | 2-{(4-Fluorobenzyl)-[4-[3-(2-methoxyphenyl)ureido-6- |
| | (naphthalen-2-yloxy)-[1,3,5]triazin-2-yl]amino}-acetic acid |
| 63. | 2-{(4-Methoxybenzyl)-[4-[3-(2-methoxyphenyl)ureido-6- |
| | (naphthalen-2-yloxy)-[1,3,5]triazin-2-yl]amino}acetic acid |
| 64. | 2-{(4-Methylbenzyl)-[4-[3-(2-methoxyphenyl)ureido-6- |
| | (naphthalen-2-yloxy)-[1,3,5]triazin-2-yl]amino}acetic acid |
| 65. | 3-{3,4-Difluorobenzyl)-[4-(3-(2-methoxyphenyl)ureido-6- |
| | naphthalen-2-yloxy)-[1,3,5]triazin-2-yl]amino} propanoic acid |
| 66. | 3-{[4-[3-(2-Methoxyphenyl)ureido]-6-(naphthalen-2-yloxy)- |
| | [1,3,5]triazin-2-yl]-thiophen-2-yl-methylamino} propionic acid |
| 67. | 3-{(4-Trifluoromethylbenzyl)-[4-[3-(2-methoxyphenyl) ureido- |
| | 6-(naphthalen-2-yloxy)-[1,3,5]triazin-2-yl] amino}propanoic |
| | acid |
| 68. | 3-{[2-(1H-Indol-3-yl)ethyl]-[4-[3-(2-methoxyphenyl) ureido]-6- |
| | (naphthalen-2-yloxy)-[1,3,5]triozin-2-yl] amino]propionic acid |
| 69. | 1-[4-[2-(4-Hydroxyphenyl)ethylamino]-6-(naphthalen-2-yloxy)- |
| | [1,3,5]triazin-2-yl]-3-(2-methoxyphenyl)urea |
| 70. | 1-[4-2-(1H-Indol-3-yl)ethylamino]-6-(naphthalen-2-yloxy)- |
| | [1,3,5]triazin-2-yl]-3-(2-methoxyphenyl)urea |
| 71. | 1-(2-Methoxyphenyl)-3-[(4-(3-methylbenzyl)amino)-6- |
| | (naphthalen-2-yloxy)-[1,3,5]triazin-2-yl]urea |
| 72. | 1-[(4-(3,4-Dichlorobenzyl)amino)-6-(naphthalen-2-yloxy)- |
| | [1,3,5]triazin-2-yl]-3-(2-methoxyphenyl)urea |
| 73. | 3-{4-(Biphenyl-2-ylamino)-6-[3-(2-methoxyphenyl) ureido]- |
| | [1,3,5]triazin-2-yl}-(4-trifluoromethyl benzyl) amino]propionic |
| | acid |
| 74. | 3-[{4-(Biphenyl-2-ylamino)-6-[3-(2-methoxyphenyl) ureido]- |
| | [1,3,5]triazine-2-yl}-(thiophen-2-ylmethyl) amino]propionic |
| | acid |
| 75. | 3-[{4-(Biphenyl-2-ylamino)-6-[3-(2-methoxyphenyl) ureido]- |
| | [1,3,5]triazin-2-yl}-(3,4-diflourobenzyl)amino] propionic acid |
| 76. | 3-[{4-(Biphenyl-2-ylamino)-6-[3-(2-methoxyphenyl) ureido]- |
| | [1,3,5]trazin-2-yl}-(2-((4-hydroxyphenyl)ethyl) |
| | amino]propionic acid |
| 77. | 3-[{4-(Biphenyl-2-ylamino)-6-[3-(2-methoxyphenyl) ureido)- |
| | [1,3,5]triazin-2-yl}-[2-(1H-indol-3-yl)ethyl] amino]propionic |
| | acid |
| 78. | 4-[{4-(Biphenyl-2-ylamino)-6-[-(2-methoxyphenyl) ureido]- |
| | [1,3,5]triazin-2-yl]amino}phenylacetic acid |
| 79. | 4-[{4-{3-(2-Methoxyphenyl)ureido}-6-(naphthalen-2-yloxy)- |
| | [1,3,5]triazin-2-yl}amino]phenylacetic acid |
| 80. | 3-{(3,4-Dichlorobenzyl)-{4-isopropylamine-6-[3-(2- |
| | methoxyphenyl)ureido}[1,3,5]triazin-2-yl}amino] propionic |
| | acid |
| 81. | 3-((3,4-Dichlorobenzyl)-{4-(3-(2-methoxyphenyl) ureido)-6- |
| | (morpholin-4-yl)[1,3,5]triazin-2-yl}amino} propionic acid |
| 82. | 3-[{4-(Biphenyl-4-ylamino)-6-[3-(2-methoxyphenyl) ureido]- |
| | [1,3,5]triazin-2-yl}-(3-methylbenzyl)amino] propionic acid |
| 83. | 3-[{6-[3-(2-Methoxyphenyl)ureido]-4-oxo-4,5-dihydro- |
| | [1,3,5]triazin-2-yl}-(3-methylbenzyl)amino]propionic acid |
| 84. | 3-[{4-[(Benzo[1,3]dioxo-5-ylmethyl)amino]-6-[3-(2- |
| | methoxyphenyl)ureido]-[1,3,5]triazin-2-yl}-(3- |
| | methylbenzyl)amino]-propionic acid |
| 85. | 3-[{4-[3-(2-Methoxyphenyl)ureido]-6-[(thiophen-2-yl- |
| | methyl)amino]-[1,3,5]triazin-2-yl}-(3-methylbenzyl) |
| | amino]propionic acid |
| 86. | 3-[{4-(2,3-Dihydro-indol-1-yl)-6-[3-(2-methoxyphenl)-ureido]- |
| | [1,3,5]triazin-2-yl}-(3-methylbenzyl)amino] propionic acid |
| 87. | 3-[(4-[3-(2-Methoxyphenyl)ureido]-6-piperidin-1-yl- |
| | [1,3,5]triazin-2-yl}-(3-methyl-benzyl)amino]propionic acid |
| 88. | 3-[{4-[3-(2-Methoxyphenyl)ureido]-6-pyrrolidin-1-yl- |
| | [1,3,5]triazin-2-yl}-(3-methylbenzyl)amino]propionic acid |
| 89. | 3-[{4-[3-(2-Methoxyphenyl)ureido]-6-morpholin-4-yl- |
| | [1,3,5]triazin-2-yl}-(3-methylbenzyl)amino]propionic acid |
| 90. | 3-[{4-Allylamino-6-[3-(2-methoxyphenyl)ureido]-[1,3,5] |
| | triazin-2-yl}-(3-methylbenzyl)amino]propionic acid |
| 91. | 3-[{4-Methoxy-6-[3-(2-methoxyphenyl)ureido]-[1,3,5] triazin- |
| | 2-yl}-(3-methylbenzyl)amino]propionic acid |
| 92. | 3-[(3,4-Dichlorobenzyl)-{6-[3-(2-methoxyphenyl) ureido]-4- |
| | oxo-4,5-dihydro-[1,3,5]triazin-2-yl}amino) propionic acid |
| 93. | 3-[{4-(Biphenyl-4-ylamino)-6-[3-(2-methoxyphenyl) ureido]- |
| | [1,3,5]triazin-2-yl}-(3,4-dichlorobenzyl)amino] propionic acid |
| 94. | 3-{(3,4-Dichlorobenzyl)-[4-(3-[2-methoxyphenyl) ureido]-6- |
| | (naphthalen-2-ylamino)-[1,3,5]triazin-2-yl}amino]-propionic |
| | acid |
| 95. | 3-((3,4-Dichlorobenzyl)-{4-[4-(2-isopropoxyphenyl)-piperazin- |
| | 1-yl]-6-[3-(2-methoxyphenyl)ureido]-[1,3,5] triazin-2- |
| | yl}amino)-propionic acid |
| 96. | 3-((3,4-Dichlorobenzyl)-{4-[4-(2-methoxyphenyl) piperazin-1- |
| | yl]-6-[3-(2-methoxyphenyl)ureido]-[1,3,5] triazin-2-yl}amino] |
| | propionic acid |
| 97. | 3-[{4-[{benzo[1,3]dioxo-5-ylmethyl)amino]-6-[3-(2- |
| | methoxyphenyl)ureido]-[1,3,5]triazin-2-yl}-(3,4-dichloro- |
| | benzyl)amino]propionic acid |
| 98. | 3-((3,4-Dichlorobenzyl)-{4-[4-[3-(2-methoxyphenyl) ureido]-6- |
| | [(thiophen-2-ylmethyl)amino]-[1,3,5]triazin-2- |
| | yl}amino]propionic acid |
| 99. | 3-[(3,4-Dichlorobenzyl)-{4-(2,3-dihydro-indol-1-yl)-6-[3-(2- |
| | methoxyphenyl)ureido]-[1,3,5]triazin-2-yl}amino] propionic |
| | acid |
| 100. | 3-((3,4-Dichlorobenzyl)-{4-[3-(2-methoxyphenyl) ureido]-6- |
| | piperidin-1-yl-[1,3,5]triazin-2-yl}amino) propionic acid |
| 101. | 3-[(3,4-Dichlorobenzyl)-{4-[3-(2-methoxyphenyl) ureido]-6- |
| | pyrrolidin-1-yl-[1,3,5]triazin-2-yl}amino) propionic acid |
| 102. | 3-{[{4-Allylamino-6-[3-(2-methoxyphenyl)ureido]- |
| | [1,3,5]triazin-2-yl}-(3,4-dichlorobenzyl)-amino] propionic acid |
| 103. | 3-((3,4-Dichlorobenzyl)-{4-methoxy-6-[3-(2-methoxy phenyl)- |
| | ureido]-[1,3,5]triazin-2-yl}amino]propionic acid |
| 104. | 3-{[4-[3-(2-Methoxyphenyl)ureido]-6-(naphthalen-2-yloxy)- |
| | [1,3,5]triazin-2-yl]-methylamino}-propionic acid |
| 105. | 3-{(3-Fluorobenzyl)-[4-[3-(2-methoxyphenyl)ureido]-6- |
| | (naphthalen-2-yloxy)-[1,3,5]triazin-2-yl]amino} propionic acid |
| 106. | 3-{(3-Chlorobenzyl)-[4-[3-(2-methoxyphenyl)ureido]-6- |
| | (napthalen-2-yloxy)-[1,3,5]triazin-2-yl]amino}propionic acid |
| 107. | 3-{[4-[3-(2-Methoxyphenyl)ureido]-6-(napthalen-2-yloxy]- |
| | [1,3,5]triazin-2-yl]-(2-trifluoromethylbenzyl) amino}propionic |
| | acid |
| 108. | 3-{[4-[3-(2-Methoxyphenyl)ureido]-6-(naphthalen-2-yloxy)- |
| | [1,3,5]triazin-2-yl]-(3-trifluoromethylbenzyl) aminopropionic |
| | acid |
| 109. | 3-{(2,4-Bis-trifluoromethylbenzyl}-[4-[3-(2-methoxy |
| | phenyl)ureido]-6-(naphthalen-2-yloxy)-[1,3,5] triazin-2- |
| | yl]amino}propionic acid |
| 110. | 3-{(Biphenyl-4-ylmethyl)-[4-[3-(2-methoxyphenyl) ureido]-6- |
| | (naphthalen-2-yloxy)-[1,3,5]triazin-2-yl] amino}propionic acid |
| 111. | 3-{[4-[3-(2-Methoxyphenyl)ureido]-6-(naphthalen-2-yloxy)- |
| | [1,3,5]triazin-2-yl]-(naphthalen-1-ylmethyl) amino}-propionic |
| | acid |
| 112. | 3-[{4-(Biphenyl-2-ylamino)-6-[3-(2-hydroxyphenyl) ureido]- |
| | [1,3,5]triazin-2-yl}-(3-methylbenzyl)amino] propionic acid |
| 113. | 1-{4-[(2-Carboxyethyl)-(3,4-dichlorobenzyl)amino]-6-[3-(2- |
| | methoxyphenyl)ureido]-[1,3,5]triazin-2-yl}-pyrrolidine-2S- |
| | carboxylic acid |
| 114. | 3-[{4-(Biphenyl-2-ylamino)-6-[3-(2-methoxyphenyl) ureido]- |
| | [1,3,5]triazin-2-yl}-(3-fluorobenzyl)amino] propionic acid |
| 115. | 3-[{4-(Biphenyl-2-ylamino)-6-[3-(2-methoxyphenyl) ureido]- |
| | [1,3,5]triazin-2-yl}-(3-chlorobenzyl)amino] propionic acid |
| 116. | 3-[{4-(Biphenyl-2-ylamino)-6-[3-(2-methoxyphenyl) |
| | ureido][1,3,5]triazin-2-yl]-(2-trifluoromethylbenzyl) |
| | amino]propionic acid |
| 117. | 3-[{4-(Biphenyl-2-ylamino)-6-[3-(2-methoxyphenyl) ureido]- |
| | [1,3,5]triazin-2-yl}-(3-trifluoromethylbenzyl) amino]propionic |
| | acid |
| 118. | 3-[{4-(Biphenyl-2-ylamino)-6-[3-(2-methoxyphenyl) ureido]- |
| | [1,3,5]triazin-2-yl}-(3,5-bis-trifluoromethyl |
| | benzyl)amino]propionic acid |
| 119. | 3-({4-(Biphenyl-2-ylamino)-6-[3-(2-methoxyphenyl) ureido]- |
| | [1,3,5]triazin-2-yl}-(biphenyl-4-ylmethyl) amino]propionic acid |
| 120. | 3-({4-(Biphenyl-2-ylamino)-6-[3-(2-methoxyphenyl) ureido]- |
| | [1,3,5]triazine-2-yl}-(naphthalen-1-ylmethyl) amino]propionic |
| | acid |
| 121. | 3-[{4-(Biphenyl-2-ylamino)-6-[3-(2-methoxyphenyl) ureido]- |
| | [1,3,5]triazin-2-yl}-(2-pyridin-2-ylethyl)amino] propionic acid |
| 122. | 1-{4-(Biphenyl-2-ylamino}-6-[2-cyanoethyl)-(3-methylbenzyl)- |
| | amino]-[1,3,5]triazin-2-yl}-3-(2-methoxyphenyl)urea |
| 123. | 3-((3,4-Dichlorobenzyl)-{4-(4-hydroxypiperidinyl)-6-[3-(2- |
| | methoxyphenyl)ureido]-[1,3,5]triazin-2-yl}amino] propionic |
| | acid |
| 124. | 3-[{4-(4-Hydroxypiperidinyl-1-yl)-6-[3-(2-methoxy |
| | phenyl)ureido]-[1,3,5]triazin-2-yl}-(3-methylbenzyl)- |
| | amino]propionic acid |
| 125. | 1-{4-(Biphenyl-2-ylamino)-6-[3-(2-methoxyphenyl) ureido- |
| | [1,3,5]triazin-2-yl}-pyrrolidine-2S-carboxylic acid |
| 126. | 1-{4-(Biphenyl-2-ylamino)-6-[3-(2-methoxyphenyl) ureido]- |
| | [1,3,5]triazin-2-yl}-thiazolidine-4-carboxylic acid |
| 127. | 1-{4-(Biphenyl-2-yl-amino)-6-[3-(2-methoxyphenyl) ureido]- |
| | [1,3,5]triazin-2-yl}-piperidine-3-carboxylic acid |
| 128. | 1-{4-(Biphenyl-2-yl-amino)-6-[3-(2-methoxyphenyl) ureido]- |
| | [1,3,5]triazin-2-yl}-piperidine-4-carboxylic acid |
| 129. | 2-{4-(Biphenyl-2-yl-amino)-6-[3-(2-methoxyphenyl) ureido]- |
| | [1,3,5]triazin-2-yl}-1,2,3,4-tetrahydro-isoquinoline-3- |
| | carboxylic acid |
| 130. | 1-{4-(Biphenyl-2-ylamino)-6-[3-(2-methoxyphenyl) ureido]- |
| | [1,3,5]triazin-2-yl}piperidine-2-carboxylic acid. |

In accordance with a further aspect, there are provided methods of preventing, inhibiting or suppressing cell adhesion in an animal, the term “animal” as defined herein includes human or mammal, comprising administering to said animal compounds as described above.

In accordance with a still further aspect, there are provided methods for treating animals suffering from bronchial asthma, rheumatoid arthritis, multiple sclerosis, type 1 diabetes, psoriasis, allograft rejection and other inflammatory and/or autoimmune disorders, comprising administering to said animal compounds as described above.

In accordance with still further aspect, there is provided a method for preventing, inhibiting or suppressing cell adhesion—associated inflammation, immune or autoimmune response in an animal by administering compounds as described above.

In accordance with yet further aspect, there is provided a method for treating or preventing a disease selected from asthma, arthritis, psoriasis, transplantation rejection, multiple sclerosis, diabetes and inflammatory bowel disease in an animal, by administering compounds as described above.

In accordance with a further aspect, there are provided processes for preparing the above compounds.

In accordance with a still further aspect, there are provided pharmaceutical compositions for the above methods, comprising compounds as described above, and a pharmaceutically acceptable carrier.

Salts of the above compounds can be obtained by the addition of various bases, including TRIS [tris(hydroxymethylaminomethane)] or alkaline hydroxides, carbonates or bicarbonates, etc, and are also included in the invention.

The compounds of the present invention exhibit significant potency in terms of their activity, which was determined by in vitro VLA₄ mediated cell adhesion assay.

### DETAILED DESCRIPTION OF THE INVENTION

The compounds of the present invention may be readily prepared by methods familiar to a person of ordinary skill in the art. In addition, the compounds of the present invention may be prepared by following illustrative reaction sequences as depicted below:

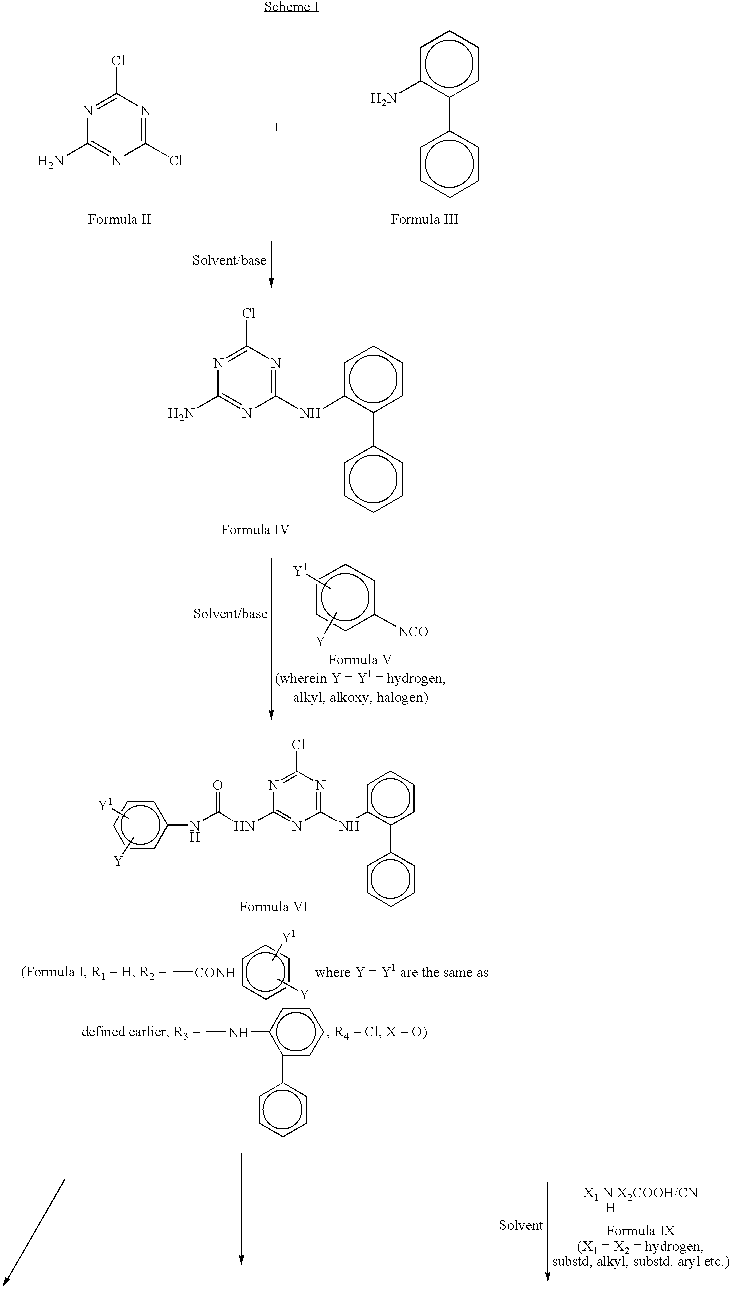

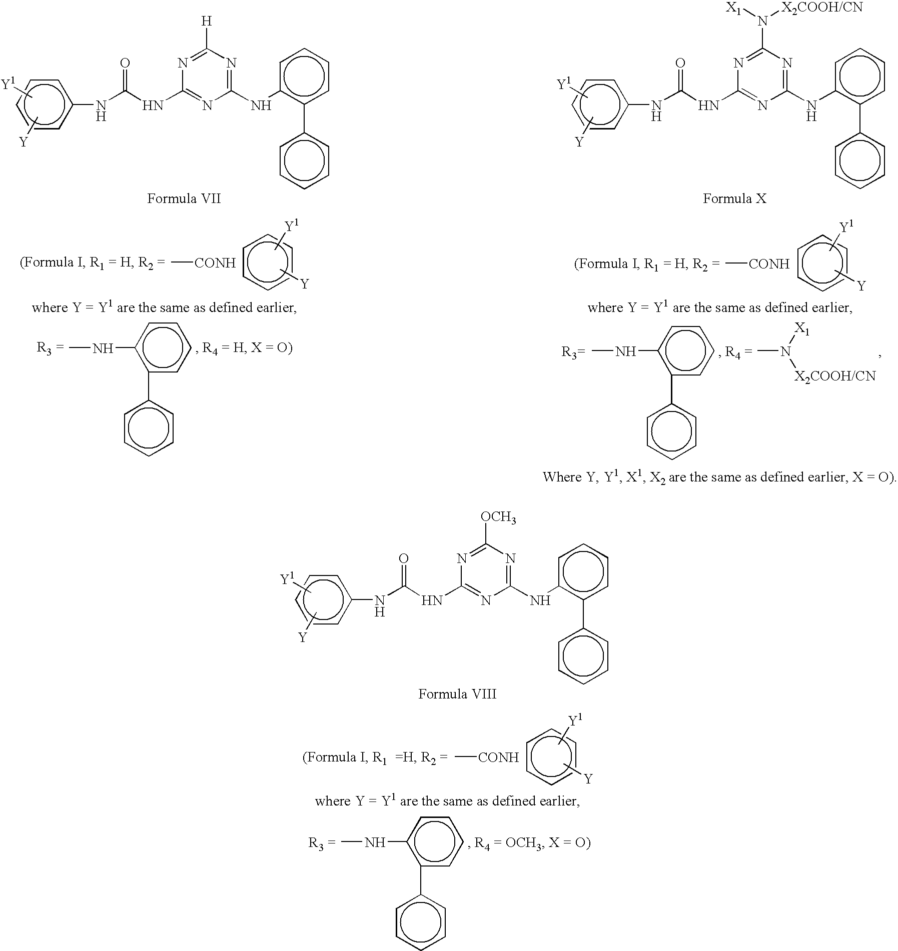

In Scheme I, 2-amino-4,6-dichloro-13,5-triazine of Formula II (prepared following the procedure described in J. Chem. Soc., 1960, 4525), was reacted with biphenylamine in a suitable solvent and in presence of a suitable base to give the compound of Formula IV which on reaction with the isocyanate of Formula V gave the compound of Formula VI (Formula I, when 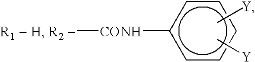
where
Y=Y¹ is hydrogen, alkyl, alkoxy, or halogen, 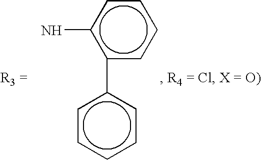
   which on reaction with substituted amino alkyl carboxylic acid in a suitable solvent gave the compound of Formula X (Formula I, when 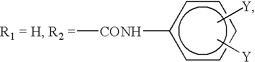
   where Y=Y¹=hydrogen alkyl, alkoxy, halogen, 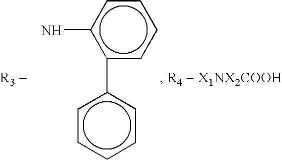
   where X₁=X₂=are the same as defined above, X═O) Further dehydrogenation of Formula VI gave the compound of Formula VII while the compound of Formula VI (Formula I, when R₁=H, R₄=H) on reaction with sodium methoxide gave the compound of Formula VIII (Formula I, when 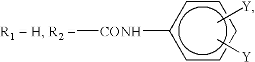
   where Y=Y¹=hydrogen, alkyl, alkoxy, halogen,

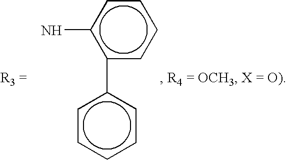

The reaction of compound of Formula II with the compound of Formula III is generally carried out in an organic solvent for example, acetone, chloroform, dichloromethane, tetrahydrofuran or dioxane.

The reaction of compound of Formula II with the compound of Formula III can be carried out in the presence of a base, for example potassium carbonate, sodium carbonate, triethylamine, diisopropylamine, pyridine or lutidine.

The reaction of compound of Formula IV with the isocyanate of Formula V is generally carried out in a solvent, for example tetrahydrofuran or dioxane. The reaction can be carried out in presence of a base, for example sodium hydride.

The reaction of compound of Formula VI with the acid compound of Formula IX is generally carried out in a solvent, for example tetrahydrofuran or dioxane to give the compound of Formula X.

The hydrogenation of compound of Formula VI to give the compound of Formula VII is generally carried out in a solvent, for example tetrahydrofuran. The reaction compound of Formula VI with sodium methoxide to give the compound of Formula VIII is generally carried out in a solvent, for example tetrahydrofuran, methanol or ethanol.

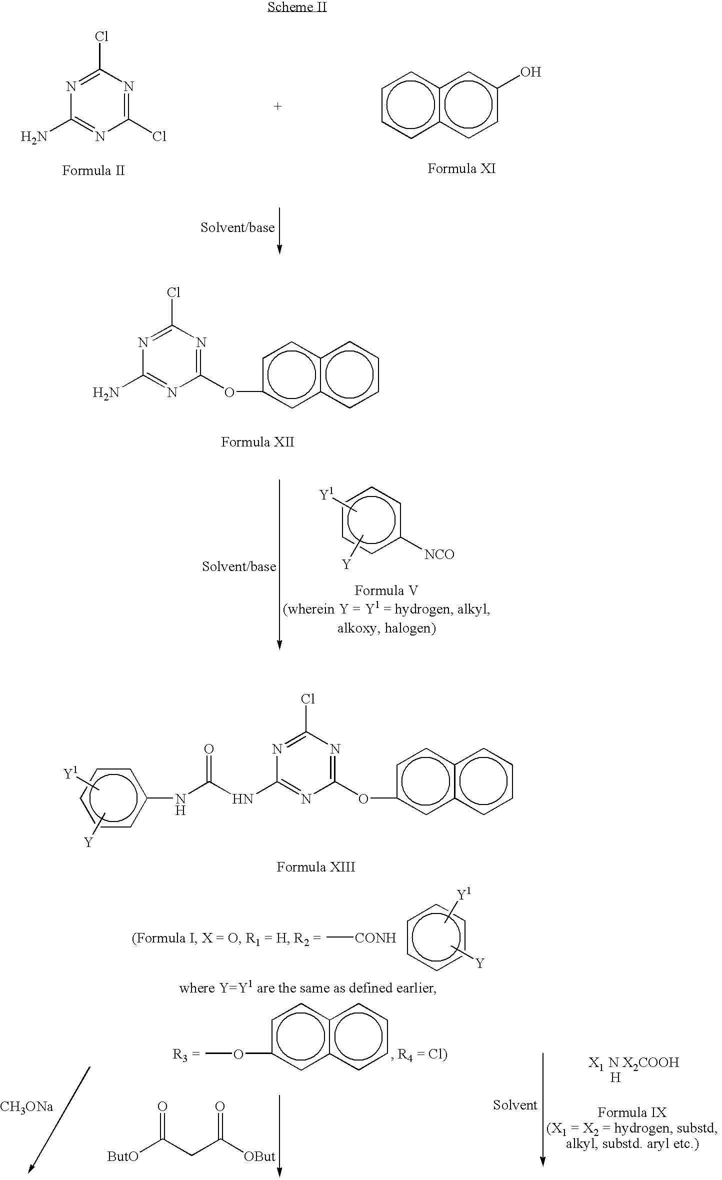

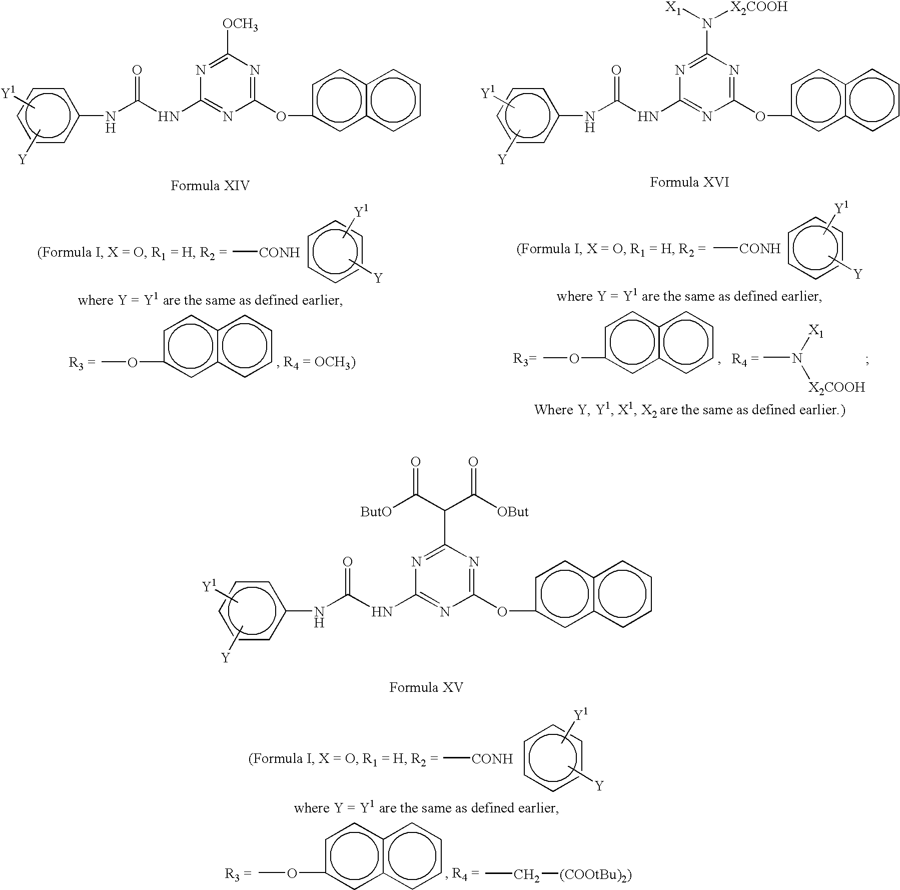

Particular compounds described herein may also be prepared by following the illustrative reaction sequence as depicted in Scheme II, where the 2-amino-4,6-dichloro-1,3,5-triazine of Formula II is reacted with β-naphthol of Formula XI in a suitable solvent and in presence of a suitable base to give the compound of Formula XII which on reaction with isocyanate of Formula V in a suitable solvent and in presence of a suitable base gave the compound of formula XIII (Formula I, 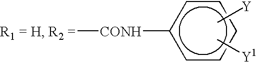
where Y, Y¹ are the same as defined earlier, 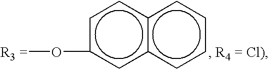
which on reaction with compound of Formula IX (where X₁, X₂=hydrogen, substd. Alkyl, substd. Aryl etc.) gave the compound of Formula XVII (Formula I, when 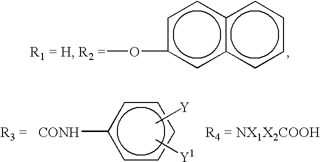
where Y, Y¹, X are the same as defined earlier, X═O). Further, the reaction of compound of Formula XIII with an alkali metal alkoxide such as sodium methoxide in presence of a base in a suitable solvent gave the compound of Formula XIV, while compound of Formula XIII on reaction with a dialkyl malonate such as dibutyl malonate gave the compound of Formula XV.

The reaction of compound of Formula II with the compound of Formula XI is generally carried out in a solvent, for example acetone, chloroform, dichloromethane, tetrahydrofuran and dioxane. The reaction can be carried out in presence of a base, such as potassium carbonate, sodium carbonate, cesium carbonate, triethylamine, lutidine or pyridine.

The reaction of compound of Formula XII with the isocyanate of Formula V is generally carried out in a solvent, for example tetrahydrofuran or dioxane, and in presence of a base, such as sodium hydride.

The reaction of compound of Formula XIII with the compound of Formula IX is generally carried out in a solvent, for example tetrahydrofuran, dioxane, water or acetone.

The reaction of compound of Formula XIII with alkoxide to give the compound of Formula XIV is generally carried out in a solvent, such as tetrahydrofuran, methanol, ethanol or butanol.

The reaction of compound of Formula XIII with malonate is generally carried out in a solvent, for example tetrahydrofuran or dioxane. The reaction can be carried out in presence of a base, such as sodium hydride, sodium methoxide or tert-butoxide.

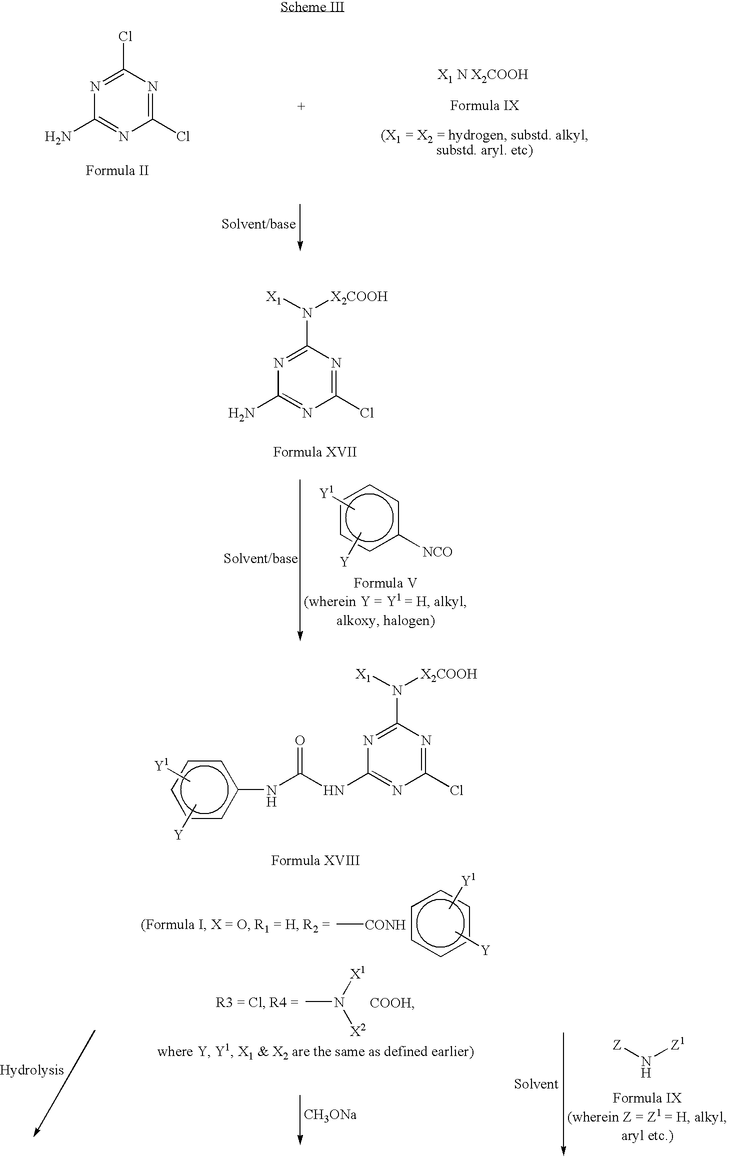

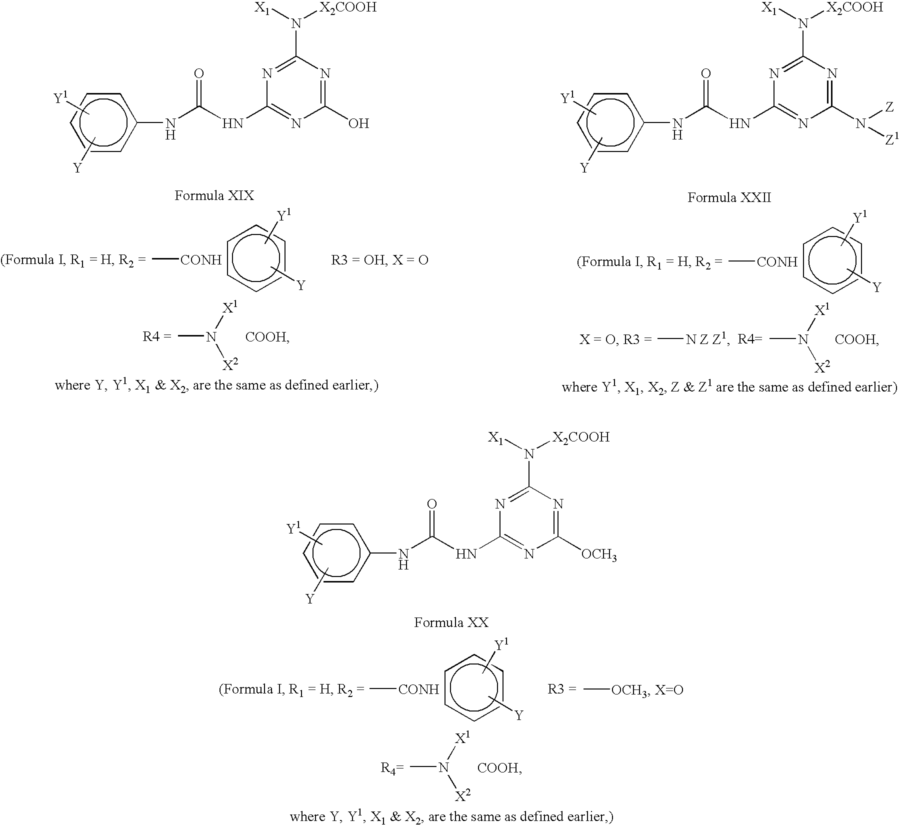

In Scheme III, the amine of Formula II is reacted with a compound of Formula IX in a suitable solvent and in presence of a suitable base to give the compound of Formula XVII which on reaction with the isocyanate of Formula V gives the compound of Formula XVIII (Formula I when 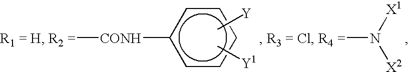
where Y, Y¹, X₁ & X₂ are the same as defined earlier, x=o) which on reaction with the compound of Formula XXI in a suitable solvent gives the compound of Formula XXII (Formula I, when 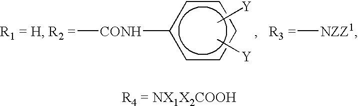
where Y, Y¹, Z, Z¹, X₁ & X₂ are the same as defined earlier, x=o). Further the compound of Formula XVIII on hydrolysis gives the compound of Formula XIX (Formula I, when 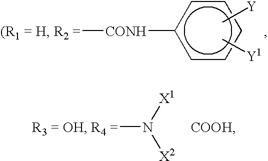
where Y, Y¹, X₁ & X₂ are the same as defined earlier, x=o) which on reaction with an alkali metal alkoxide such as sodium methoxide gives the compound of Formula XX (Formula I, when 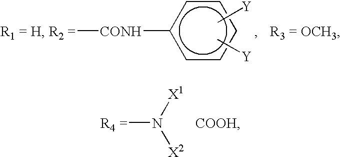
wherein Y, Y₁, X₁ and X₂ are the same as defined earlier, x=o).

The reaction of compound of Formula II with the compound of Formula IX is generally carried out in a solvent, such as acetone, chloroform, dichloromethane, tetrahydrofuran or dioxane.

The reaction of compound of Formula XVII with the isocyanate of Formula V can be carried out in presence of a base, for example sodium hydride.

The reaction of compound of Formula XVIII with compound of Formula XXI can be carried out in presence of a base, for example triethylamine, potassium carbonate, sodium hydroxide or lutidine.

The hydrolysis of compound of Formula XVIII to give the compound of Formula XIX is generally carried out in a solvent mixture, such as, for example tetrahydrofuran and t-butanol. The reaction can be carried out in presence of a strong base, for example, potassium t-butoxide.

To give the compound of Formula XX the reaction of compound of Formula XVIII with alkoxide is generally carried out in a solvent, such as tetrahydrofuran, methanol or ethanol.

The compound of general Formula I (X═S) can readily be prepared by following the illustrative procedures of any one of the above Schemes I-III by using corresponding isothiocyanate instead of isocyanates to yield the corresponding thioureas.

The compound of general Formula I (X═N(CN)) can readily be prepared by following the illustrative procedures of any one of the above Schemes I-III by treating 1 eq. of an amine/aniline with diphenyl cyanocaboimidate, followed by the addition of second amine to yield the corresponding N-cyanoguanidine derivatives.

The compound of general Formula I (X═CNO₂) can readily be prepared by following the illustrative procedures of any one of the above Schemes I-III by reacting 1 eq. of an amine/aniline with 1,1-bis(methylthio)-2-nitroethylene, further the monomethylthio derivative can be reacted with a second amine to obtain the nitroethylene derivative.

The compound of the general Formula I (R₂=lower alkyl(C₁-C₆), cycloalkyl(C₃-C₇), aryl) can readily be prepared by following the illustrative procedures of any one of the above Schemes I-III by reacting the compound of Formula II with acid chloride or coupling with an acid via methods well known in the prior art.

Particular compounds capable of being produced, for example, by Schemes 1-3 are listed below in Table 1:

TABLE 1

| | |
|---|---|
| | Formula I |
| 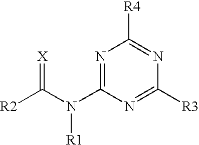 | |

| S. No. | R₁ | X | R₂ | R₃ | R₄ |
|---|---|---|---|---|---|
| 1. | H | O | 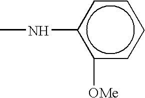 | 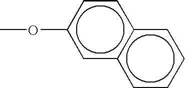 | Cl |
| 2. | H | O | 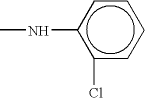 | 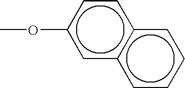 | Cl |
| 3. | H | O | 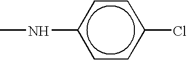 | 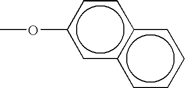 | Cl |
| 4. | H | O | 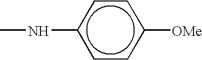 | 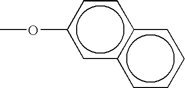 | Cl |
| 5. | H | O | 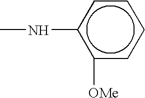 | 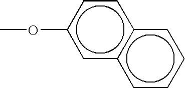 | 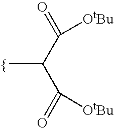 |
| 6. | H | O | 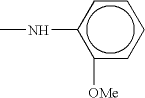 | 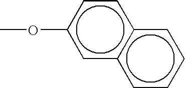 | OMe |
| 7. | H | O | 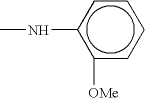 | 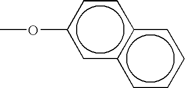 | —NHCH₂COOH |
| 8. | H | O | 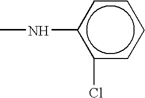 | 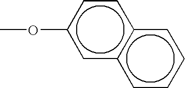 | —NHCH₂COOH |
| 9. | H | O | 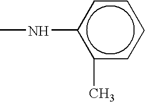 | 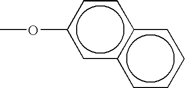 | —NHCH₂COOH |
| 10. | H | O | 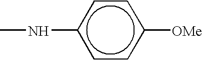 | 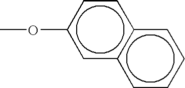 | —NHCH₂COOH |
| 11. | H | O | 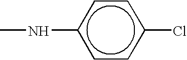 | 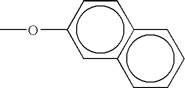 | —NHCH₂COOH |
| 12. | H | O | 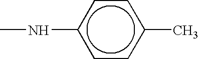 | 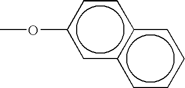 | —NHCH₂COOH |
| 13. | H | O | 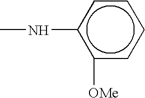 | 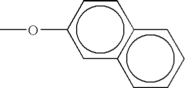 | 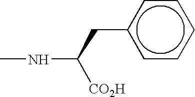 |
| 14. | H | O | 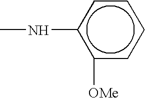 | 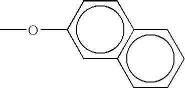 | 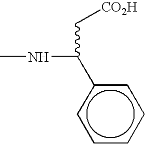 |
| 15. | H | O | 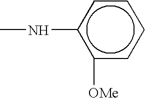 | 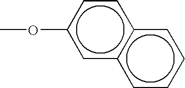 | 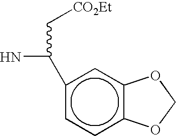 |
| 16. | H | O | 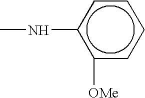 | 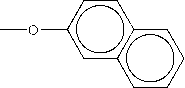 | 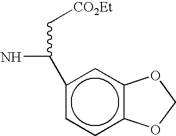 |
| 17. | H | O | 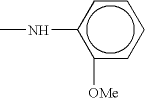 | 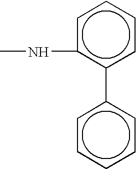 | Cl |
| 18. | H | O | 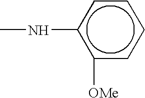 | 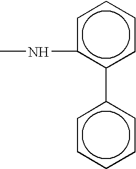 | H |
| 19. | H | O | 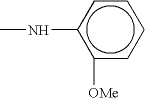 | 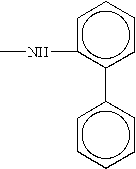 | OMe |
| 20. | H | O | 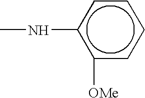 | 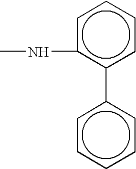 | —NHCH₂COOH |
| 21. | H | O | 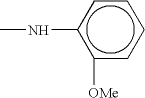 | 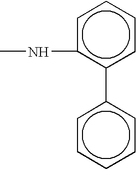 | 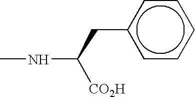 |
| 22. | H | O | 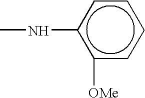 | 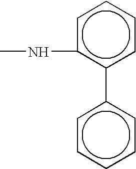 | 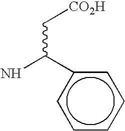 |
| 23. | H | O | 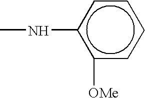 | 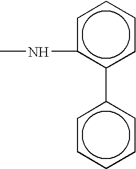 | 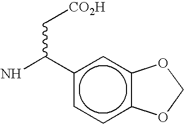 |
| 24. | H | O | 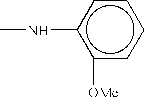 | 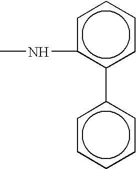 | 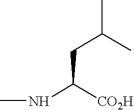 |
| 25. | H | O | 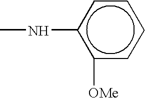 | 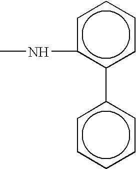 | 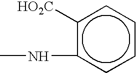 |
| 26. | H | O | 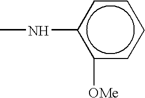 | 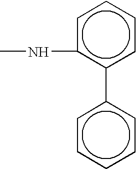 | 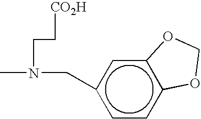 |
| 27. | H | O | 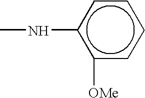 | 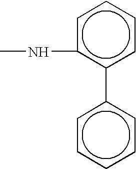 | 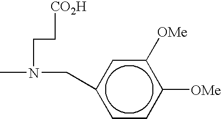 |
| 28. | H | O | 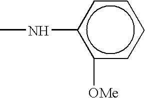 | 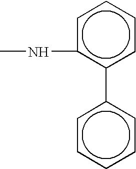 | 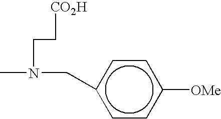 |
| 29. | H | O | 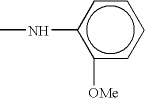 | 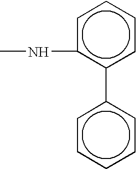 | 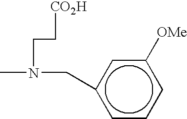 |
| 30. | H | O | 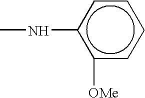 | 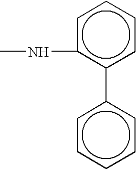 | 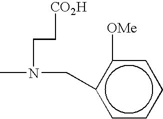 |
| 31. | H | O | 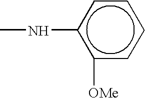 | 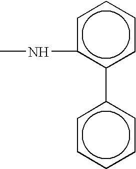 | 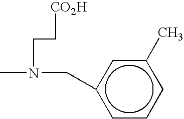 |
| 32. | H | O | 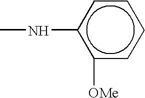 | 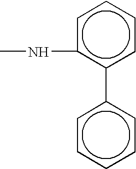 | 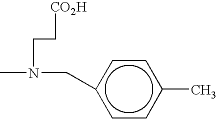 |
| 33. | H | O | 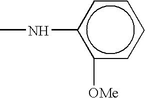 | 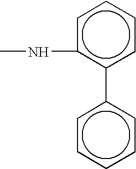 | 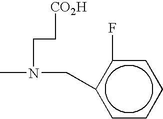 |
| 34. | H | O | 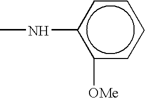 | 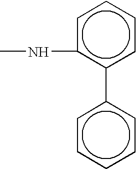 | 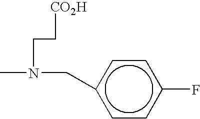 |
| 35. | H | O | 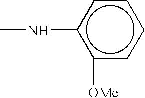 | 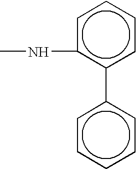 | 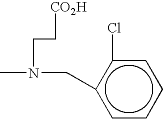 |
| 36. | H | O | 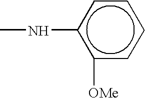 | 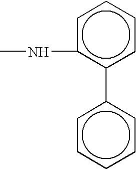 | 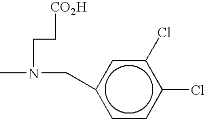 |
| 37. | H | O | 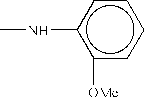 | 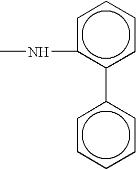 | 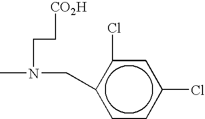 |
| 38. | H | O | 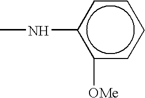 | 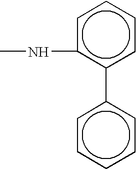 | 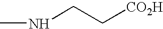 |
| 39. | H | O | 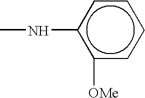 | 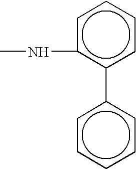 | 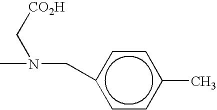 |
| 40. | H | O | 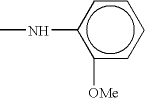 | 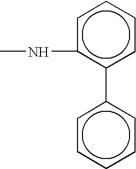 | 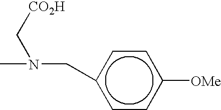 |
| 41. | H | O | 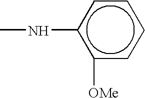 | 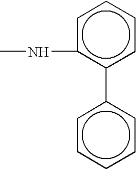 | 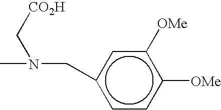 |
| 42. | H | O | 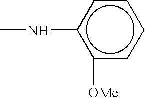 | 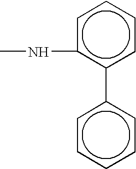 | 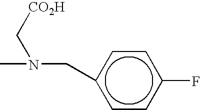 |
| 43. | H | O | 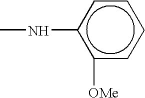 | 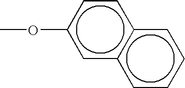 | 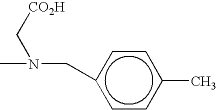 |
| 44. | H | O | 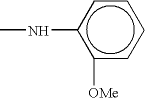 | 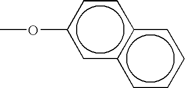 | 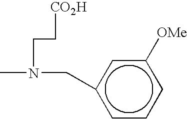 |
| 45. | H | O | 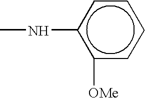 | 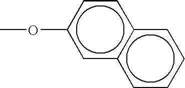 | 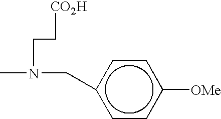 |
| 46. | H | O | 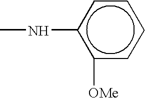 | 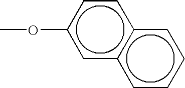 | 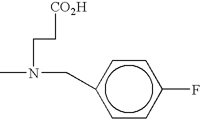 |
| 47. | H | O | 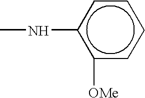 | 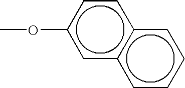 | 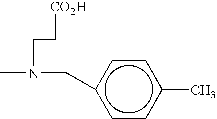 |
| 48. | H | O | 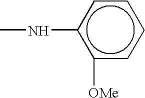 | 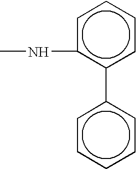 | 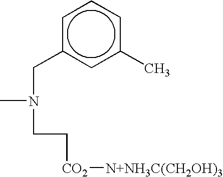 |
| 49. | H | O | 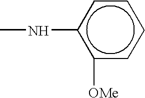 | 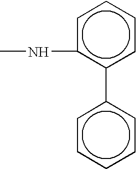 | 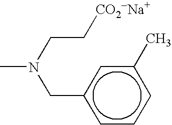 |
| 50. | H | O | 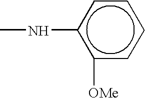 | 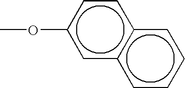 | 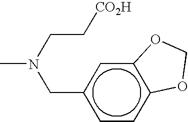 |
| 51. | H | O | 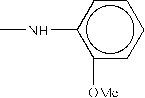 | 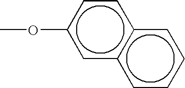 | 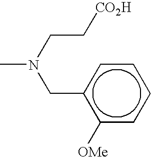 |
| 52. | H | O | 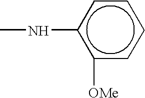 | 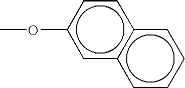 | 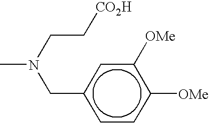 |
| 53. | H | O | 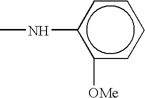 | 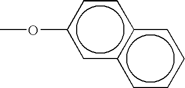 | 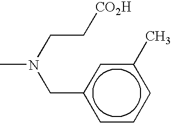 |
| 54. | H | O | 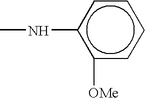 | 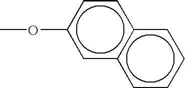 | 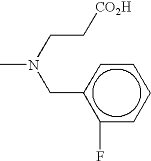 |
| 55. | H | O | 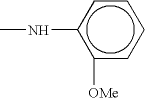 | 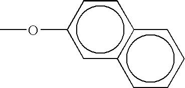 | 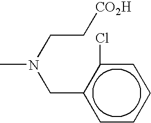 |
| 56. | H | O | 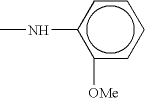 | 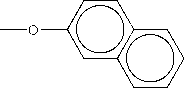 | 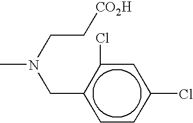 |
| 57. | H | O | 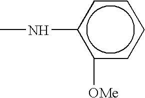 | 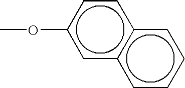 | 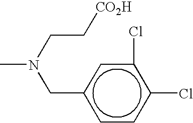 |
| 58. | H | O | 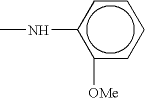 | Cl | 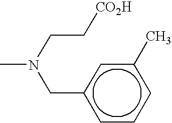 |
| 59. | H | O | 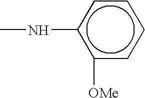 | Cl | 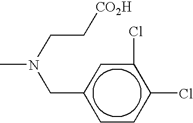 |
| 60. | H | O | 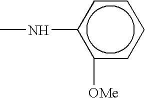 | 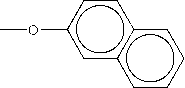 | 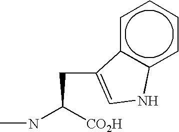 |
| 61. | H | O | 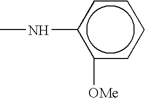 | 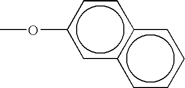 | 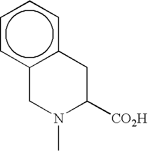 |
| 62. | H | O | 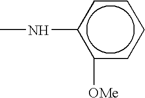 | 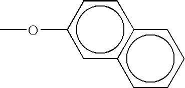 | 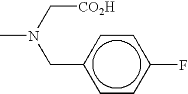 |
| 63. | H | O | 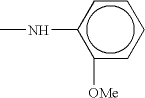 | 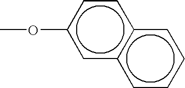 | 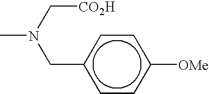 |
| 64. | H | O | 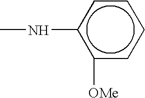 | 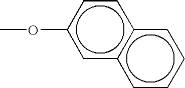 | 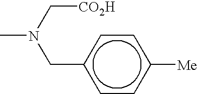 |
| 65. | H | O | 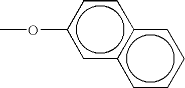 | 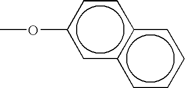 | 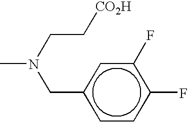 |
| 66. | H | O | 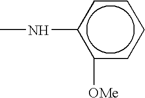 | 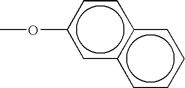 | 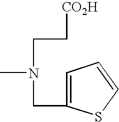 |
| 67. | H | O | 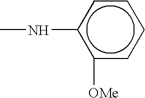 | 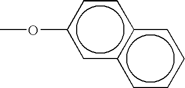 | 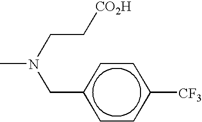 |
| 68. | H | O | 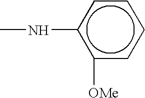 | 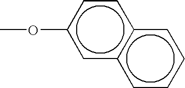 | 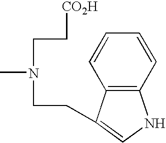 |
| 69. | H | O | 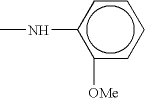 | 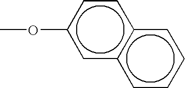 | 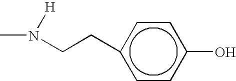 |
| 70. | H | O | 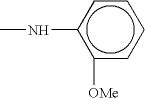 | 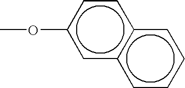 | 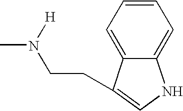 |
| 71. | H | O | 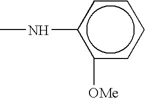 | 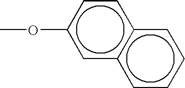 | 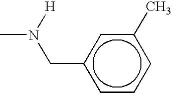 |
| 72. | H | O | 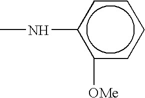 | 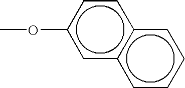 | 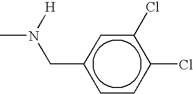 |
| 73. | H | O | 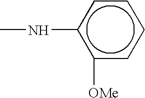 | 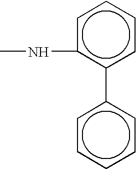 | 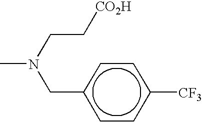 |
| 74. | H | O | 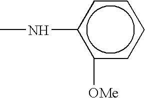 | 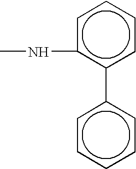 | 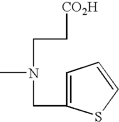 |
| 75. | H | O | 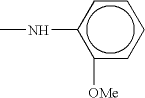 | 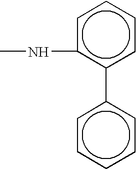 | 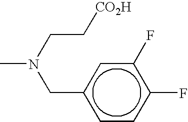 |
| 76. | H | O | 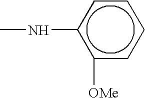 | 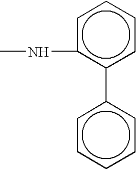 | 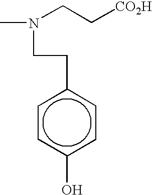 |
| 77. | H | O | 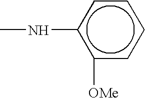 | 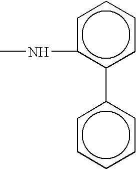 | 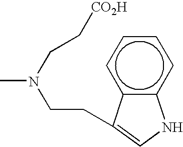 |
| 78. | H | O | 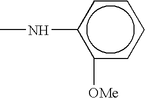 | 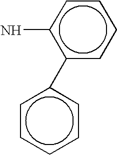 | 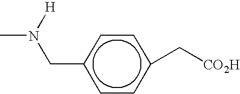 |
| 79. | H | O | 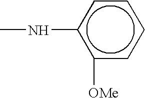 | 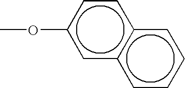 | 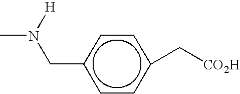 |
| 80. | H | O | 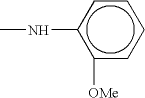 | 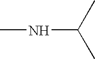 | 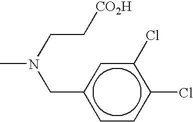 |
| 81. | H | O | 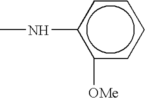 | 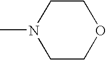 | 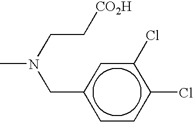 |
| 82. | H | O | 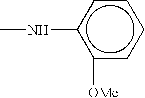 | 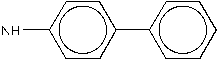 | 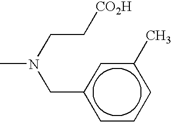 |
| 83. | H | O | 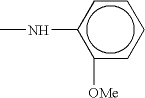 | OH | 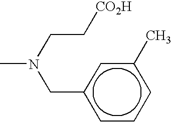 |
| 84. | H | O | 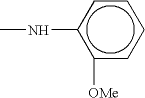 | 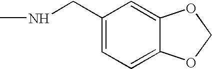 | 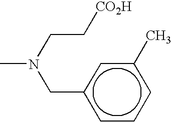 |
| 85. | H | O | 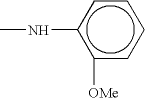 | 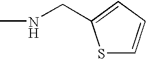 | 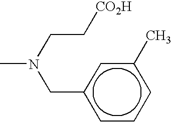 |
| 86. | H | O | 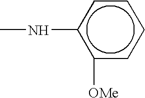 | 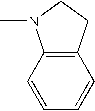 | 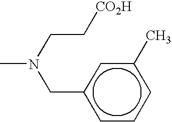 |
| 87. | H | O | 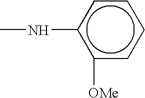 | 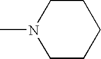 | 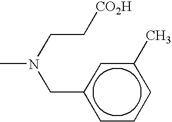 |
| 88. | H | O | 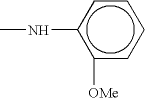 | 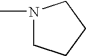 | 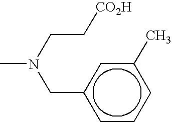 |
| 89. | H | O | 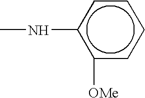 | 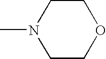 | 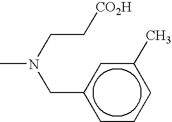 |
| 90. | H | O | 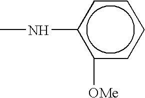 | 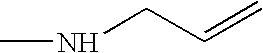 | 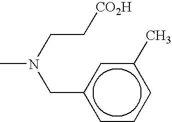 |
| 91. | H | O | 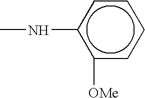 | OMe | 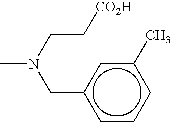 |
| 92. | H | O | 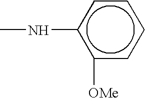 | OH | 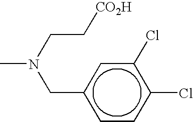 |
| 93. | H | O | 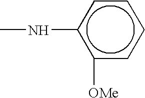 | 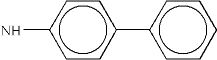 | 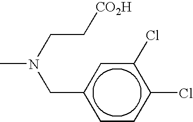 |
| 94. | H | O | 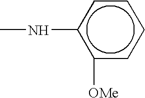 | 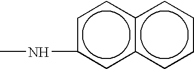 | 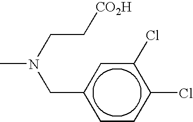 |
| 95. | H | O | 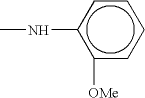 | 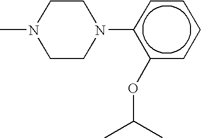 | 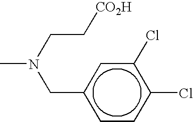 |
| 96. | H | O | 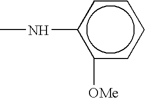 | 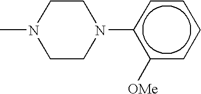 | 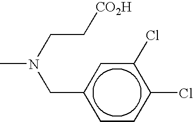 |
| 97. | H | O | 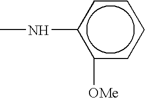 | 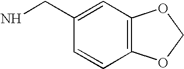 | 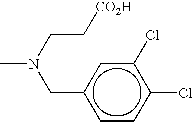 |
| 98. | H | O | 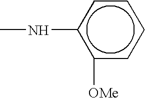 | 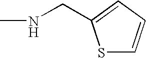 | 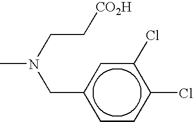 |
| 99. | H | O | 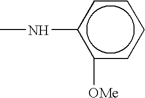 | 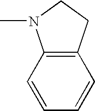 | 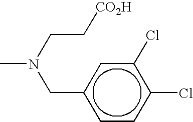 |
| 100. | H | O | 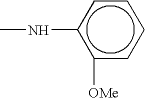 | 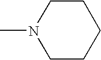 | 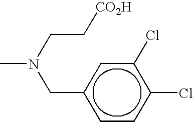 |
| 101. | H | O | 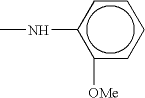 | 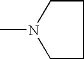 | 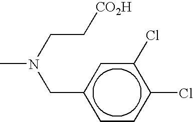 |
| 102. | H | O | 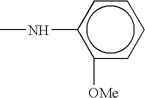 | 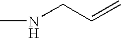 | 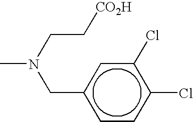 |
| 103. | H | O | 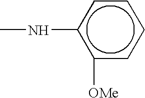 | OMe | 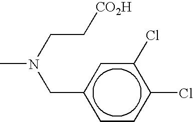 |
| 104. | H | O | 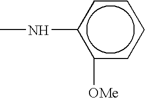 | 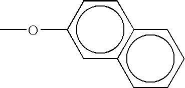 | 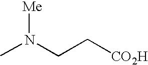 |
| 105. | H | O | 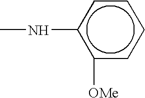 | 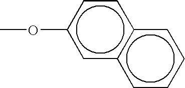 | 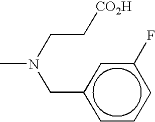 |
| 106. | H | O | 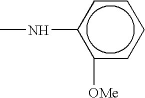 | 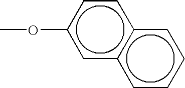 | 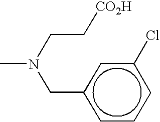 |
| 107. | H | O | 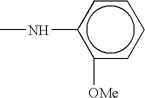 | 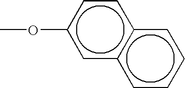 | 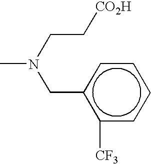 |
| 108. | H | O | 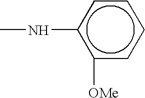 | 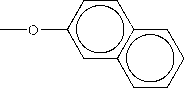 | 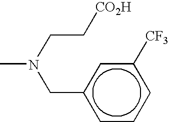 |
| 109. | H | O | 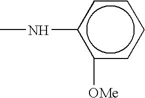 | 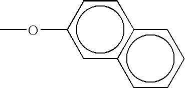 | 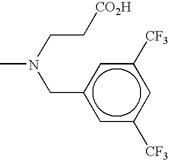 |
| 110. | H | O | 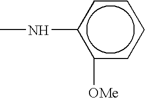 | 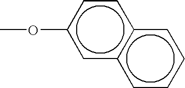 | 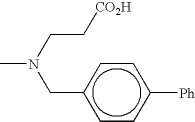 |
| 111. | H | O | 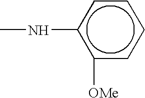 | 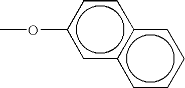 | 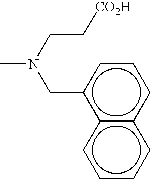 |
| 112. | H | O | 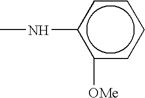 | 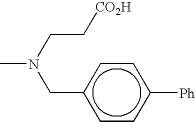 | 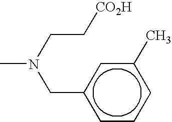 |
| 113. | H | O | 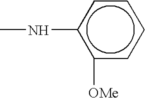 | 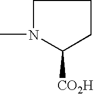 | 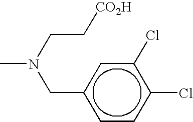 |
| 114. | H | O | 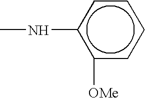 | 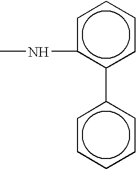 | 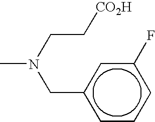 |
| 115 | H | O | 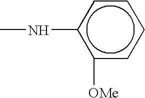 | 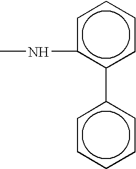 | 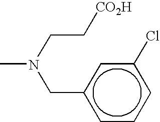 |
| 116 | H | O | 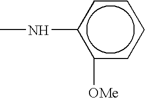 | 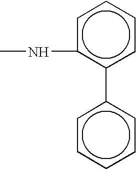 | 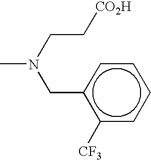 |
| 117 | H | O | 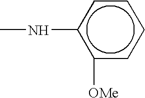 | 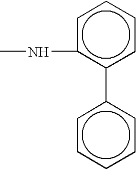 | 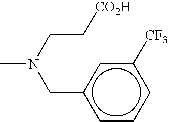 |
| 118 | H | O | 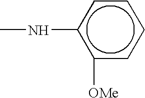 | 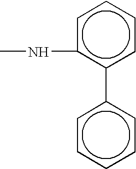 | 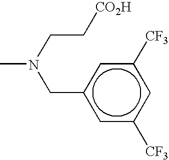 |
| 119. | H | O | 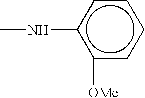 | 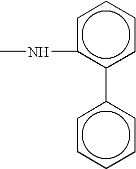 | 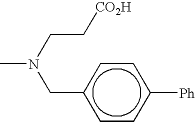 |
| 120. | H | O | 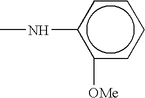 | 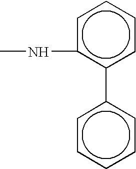 | 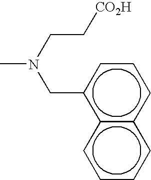 |
| 121. | H | O | 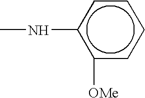 | 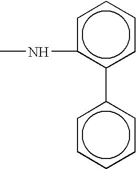 | 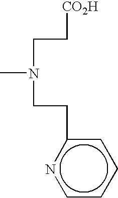 |
| 122. | H | O | 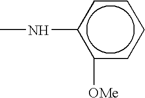 | 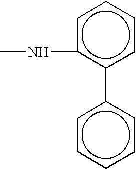 | 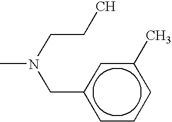 |
| 123. | H | O | 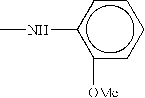 | 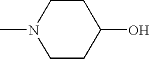 | 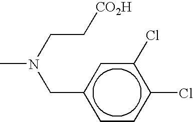 |
| 124. | H | O | 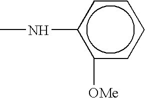 | 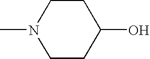 | 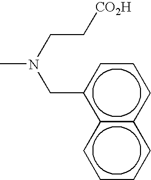 |
| 125. | H | O | 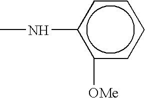 | 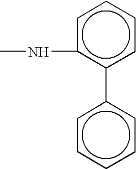 | 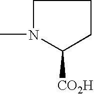 |
| 126. | H | O | 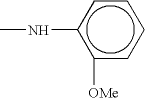 | 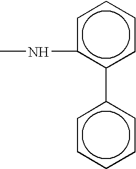 | 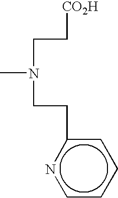 |
| 127. | H | O | 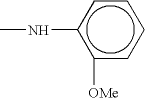 | 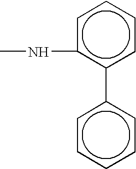 | 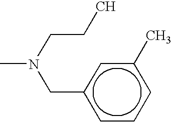 |
| 128. | H | O | 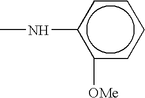 | 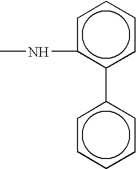 | 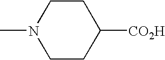 |
| 129. | H | O | 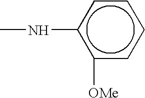 | 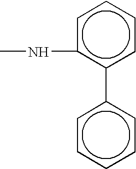 | 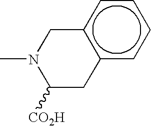 |
| 130. | H | O | 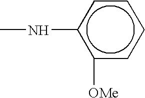 | 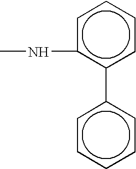 | 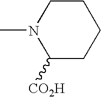 |
| 131.* | 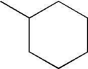 | S | 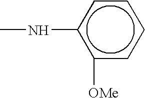 | 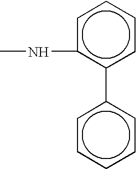 | 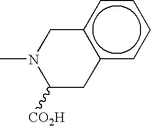 |
| 132.* | H | N(CN) | 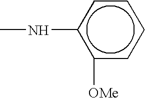 | 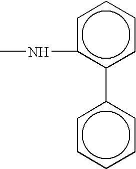 | 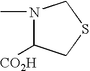 |
| 133.* | H | N(CN) | 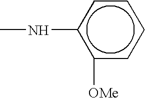 | 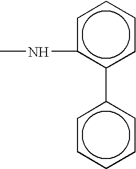 | 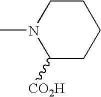 |
| 134.* | H | CH(NO₂) | 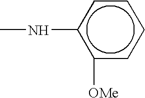 | 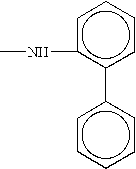 | 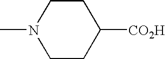 |
| 135.* | CH₃ | O | 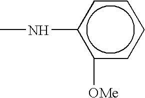 | 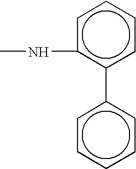 | 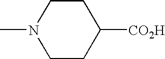 |

*represents hypothetical examples

The present invention also includes within its scope the enantiomers, diastereomers, N-oxides and pharmaceutically acceptable salts of these compound as well as metabolites having the same type of activity.

In the above syntheses, where specific acids, bases, solvents, catalysts, oxidizing agents, reducing agents etc. are mentioned, at is to be understood that the other acids, bases, solvents, catalysts, oxidizing agents, reducing agents etc. may be used. Similarly, the reaction temperature and duration of the reaction may be adjusted as desired.

### EXPERIMENTAL DETAILS

Various solvents, such as acetone, methanol, ether, tetrahydrofuran, hexanes, and dichloromethane, were dried using various drying reagents according to procedures described in the literature. IR spectra were recorded as Nujol mulls or a thin neat film on a Perkin Elmer Paragon instrument. Nuclear Magnetic Resonance (NMR) data (H, C) were recorded on a Varian XL-300 MHz instrument using tetramethylsilane as an internal standard. Chemical Ionization Mass Spectra (CIMS) were obtained using a Finnigan MAT-4510 mass spectrometer equipped with an INCOS data system. Generally, a direct exposure probe and methane as the reagent gas (0.33 mm Hg, 120° C. source temperature) were used.

### Example 1

### Synthesis of 3-[{4-(Biphenyl-4-ylamino)-6-[3-(2-methoxyphenyl)ureido]-[1,3,5]triazin-2-yl}-(4-methylbenzyl)amino]propionic acid (Compound No. 32)

### Step 1: Synthesis of N-Biphenyl-2-yl-6-chloro-[1,3,5]triazine-2,4-diamine

To 2-amino-4,6-dichloro-[1,3,5]triazine (prepared as described in J. Chem. Soc., 1960, 4525) (5 g, 30 mmol) and potassium carbonate (4.2 g, 30 mmol) in acetone (50 ml) at 0° C. were added dropwise to a solution of 2-aminobiphenyl (5.15 g, 30 mmol) in acetone (50 ml). The reaction mixture was then stirred at room temperature for 2 hour and at 40-50° C. for 8 hour. The reaction mixture was poured over ice-water and extracted with ethyl acetate (2×200 ml). The combined organic extracts were washed with brine and dried over anhydrous Na₂SO₄. Evaporation of the solvent followed by purification of the residue over a silica gel column using 30% EtOAc-hexane as eluent furnished the title compound (2.6 g, 29%) as a white solid.

¹HNMR (300 MHz, CDCl₃): δ 8.09 (1H, d, J=8.1 Hz) and 7.1-7.40 (8H, m), [aromatic], 6.98 (1H, bs, NH) and 5.37 (2H, bs, NH₂).

LCMS (m/z): 298.0 (M+1, 55%).

### Step 2: Synthesis of 1-[4-(Biphenyl-2-ylamino)-[1,3,5]triazin-2-yl-3-(2-methoxyphenyl)urea (Compound No. 17)

To the compound obtained from Step 1 above (1 g, 3.3 mmol) in tetrahydrofuran at 0° C. was added sodium hydride (NaH) (158 mg, 50% dispersion in oil, 3.3 mmol) immediately followed by 2-methoxyphenyl isocyanate (0.5 g, 3.3 mmol), The reaction mixture was stirred at 0° C. for 30 min and at room temperature for 12 h. The reaction mixture was then poured into cold water (100 ml) and extracted with ethyl acetate (3×50 ml). The combined organic extracts were washed with water and brine and dried over anhydrous Na₂SO₄. The solvent was then evaporated to obtain a sticky mass that was treated with hexane (100 ml) and dichloromethane (10 ml). The precipitated white solid was filtered and dried under vacuum to obtain the title compound (1.4 g, 95%).

m.p.: 239° C.

¹HNMR (DMSO-d₆, 300 MHz): δ 11.15 (bs), 10.81 (bs) 10.26 (bs) and 10.12 (bs) [NH×3] 6.80-8.20 [13H, m, aromatic] and 3.70 (3H, bs, OCH₃).

LCMS (m/z): 447.1 (M+1, 45%)

### Step 3: Synthesis of 3-[{4-(Biphenyl-2-ylamino)-6-[3-(2-methoxyphenyl) ureido]-[1,3,5]triazin-2-yl}-(4-methylbenzyl)amino]propionic acid (Compound No. 32)

To the lithium salt of the 3-aminopropionic acid derivative (obtained by the hydrolysis of ethyl 3-(4-methylbenzyl)amino propionate (198 mg, 0.89 mmol) with LiOH in H₂O (38 mg, 0.89 mmol) in THF (10 ml)) was added the chlorotriazine (400 mg, 0.89 mmol) as obtained from Step 2. The reaction mixture was refluxed for 6 hours, cooled and poured over ice cold water. It was then extracted with ethyl acetate (2×50 ml) and the combined organic extracts here washed with water and brine and dried with anhydrous sodium sulfate. Evaporation of the solvent followed by purification of the residue over a silica gel column using 50% EtOAc-Hexane as eluent furnished the title compound (280 mg, 52%)

m.p.: 102° C.

¹HNMR (DMSO-d₆, 300 MHz): δ10.91 (1H, s, NH), 9.50 (1H, s, NH), 8.28, (1H, bs, NH), 7.91 (1H, s), 7.57 (d, J=6 Hz) and 7.740 (d, J=6 Hz), [1H], 6.85-7.40 (15H, m) [aromatic], 4.85 (s) & 4.66 (s), [2H, NCH₂Ar], 3.71 (1H, m), 3.52 (s) & 3.49 (s) [3H, OCH₃], 2.47 (m, CH₂CO₂H) and 2.27 (s) & 2.25 (s) [3H, ArCH₃].

LCMS (m/z): 604.2 (M+1, 100%).

The following compounds were prepared similarly:

### 1-{4-(Biphenyl-2-ylamino}-6-[2-cyanoethyl)-(3-methylbenzyl)-amino]-[1,3,5] triazin-2-yl}-3-(2-methoxyphenyl)urea (Compound No. 122)

1-{4-(Biphenyl-2-ylamino}-6-[2-cyanoethyl)-(3-methylbenzyl)-amino]-[1,3,5] triazin-2-yl}-3-(2-methoxyphenyl)urea was prepared following the procedure of Example 1, by using 3-(3-methylbenzyl)amino propiononitrile in place of 3-(4-methylbenzyl)amino propionic acid in Step 3 of Example 1.

m.p.: 114-116° C.

¹HNMR (DMSO-d₆, 300 MHz): δ 10.86 (1H, bs, NH), 9.57 (1H, bs, NH) 8.45 (1H, bs, NH), 6.90-7.90 (17H, m, aromatic), 4.56 (s) & 4.69 (s) [2H, NCH₂Ar], 3.82 (2H, t, NCH₂), 3.54 (3H, s, OCH₃), 2.78 (t) & 2.75 (t, J=9 Hz) [2H, CH₂CN] and 2.25 (s) & 2.19 (s) [3H, Ar CH₃].

LCMS (m/z): 585.4 (M+1, 100%).

### {4-Biphenyl-2-ylamino)-6-[3-(2-methoxyphenyl)ureido]-[1,3,5]triazin-2-yl amino]acetic acid (Compound No. 20)

{4-Biphenyl-2-ylamino)-6-[3-(2-methoxyphenyl)ureido]-[1,3,5]triazin-2-yl amino]acetic acid was prepared as described in Example 1 by using glycine instead of 3-(4-methylbenzyl)aminopropionic acid in Step 3 of Example 1.

m.p.: 210° C.

¹HNMR (DMSO-d₆, 300 MHz): δ 11.82 (s) & 10.99 (s) [1H, NH], 9.45 (s) & 9.43 (s) [1H, NH], 8.32 (1H, bs, NH), 8.15 (d, J=7.2 Hz) & 7.94 (d, J=7.5 Hz), [1H], 7.74 (1H, d, J=7.5 Hz), 7.20-7.50 (9H, m), 7.00 (3H, bs) and 6.89 (bs) & 6.47 (bs) [1H]

[aromatic], 4.02 (3H, s, OCH₃) and 3.72 (s) & 3.64 (s) [N CH₂].

LCMS (m/z): 486.2 (M+1, 35%).

### 2S-{4-(Biphenyl-2-ylamino)-6-[3-(2-methoxyphenyl)ureido]-[1,3,5]triazin-2-yl amino}-3-phenylpropionic acid (Compound No. 21)

2S-{4-(Biphenyl-2-ylamino)-6-[3-(2-methoxyphenyl)ureido]-[1,3,5]triazin-2-yl amino}-3-phenylpropionic acid was prepared as described in Example 1, by using 2-amino-3-phenylpropionic acid (2-phenylalanine) in Step 3, Example 1, instead of 3-(4-methylbenzyl)amino propionic acid.

m.p.: 140° C.

¹HNMR (CDCl₃, 300 MHz): δ 11.84 (1H, bs, NH), 9.93 (1H, bs, NH), 8.75 (1H, bs NH), 8.19 (1H, bs), 7.89 (1H, bs) and 6.72-7.40 (16H, m) [aromatic], 4.80 (1H, bs NCH), and 3.07-3.27 (5H, m, OCH₃ and CH₂Ar).

IR (νₘₐₓ, KBr): 1685.3, 1479.4, 1341.9, 816.8, 747.1 cm⁻¹.

LCMS (M/Z): 576.1 (M+1, 85%).

### 3-{4-Biphenyl-2-ylamino)-6-[3-(2-methoxyphenyl)ureido]-[1,3,5]triazin-2-yl amino}-3-phenylpropionic acid (Compound No. 22)

3-{4-Biphenyl-2-ylamino)-6-[3-(2-methoxyphenyl)ureido]-[1,3,5]triazin-2-yl amino}-3-phenylpropionic acid was prepared by using 3-amino-3-phenylpropionic acid in Step 3, Example 1, instead of 3-(4-methylbenzyl)aminopropionic acid.

m.p.: 151° C.

¹HNMR (DMSO-D₆, 300 MHz): δ 11.33 (bs) & 10.95 (bs) [1H, NH], 9.45 (bs) & 9.41 (bs) [1H, NH], 8.06 (d, J=9.3 Hz), 7.88 (d) [2H, 1NH], 7.72 (d) & 7.69 (d, J=8.4 Hz) [1H] and 6.90-7.35 (18H) [aromatic], 5.43 (1H, bs) & 5.15 (1H, bs) [NCH], 3.64 (s) & 3.59 (s) [3H, OCH₃] and 2.50-2.88 (2H, m, CH₂CO).

IR (νₘₐₓ, KBr): 1692.1, 1600.6, 1271.0, 748.3 cm⁻¹.

LCMS (m/z) 576.2 (M+1, 100%).

### 3-{3-Benzo[1,3]dioxo-5-yl-{4-(biphenyl-2-ylamino)-6-[3-(2-methoxyphenyl) ureido]-[1,3,5]triazin-2-ylamino}propionic acid (Compound No. 23)

3-{3-Benzo[1,3]dioxo-5-yl-{4-(biphenyl-2-ylamino)-6-[3-(2-methoxyphenyl) ureido]-[1,3,5]triazin-2-ylamino}propionic acid was prepared by using 3-amino-3-benzo[1,3]dioxo-5-ylpropionic acid in place of 3-(4-methylbenzyl)aminopropionic acid in Step 3, of Example 1.

¹HNMR (DMSO-d₆, 300 MHz): δ 11.36 (bs) & 10.97 (bs) [1H, NH], 9.49 (s) & 9.40 (s) [1H, NH], 7.6-7.97 (4H, m), 7.25-7.60 (8H, m), and 6.68-7.0 (6H, m) [aromatic and 2 NH], 5.95 (s) & 5.92 (s) [OCH₂O], 5.35 (m) & 5.17 (bs) [1H, NCH], 3.61 (s) & 3.60 (s) [3H, OCH₃] and 2.50-2.86 (2H, m, CH₂CO).

LCMS (m/z): 600.0 (M+1, 100%).

### 3-{3-Benzo[1,3]dioxo-5-yl-{4-(biphenyl-2-ylamino)-6-[3-(2-methoxyphenyl) ureido]-[1,3,5]triazin-2-ylamino}propionic acid (Compound No. 24)

3-{3-Benzo[1,3]dioxo-5-yl-{4-(biphenyl-2-ylamino)-6-[3-(2-methoxyphenyl) ureido]-[1,3,5]triazin-2-ylamino}propionic acid was synthesized by using 2S-amino-4-methylpentanoic acid (L-Leucine) instead of 3-(4-methylbenzyl)aminopropionic acid in Step 3, Example 1.

m.p.: 123° C.

¹HNMR (DMSO-d₆, 300 MHz): δ 12.64 (1H, bs, CO₂H), 11.39 (bs) & 10.75 (bs) [1H, NH], 9.44 (1H, s, NH), 8.10 (1H, bs, NH), 6.87-8.06 (1H, m, aromatic), 4.52 (t) & 4.39 (bs) [1H, NCHCO], 3.64 (s) & 3.56 (s) [3H, OCH₃], 2.18 (t) & 1.89 (m) [1H CH], 1.23-1.66 (2H, m, CH₂) and 0.88 (d, J=6 Hz), 0.84 (d, J=6 Hz) & 0.82 (d, J=5.7 Hz) [6H, CH₃×2].

IR (νₘₐₓ, KBr): δ 1718.0, 1694.3, 1599.3, 747.3

LCMS (m/z): 548.0 (M+1, 80%).

### 2-[4-(Biphenyl-2-ylamino)-6-[3-(2-methoxyphenyl)ureido]-[1,3,5]triazin-2-ylamino}benzoic acid (Compound No. 25)

2-[4-(Biphenyl-2-ylamino)-6-[3-(2-methoxyphenyl)ureido]-[1,3,5]triazin-2-ylamino}benzoic acid was prepared by using 2-aminobenzoic acid in place of 3-(4-methyl benzyl)aminopropionic acid in Step 3, Example 1.

m.p.: 228° C.

¹HNMR (DMSO-d₆, 300 MHz): δ 11.0 (2H, bs, NH and CO₂H), 9.81 (1H, s, NH), 9.04 (1H, bs, NH), 8.63 (1H, bs, NH), 7.97 (2H, d, J=7.2 Hz), 7.55 (1H, d, J=7.05 Hz), 7.31 (9H, bs) and 6.87-7.06 (4H, m) [aromatic] and 3.61 (3H, s, OCH₃).

IR (νₘₐₓ, KBr): 1680.4, 1545.6, 1254.7, 746.6 cm⁻¹.

LCMS (m/z): 548.2 (M+1, 20%).

### 3-[{(Benzo[1,3]dioxo-5-yl)methyl-{4-biphenyl-2-ylamino}-6-[3-(2-methoxy phenyl)ureido]-[1,3,5]triazin-2-yl]}amino]propanoic acid (Compound No 26)

3-[{(Benzo[1,3]dioxo-5-yl)methyl-{4-biphenyl-2-ylamino}-6-[3-(2-methoxy phenyl)ureido]-[1,3,5]triazin-2-yl]}amino]propanoic acid was prepared by using 3-benzo[1,3]dioxo-5-yl)methylamino propanoic acid in place of 3-(4-methylbenzyl)aminopropanoic acid in Step 3, Example 1.

m.p.: 97° C.

¹HNMR (DMSO-d₆, 300 MHz): δ 10.88 (1H, NH), 9.53 (s) & 9.46 (s) [1H, NH], 7.90 (1H, bs), 7.41 (bs) & 7.62 (bs) [1NH], 7.00-7.50 (9H, m), 6.6-7.00 (6H, m) [aromatic], 5.95 (s) & 5.94 (s) [2H, OCH₂O], 4.75 (s) & 4.57 (s) [2H, NCH₂Ar], 3.67 (1H, t, J=6 Hz), 3.51 (s) & 3.46 (s) [3H, OCH₃] and 2.42 (2H, m, CH₂CO₂H).

IR (νₘₐₓ, KBr): 1709.1, 1533.8, 1036.8, 746.9 cm⁻¹.

LCMS (m/z): 634.3 (M+1, 100%).

### 3-[{4-Biphenyl-2-yl-amino}-6-[3-(2-methoxyphenyl)ureido][1,3,5]triazin-2-yl}-(3,4-dimethoxybenzyl)amino]propionic acid (Compound No. 27)

3-[{4-Biphenyl-2-yl-amino}-6-[3-(2-methoxyphenyl)ureido][1,3,5]triazin-2-yl}-(3,4-dimethoxybenzyl)amino]propionic acid was prepared by using 3-(3,4-dimethoxybenzyl)aminopropionic acid instead of 3-(4-ethylbenzyl)aminopropionic acid in Step 3, Example 1.

m.p.: 97° C.

¹HNMR (DMSO-d₆, 300 MHz): δ 12.20 (1H, bs, CO₂H), 10.94 (1H, bs, NH), 9.53 (s) & 9.50 (s) [1H, NH], 8.22 (1H, bs, NH), 7.93 (1H, s), 7.42 (1H, d, J=9 Hz) & 7.34 (1H, d, J=9 Hz) 7.20-7.45 (8H, m) & 6.73-7.00 (6H, m) [aromatic], 4.80 (s) & 4.64 (s) [2H, NCH₂Ar], 3.72 (s), 3.70 (s), 3.62 (s) 3.57 (s), 3.51 (s) and 3.40 (s) [11H, OCH₃×3 and NCH₂] and 2.47 (2H, m, CH₂CO₂H).

IR (νₘₐₓ, KBr): 1706.1, 1606.3, 1258.8, 749.0 cm⁻¹.

LCMS (m/z) 650.2 (M+1, 100%).

### 3-[{4-Biphenyl-2-ylamino)-6-[3-(2-methoxyphenyl)ureido]-[1,3,5]triazin-2-yl}-(4-methoxybenzyl)amino]propionic acid (Compound No. 28)

3-[{4-Biphenyl-2-ylamino)-6-[3-(2-methoxyphenyl)ureido]-[1,3,5]triazin-2-yl}-(4-methoxybenzyl)amino]propionic acid was synthesized by using 3-(4-methoxybenzyl)amino propionic acid in place of 3-(4-methylbenzyl)aminopropionic acid in Step 3, Example 1.

m.p.: 95° C.

¹HNMR (DMSO-d₆, 300 MHz): δ 10.92 (1H, bs, NH), 9.52 (s) & 9.49 (s) [1H, NH], 8.2 (1H, bs, NH), 7.94 (1H, t, J=6 Hz), 6.84-7.6 (16H, m) [aromatic], 4.82 (s) & 4.63 (s) [2H, NCH₂Ar] 3.72 (s), 3.71 (s), 3.53 (s) & 3.48 (s) [8H, OCH₃×2 and NCH₂] and 2.46 (2H, m, CH₂CO₂H).

IR (νₘₐₓ, KBr): 1708.3, 1609.9, 1509.0, 747.9 cm⁻¹.

LCMS (m/z): 620.3 (M+1, 100%).

LCMS (m/z): 634.3 (M+1, 100%).

### 3-[{4-Biphenyl-2-yl-amino}-6-[3-(2-methoxyphenyl)ureido][1,3,5]triazin-2-yl}-(3,4-dimethoxybenzyl)amino]propionic acid (Compound No. 27)

3-[{4-Biphenyl-2-yl-amino}-6-[3-(2-methoxyphenyl)ureido][1,3,5]triazin-2-yl}-(3,4-dimethoxybenzyl)amino]propionic acid was prepared by using 3-(3,4-dimethoxybenzyl)aminopropionic acid instead of 3-(4-ethylbenzyl)aminopropionic acid in Step 3, Example 1.

m.p.: 97° C.

¹HNMR (DMSO-d₆, 300 MHz): δ 12.20 (1H, bs, CO₂H), 10.94 (1H, bs, NH), 9.53 (s) & 9.50 (s) [1H, NH], 8.22 (1H, bs, NH), 7.93 (1H, s), 7.42 (1H, d, J=9 Hz) & 7.34 (1H, d, J=9 Hz) 7.20-7.45 (8H, m) & 6.73-7.00 (6H, m) [aromatic], 4.80 (s) & 4.64 (s) [2H, NCH₂Ar], 3.72 (s), 3.70 (s), 3.62 (s) 3.57 (s), 3.51 (s) and 3.40 (s) [11H, OCH₃×3 and NCH₂] and 2.47 (2H, m, CH₂CO₂H).

IR (νₘₐₓ, KBr): 1706.1, 1606.3, 1258.8, 749.0 cm⁻¹.

LCMS (m/z) 650.2 (M+1, 100%).

### 3-[{4-Biphenyl-2-ylamino)-6-[3-(2-methoxyphenyl)ureido]-[1,3,5]triazin-2-yl}-(4-methoxybenzyl)amino]propionic acid (Compound No. 28)

3-[{4-Biphenyl-2-ylamino)-6-[3-(2-methoxyphenyl)ureido]-[1,3,5]triazin-2-yl}-(4-methoxybenzyl)amino]propionic acid was synthesized by using 3-(4-methoxybenzyl)amino propionic acid in place of 3-(4-methylbenzyl)aminopropionic acid in Step 3, Example 1.

m.p.: 95° C.

¹HNMR (DMSO-d₆, 300 MHz): δ 10.92 (1H, bs, NH), 9.52 (s) & 9.49 (s) [1H, NH], 8.2 (1H, bs, NH), 7.94 (1H, t, J=6 Hz), 6.84-7.6 (16H, m) [aromatic], 4.82 (s) & 4.63 (s) [2H, NCH₂Ar] 3.72 (s), 3.71 (s), 3.53 (s) & 3.48 (s) [8H, OCH₃×2 and NCH₂] and 2.46 (2H, m, CH₂CO₂H).

IR (νₘₐₓ, KBr): 1708.3, 1609.9, 1509.0, 747.9 cm⁻¹.

LCMS (m/z): 620.3 (M+1, 100%).

### 3-[{4-Biphenyl-2-ylamino)-6-[3-(2-methoxyphenyl)ureido]-[1,3,5]triazin-2-yl}-(3-methoxybenzyl)-amino]propionic acid (Compound No. 29)

3-[{4-Biphenyl-2-ylamino)-6-[3-(2-methoxyphenyl)ureido]-[1,3,5]triazin-2-yl}-(3-methoxybenzyl)-amino]propionic acid was synthesized by using 3-(3-methoxybenzyl)aminopropionic acid in place of 3-(4-ethylbenzyl)aminopropionic acid in Step 3, Example 1.

m.p.: 93° C.

¹HNMR (DMSO-d₆, 300 MHz): δ 12.05 (1H, bs, CO₂H), 10.91 (1H, s, NH), 9.52 (1H, s, NH), 8.25 (1H, bs, NH), 7.90 (1H, s), 7.20-7.75 (10H, m) & 6.75-7.20 (6H, m) [aromatic], 4.86 (s) & 4.70 (s) [NCH₂Ar], 3.76 (s), 3.75 (s) & 3.65 (s) [8H, OCH₃×2 and NCH₂] and 2.50

(DMSO+2H of CH₂CO₂H).

IR (νₘₐₓ, KBr): 1709.2, 1535.2, 1254.6, 746.8 cm⁻¹.

LCMS (m/z): 620.1 (M+1, 100%).

### 3-[{4-Biphenyl-2-ylamino)-6-[3-(2-methoxyphenyl)-ureido]-[1,3,5]triazin-2-yl]-(2-methoxybenzyl)amino]propionic acid (Compound No. 30)

3-[{4-Biphenyl-2-ylamino)-6-[3-(2-methoxyphenyl)-ureido]-[1,3,5]triazin-2-yl]-(2-methoxybenzyl)amino]propionic acid was prepared by using 3-(2-methoxybenzyl)aminopropionic acid in place of 3-(4-ethylbenzyl)aminopropionic acid in Step 3, Example 1.

m.p.: 125-132° C.

¹HNMR (TFA-d, 300 MHz): δ 7.80-8.60 (18H, m, aromatic and 1 NH), 5.87 (s) & 5.77 (s) [2H, NCH₂Ar], 4.75-5.05 (2H, m, NCH₂), 4.83 (s), 4.81 (s) & 4.79 (s) [6H, OCH₃×2] and 3.58 (bs) & 3.03 (bs) [2H, CH₂CO₂H].

IR (νₘₐₓ, KBr): 1708.5, 1579.1, 1505.9, 742.3 cm⁻¹.

LCMS (m/z): 620.3 (M+1, 100%).

### 3-[{4-Biphenyl-2-ylamino)-6-[3-(2-methoxyphenyl)ureido]-[1,3,5]triazin-2-yl}-(3-methylbenzyl)amino]propionic acid (Compound No. 31)

3-[{4-Biphenyl-2-ylamino)-6-[3-(2-methoxyphenyl)ureido]-[1,3,5]triazin-2-yl}-(3-methylbenzyl)amino]propionic acid was prepared by using 3-(3-methylbenzyl)aminopropionic acid instead of 3-(4-methylbenzyl)aminopropionic acid in Step 3, Example 1.

m.p.: 205-212° C.

¹HNMR (TFA-d, 300 MHz) δ 7.80-8.40 (18H, m, aromatic & 1 NH), 5.82 (s) & 5.72 (s) [2H, NCH₂Ar], 4.75-5.25 (2H, m, NCH₂], 4.85 (s) & 4.83 (s) [3H, OCH₃], 3.74 (bs) & 3.56 (bs) [2H, CH₂CO₂H] and 3.26 (s) & 3.23 (s) [3H, ArCH₃].

LCMS (m/z): 604.2 (M+1, 100%).

### 3-[{4-Biphenyl-2-ylamino)-6-[3-(2-methoxyphenyl)ureido]-[1,3,5]triazin-2-yl}-(2-fluorobenzyl)amino]propionic acid (Compound No. 33)

3-[{4-Biphenyl-2-ylamino)-6-[3-(2-methoxyphenyl)ureido]-[1,3,5]triazin-2-yl}-(2-fluorobenzyl)amino]propionic acid was prepared by using 3-(2-fluorobenzyl)aminopropionic acid in place of 3-(4-methylbenzyl)aminopropionic acid in Step 3, Example 1.

m.p.: 208-215° C.

¹HNMR (TFA-d, 300 MHz): δ 7.83-8.45 (20H, m, aromatic and 3 NH), 5.94 (s) & 5.79 (s) [2H, NCH₂Ar], 5.02 (1H, bs) & 4.87 (4H, bs) [NCH₂ and OCH₃], 3.79 (bs) & 3.61 (bs) [2H, CH₂CO₂H].

LCMS (M/Z): 608.2 (m+1, 50%).

### 3-[{4-(Biphenyl-2-ylamino)-6-[3-(2-methoxyphenyl)ureido]-[1,3,5]triazin-2-yl}-(4-fluorobenzyl)amino]propionic acid (Compound No. 34)

3-[{4-(Biphenyl-2-ylamino)-6-[3-(2-methoxyphenyl)ureido]-[1,3,5]triazin-2-yl}-(4-fluorobenzyl)amino]propionic acid was prepared by using 3-(4-fluorobenzyl)aminopropionic acid instead of 3-(4-methylbenzyl)aminopropionic acid in Step 3, Example 1.

m.p.: 108° C.

¹HNMR (DMSO-d₆, 300 MHz): δ 10.90 (1H, bs, NH), 9.53 (s) & 9.51 (s) [1H, NH], 6.89-8.30 (19H, m, 17H aromatic and 2 NH), 4.86 (s) & 4.67 (s) [2H, NCH₂Ar], 3.83 (2H, m, NCH₂) and 3.54 (s) & 3.49 (s) [3H, OCH₃].

IR (νₘₐₓ, KBr): 1710.0, 1608.1, 1537.7, 746.7 cm⁻¹. dichlorobenzyl)aminopropionic acid instead of 3-(4-methylbenzyl)aminopropionic acid in Step 3, Example 1.

m.p.: 185-192° C.

¹HNMR (TFA-d, 300 MHz): δ 7.75-8.40 (19H, m, 16H aromatic and 3 NH), 5.96 (s) & 5.81 (s) [2H, NCH₂Ar], 4.7-5.1 (2H, m, NCH₂), 4.86 (3H, bs, OCH₃) and 3.78 (bs) & 3.61 (bs) [2H, CH₂CO₂H].

IR (νₘₐₓ, KBr): 1719.6, 1660.6, 1262.9, 1020.8, 744.6 cm⁻¹.

LCMS (m/z): 658.2 (M+1, 100%).

### 3-[{4-(Biphenyl-2-ylamino]-6-[3-(2-methoxyphenyl)ureido]-[1,3,5]triazin-2-yl}amino]propionic acid (Compound No. 38)

3-[{4-(Biphenyl-2-ylamino]-6-[3-(2-methoxyphenyl)ureido]-[1,3,5]triazin-2-yl}amino]propionic acid was prepared by using 3-aminopropionic acid (β-alanine) instead of 3-(4-methyl benzyl)aminopropionic acid in Step 3, Example 1.

m.p.: 196-205° C.

¹HNMR (TFA-d, 300 MHz): δ 8.10-8.85 (13H, m, aromatic), 5.01 (5H, bs, OCH₃ and NCH₂) and 3.91 (2H, bs, CH₂CO₂H).

IR (νₘₐₓ, KBr): 1690.1, 1600.8, 1549.5, 1251.8, 747.4 cm⁻¹.

LCMS (m/z): 500.2 (M+1, 100%).

### [{4-(Biphenyl-2-ylamino)-6-[3-(2-methoxyphenyl)ureido]-[1,3,5]triazin-2-yl}-(4-methylbenzyl)amino]acetic acid (Compound No. 39)

[{4-(Biphenyl-2-ylamino)-6-[3-(2-methoxyphenyl)ureido]-[1,3,5]triazin-2-yl}-(4-methylbenzyl)amino]acetic acid was prepared by using 3-(4-methylbenzyl)amino acetic acid instead of 3-(4-methylbenzyl)amino propionic acid in Step 3, Example 1.

m.p.: 132-145° C.

¹HNMR (DMSO-d₆, 300 MHz): δ 10.90 (s) & 10.84 (s) [1H, NH], 9.58 (s) & 9.49 (s) [1H, NH], 8.32 (1H, bs, NH), 7.90 (1H, t), 7.66 (d) & 7.40 (d) [1H, J=3 Hz], 6.85-7.35 [15H, m] [aromatic], 4.83 (s), 4.62 (s), 4.27 (s) & 4.01 (s) [4H, NCH₂×2], 3.59 (3H, s, OCH₃) and 2.26 (s) & 2.25 (s) [3H, Ar CH₃].

IR (νₘₐₓ, KBr): 1696.0, 1598.7, 1507.1, 1246.3, 748.0 cm⁻¹.

LCMS (m/z): 590.0 (M+1, 100%).

### [{4-(Biphenyl-2-ylamino]-6-[3-(2-methoxyphenyl)ureido]-[1,3,5]triazin-2-yl}-(4-methoxybenzyl)amino]acetic acid (Compound No. 40)

[{4-(Biphenyl-2-ylamino]-6-[3-(2-methoxyphenyl)ureido]-[1,3,5]triazin-2-yl}-(4-methoxybenzyl)amino]acetic acid was prepared by using 2-(4-methoxybenzyl)aminoacetic acid instead of 3-(4-methylbenzyl)aminopropionic acid in Step 3, Example 1.

m.p.: 147-149° C.

¹HNMR (DMSO-d₆, 300 MHz): δ 12.5 (1H, bs, CO₂H), 10.92 (s) & 10.85 (s) [1H, NH], 9.61 (s) & 9.50 (s) [1H, NH], 8.3 (1H, bs, NH), 7.92 (1H, t), 7.70 (d) & 7.59 (d) [1H] and 6.80-7.50 (15H, m) [aromatic], 4.80 (s) & 4.58 (s), 4.27 (s) & 4.01 (s) [4H, NCH₂×2] and 3.74 (s), 3.71 (s), 3.59 (s) & 3.49 (s) [6H, OCH₃×2].

IR (νₘₐₓ, KBr): 1692.9, 1597.6, 1508.6, 1246.6, 748.2 cm⁻¹.

LCMS (m/z): 606.3 (M+1, 20%).

### [{4-(Biphenyl-2-ylamino)-6-[3-(2-methoxyphenyl)ureido]-[1,3,5]triazin-2-yl}-(3,4-dimethoxybenzyl)amino]acetic acid (Compound No. 41)

[{4-(Biphenyl-2-ylamino)-6-[3-(2-methoxyphenyl)ureido]-[1,3,5]triazin-2-yl}-(3,4-dimethoxybenzyl)amino]acetic acid was prepared by using 2-(3,4-dimethoxybenzyl)aminoacetic acid in place of 3-(4-methylbenzyl)aminopropionic acid in Step 3, Example 1.

m.p.: 115-120° C.

¹HNMR (DMSO-d₆, 300 MHz): δ 12.5 (1H, bs, CO₂H), 10.92 (s) &10.85 (s) [1H, NH], 9.61 (s) & 9.50 (s) [1H, NH], 8.3 (1H, bs, NH), 7.92 (1H, t), 7.70 (d), 7.59 (d) [1H] and 6.80-7.50 (15H, m) [aromatic], 4.80 (s), 4.58 (s), 4.27 (s) & 4.01 (s) [4H, NCH₂×2] and 3.74 (s), 3.71 (s), 3.59 (s) & 3.49 (s) [6H, OCH₃×2].

IR (νₘₐₓ, KBr): 1692.9, 1597.6, 1508.6, 1246.6, 748.2 cm⁻¹.

LCMS (m/z): 606.3 (M+1, 20%).

### [{4-(Biphenyl-2-ylamino)-6-[3-(2-methoxyphenyl)ureido]-[1,3,5]triazin-2-yl}-[4-fluorobenzyl]amino]acetic acid (Compound No. 42)

[{4-(Biphenyl-2-ylamino)-6-[3-(2-methoxyphenyl)ureido]-[1,3,5]triazin-2-yl}-[4-fluorobenzyl]amino]acetic acid was prepared by using 3-(4-fluorobenzyl)aminoacetic acid instead of 3-(4-methylbenzyl)aminopropionic acid in Step 3, Example 1.

m.p.: 120-125° C.

¹HNMR (DMSO-d₆, 300 MHz): δ 10.87 (s) & 10.81 (s) [1H, NH], 9.60 (s) & 9.49 (s) [1H, NH], 8.3 [1H, bs, NH], 7.89 (1H, t), 7.64 (d) & 7.53 (d) [1H] and 6.80-7.40 (15H, m) [aromatic], 4.83 (s), 4.60 (s), 4.31 (s) & 4.04 (s) [4H, NCH₂×2] and 3.59 (s) & 3.44 (s) [3H, OCH₃].

IR (νₘₐₓ, KBr): 1695.9, 1599.2, 1535.2, 1221.3, 747.7 cm⁻¹.

LCMS (m/z): 594.1 (M+1, 100%).

### 3-[{4-(Biphenyl-2-ylamino]-6-[3-(2-methoxyphenyl)ureido]-[1,3,5]triazin-2-yl}-(4-trifluoromethyl benzyl)amino]propionic acid (Compound No. 73)

3-[{4-(Biphenyl-2-ylamino]-6-[3-(2-methoxyphenyl)ureido]-[1,3,5]triazin-2-yl}-(4-trifluoromethyl benzyl)amino]propionic acid was prepared by using 3-(4-trifluoromethylbenzyl)aminopropionic acid instead of 3-(4-methylbenzyl)aminopropionic acid in Step 3, Example 1.

m.p.: 125-128° C.

¹HNMR (TFA-d, 300 MHz): δ 8.40-9.00 (17H, m, aromatic), 6.33 (s) & 6.18 (s) [2H, NCH₂Ar], 5.40 (m) & 5.24 (m) [2H, NCH₂], 5.30 (s) & 5.18 (s) [3H, OCH₃] and 4.22 (t) & 4.03 (t) [2H, CH₂CO₂H].

IR (νₘₐₓ, KBr): 1707.9, 1533.2, 1325.5, 1123.5, 748.0 cm⁻¹.

LCMS (m/z): 658.3 (M+1, 100%).

### 3-[{4-(Biphenyl-2-ylamino)-6-[3-(2-methoxyphenyl)ureido]-[1,3,5]triazin-2-yl}-(thiophen-2-ylmethyl)amino]propionic acid (Compound No. 74)

3-[{4-(Biphenyl-2-ylamino)-6-[3-(2-methoxyphenyl)ureido]-[1,3,5]triazin-2-yl}-(thiophen-2-ylmethyl)amino]propionic acid was prepared by using 3-(thiophen-2-ylmethyl)aminopropionic acid instead of 3-(4-methylbenzyl)aminopropionic acid in Step 3, Example 1.

m.p.: 115-120° C.

¹HNMR (TFA-d, 300 MHz): δ 8.30-9.04 (19H, m, aromatic and 3NH), 6.43 (s) & 6.36 (s) [2H, NCH₂Ar], 5.44 (m), 5.37 (m), 5.34 (s) & 5.30 (s) [5H, NCH₂ and OCH₃] and 4.19 (bs) & 4.05 (bs) [2H, CH₂CO₂H].

IR (νₘₐₓ, KBr): 1707.2, 1599.0, 1531.6, 1252.1, 748.1 cm⁻¹.

LCMS (m/z): 596.2 (M+1, 100%).

### 3-[{4-(Biphenyl-2-ylamino)-6-[3-(2-methoxyphenyl)ureido]-[1,3,5]triazin-2-yl}-(3,4-difluorobenzyl)amino]propionic acid (Compound No. 75)

3-[{4-(Biphenyl-2-ylamino)-6-[3-(2-methoxyphenyl)ureido]-[1,3,5]triazin-2-yl}-(3,4-difluorobenzyl)amino]propionic acid was prepared by using 3-(3,4-difluorobenzyl)aminopropionic acid instead of 3-(4-methylbenzyl)aminopropionic acid in Step 3, Example 1.

m.p.: 115-120° C.

¹HNMR (TFA-d, 300 MHz): δ 8.40-9.00 (19H, m, aromatic and 3NH), 6.27 (s) & 6.12 (s) [2H, NCH₂Ar], 5.45 (m), 5.34 (s) & 5.20 (m) [5H, NCH₂ and OCH₃] and 4.23 (t) & 4.02 (t) [2H, CH₂CO₂H].

IR (νₘₐₓ, KBr): 1707.7, 1598.7, 1532.8, 1279.5, 748.2 cm⁻¹.

LCMS (m/z): 626.2 (M+1, 100%).

### 3-[{4-(Biphenyl-2-ylamino)-6-[3-(2-methoxyphenyl)ureido]-[1,3,5]triazin-2-yl}-(2-((4-hydroxyphenyl)ethyl)amino]propionic acid (Compound No. 76)

3-[{4-(Biphenyl-2-ylamino)-6-[3-(2-methoxyphenyl)ureido]-[1,3,5]triazin-2-yl}-(2-((4-hydroxyphenyl)ethyl)amino]propionic acid was prepared by reaction 3-(2-(4-hydroxyphenyl)ethyl]aminopropionic acid instead of 3-(4-methylbenzyl)aminopropionic acid in Step 3, Example 1.

m.p.: 109-115° C.

¹HNMR (TFA-d, 300 MHz): δ 8.25-9.04 (17H, m, aromatic), 5.55 (s), 5.40 (bs), 5.26 (bs) & 4.70 (s) [7H, NCH₂×2 and OCH₃], 4.56 (bs), 4.30 (m), 4.14 (m) & 3.90 (m) [4H, CH₂ Ar and CH₂CO₂H].

IR (νₘₐₓ, KBr): 1695.7, 1602.2, 1533.9, 1255.3, 749.5 cm⁻¹.

LCMS (m/z): 620.3 (M+1, 100%).

### 3-[{4-(Biphenyl-2-ylamino)-6-[3-(2-methoxyphenyl)ureido]-[1,3,5]triazin-2-yl}-[2-(1H-indol-3yl)ethyl]amino]propionic acid (Compound No. 77)

3-[{4-(Biphenyl-2-ylamino)-6-[3-(2-methoxyphenyl)ureido]-[1,3,5]triazin-2-yl}-[2-(1H-indol-3yl)ethyl]amino]propionic acid was prepared by using 3-[2-(1H-indol-3-yl)ethyl]aminopropionic acid instead of 3-(4-methylbenzyl)aminopropionic acid in Step 3, Example 1.

m.p.: 115-120° C.

¹HNMR (TFA-d, 300 MHz): δ 8.35-8.85 (18H, m, aromatic), 5.41 (s), 5.31 (bs), 5.27 (bs), 4.99 (s), [7H, NCH₂×2 and OCH₃], 4.44 (bs), 4.24 (bs), 4.15 (bs) & 4.05 bs) [4H, CH₂ Ar and CH₂CO₂H]

IR (νₘₐₓ, KBr): 1699.3, 1530.4, 1252.6, 745.4 cm⁻¹.

LCMS (m/z): 643.3 (M+1, 100%).

### 4-[{4-(Biphenyl-2-ylamino)-6-[-(2-methoxyphenyl)ureido]-[1,3,5]triazin-2-yl}amino]phenylacetic acid (Compound No. 78)

4-[{4-(Biphenyl-2-ylamino)-6-[-(2-methoxyphenyl)ureido]-[1,3,5]triazin-2-yl}amino]phenylacetic acid was prepared by using 4-aminophenylacetic acid instead of 3-(4-methyl benzyl)aminopropionic acid in Step 3, Example 1.

m.p.: >300° C.

¹HNMR (TFA-d, 300 MHz): δ 8.24-8.90 (17H, m, aromatic) and 5.30 (s), 5.15 (s) & 5.08 (bs) [5H, OCH₃ and CH₂CO₂H].

LCMS (m/z): 562.0 (M+1, 100%).

### 3-[{4-(Biphenyl-4-ylamino)-6-[3-(2-methoxyphenyl)ureido]-[1,3,5]triazin-2-yl}-(3-methylbenzyl)amino]propionic acid (Compound No. 82)

3-[{4-(Biphenyl-4-ylamino)-6-[3-(2-methoxyphenyl)ureido]-[1,3,5]triazin-2-yl}-(3-methylbenzyl)amino]propionic acid was prepared by using 4-aminobiphenyl instead of 2-aminobiphenyl as in Step 1, Example 1 and 3-(3-methylbenzyl)aminopropionic acid instead of 3-(4-methylbenzyl)aminopropionic acid in Step 3, Example 1.

¹HNMR (DMSO-d₆, 300 MHz): δ 11.3 (bs) & 10.89 (bs) [1H, NH], 9.70 (bs) & 9.58 (bs), [1H, NH), 6.90-7.94 (17H, m, aromatic), 4.95 (bs) & 4.90 (m) [2H, NCH₂Ar], 2.66 (m, CH₂CO₂H) and 2.29 (s) & 2.19 (s), [3H, ArCH₃].

IR (νₘₐₓ, KBr): 1693.1, 1588.8, 1422.0, 1238.4, 761.7 cm⁻¹.

LCMS (m/z): 604.2 (M+1, 45%).

### 3-[{4-(Biphenyl-4-ylamino)-6-[3-(2-methoxyphenyl)ureido]-[1,3,5]triazin-2-yl}-(3,4-dichlorobenzyl)amino]propionic acid (Compound No. 93)

3-[{4-(Biphenyl-4-ylamino)-6-[3-(2-methoxyphenyl)ureido]-[1,3,5]triazin-2-yl}-(3,4-dichlorobenzyl)amino]propionic acid was prepared by using 4-aminobiphenyl in place of 2-aminobiphenyl as in Step 1, Example 1 and 3-(3,4-dichlorobenzyl)aminopropionic acid instead of 3-(4-methylbenzyl)aminopropionic acid in Step 3, Example 1.

m.p.: 215-220° C.

¹HNMR (DMSO-d₆, 300 MHz): δ 10.86 (1H, bs, NH), 9.76 (s) & 9.63 (s) [1H, NH], 7.65-8.00 (2H, m), 7.30-7.60 (12H, m) and 6.9-7.05 (2H, m) [aromatic], 4.95 (s) & 4.90 (s) [2H, NCH₂ Ar] 3.70-3.90 (5H, m, OCH₃ and NH₂) and 2.67 (1H, t, J=6 Hz) [CH₂CO₂H].

IR (νₘₐₓ, KBr): 1691.4, 1538.7, 1422.1, 761.7 cm⁻¹.

LCMS (m/z): 658.2 (M+1, 100%).

### 3-[{4-(Biphenyl-2-ylamino)-6-[3-(2-hydroxyphenyl)ureido]-[1,3,5]triazin-2-yl}-(3-methylbenzyl)amino]propionic acid (Compound No. 112)

To Compound No. 31 (200 mg, 0.33 mmol) in dry dichloromethane (DCM) (10 ml) at −80° C. was added boron tribromide solution in dichloromethane (1.65 ml of 1M solution, 1.65 mmol) and stirred for 2 hours. It was warmed to room temperature, stirred overnight and poured into water (50 ml). The aqueous layer was extracted with dichloromethane (2×25 ml) and the combined organic extracts were washed with brine and dried (anhydrous sodium sulfate). Evaporation of the solvent followed by purification of the residue over a silica gel column using 1-5% MeOH-DCM afforded the title compound (95 mg, 49%) as a white solid.

m.p.: 122-124° C.

¹HNMR (TFA-d, 300 MHz): δ 7.5-8.10 (17H, m, aromatic), 5.58 (s) & 5.47 (s) [2H, NCH₂ Ar], 4.5-4.75 (2H, m, NCH₂), 3.45 (bs) & 3.31 (bs) [2H, CH₂CO₂H].

IR (νₘₐₓ, KBr): 1687.5, 1533.1, 1284.1, 748.8 cm⁻¹.

LCMS (m/z): 590.1 (M+1, 95%).

### 3-[{4-(Biphenyl-2-ylamino)-6-[3-(2-methoxyphenyl)ureido]-[1,3,5]triazin-2-yl}-(3-fluorobenzyl)amino]propionic acid (Compound No. 114)

3-[{4-(Biphenyl-2-ylamino)-6-[3-(2-methoxyphenyl)ureido]-[1,3,5]triazin-2-yl}-(3-fluorobenzyl)amino]propionic acid was prepared by using 3-(3-fluorobenzyl)aminopropionic acid instead of 3-(4-methylbenzyl)aminopropionic acid in Step 3, Example 1.

m.p.: 115-128° C.

¹HNMR (TFA-d, 300 MHz): δ 7.45-8.40 (17H, m, aromatic), 5.61 (s) & 5.49 (s) [2H, NCH₂Ar], 4.76 (bs) & 4.58 (bs) [2H, NCH₂], 4.62 (s) & 4.60 (s) [3H, OCH₃] and 3.53 (bs) & 3.37 (bs), [2H, CH₂CO₂H].

IR (νₘₐₓ, KBr): 1701.1, 1600.1, 1250.2, 747.2 cm⁻¹.

LCMS (m/z): 608.5 (M+1, 100%).

### 3-[{4-(Biphenyl-2-ylamino)-6-[3-(2-methoxyphenyl)ureido]-[1,3,5]triazin-2-yl}-(3-chlorobenzyl)amino]propionic acid (Compound No. 115)

3-[{4-(Biphenyl-2-ylamino)-6-[3-(2-methoxyphenyl)ureido]-[1,3,5]triazin-2-yl}-(3-chlorobenzyl)amino]propionic acid was prepared by 3-(3-chlorobenzyl)aminopropionic acid instead of 3-(4-methyl benzyl)aminopropionic acid in Step 3, Example 1.

m.p.: 212-214° C.

¹HNMR (TFA-d, 300 MHz): δ 7.70-8.30 (14H, m, aromatic), 5.59 (s) & 5.46 (s) [2H, NCH₂Ar], 4.75 (bs) & 4.52 (bs) [2H, NCH], 4.61 (s) & 4.59 (s) [3H, OCH₃] and 3.52 (bs) & 3.36 (bs) [2H, CH₂CO₂H].

IR (νₘₐₓ, KBr): 1708.7, 1535.2, 1421.2, 747.1 cm⁻¹.

LCMS (m/z): 624.2 (M+1, 30%).

### 3-[{4-(Biphenyl-2-ylamino)-6-[3-(2-methoxyphenyl)ureido][1,3,5]triazin-2-yl}-(2-trifluoromethylbenzyl)amino]propionic acid (Compound No. 116)

3-[{4-(Biphenyl-2-ylamino)-6-[3-(2-methoxyphenyl)ureido][1,3,5]triazin-2-yl}-(2-trifluoromethylbenzyl)amino]propionic acid was prepared by using 3-(2-trifluoromethylbenzyl)aminopropionic acid instead of 3-(4-methylbenzyl)aminopropionic acid in Step 3, Example 1.

m.p.: 216-219° C.

¹HNMR (TFA-d, 300 MHz): δ 7.5-8.41 (17H, m, aromatic), 5.80 (s) & 5.73 (s) [2H, NCH₂Ar], 4.70 (bs) & 4.47 (bs) [2H, NCH₂], 4.57 (s) & 4.51 (s) [3H, OCH₃] and 3.50 (t), 3.33 (t) [2H, CH₂CO₂H].

IR (νₘₐₓ, KBr): 1712.6, 1609.8, 1313.2, 1119.4, 747.1 cm⁻¹.

LCMS (m/z): 658.1 (M+1, 100%).

### 3-[{4-(Biphenyl-2-ylamino)-6-[3-(2-methoxyphenyl)ureido]-[1,3,5]triazin-2-yl}-(3-trifluoromethylbenzyl)amino]propionic acid (Compound No. 117)

3-[{4-(Biphenyl-2-ylamino)-6-[3-(2-methoxyphenyl)ureido]-[1,3,5]triazin-2-yl}-(3-trifluoromethylbenzyl)amino]propionic acid was prepared by using 3-(3-trifluoromethylbenzyl)aminopropionic acid instead of 3-(4-methylbenzyl)aminopropionic acid in Step 3, Example 1.

m.p.: 114-126° C.

¹HNMR (TFA-d, 300 MHz): δ 7.70-8.30 (17H, m, aromatic), 5.64 (s) & 5.52 (s) [2H, NCH₂Ar], 4.73 (t, J=6 Hz) & 4.49 (t) [2H, NCH₂], 4.57 (s) & 4.55 (s) [3H, OCH₃] and 3.49 (t) & 3.33 (t) [2H, CH₂CO₂H]

IR (ν_{max,} KBr): 1710.4, 1536.1, 1329.2, 1123.5, 747.5 cm⁻¹.

LCMS (m/z): 658.1 (M+1, 100%).

### 3-[{4-(Biphenyl-2-ylamino)-6-[3-(2-methoxyphenyl)ureido]-[1,3,5]triazin-2-yl}-(3,5-bis-trifluoromethylbenzyl)amino]propionic acid (Compound No. 118)

3-[{4-(Biphenyl-2-ylamino)-6-[3-(2-methoxyphenyl)ureido]-[1,3,5]triazin-2-yl}-(3,5-bis-trifluoromethylbenzyl)amino]propionic acid was prepared by using 3-(3,5-bis trifluoromethylbenzyl)aminopropionic acid instead of 3-(4-methylbenzyl)aminopropionic acid in Step 3, Example 1.

m.p.: 132-135° C.

¹HNMR (TFA-d, 300 MHz): δ 7.05-8.00 (16H, m, aromatic), 5.11 (s) & 4.98 (s) [2H, NCH₂Ar], 4.13 (t, J=9 Hz) & 3.88 (t) [2H, NCH₂], 3.96 (s) & 3.94 (s), [3H, OCH₃] and 2.93 (t) & 2.74 (t) [2H, CH₂CO₂H].

IR (νₘₐₓ, KBr): 1709.8, 1537.5, 1279.5, 1173.6, 1133.9 cm⁻¹.

LCMS (m/z): 726.4 (M+1, 75%).

### 3-[{4-(Biphenyl-2-ylamino)-6-[3-(2-methoxyphenyl)ureido]-[1,3,5]triazin-2-yl}-(biphenyl-4-ylmethyl)amino]propionic acid (Compound No. 119)

3-[{4-(Biphenyl-2-ylamino)-6-[3-(2-methoxyphenyl)ureido]-[1,3,5]triazin-2-yl}-(biphenyl-4-ylmethyl)amino]propionic acid was prepared by using 3-(biphenyl-4-ylmethyl)aminopropionic acid instead of 3-(4-methylbenzyl)aminopropionic acid in Step 3, Example 1.

m.p.: 144-147° C.

¹HNMR (TFA-d, 300 MHz): δ 7.05-7.70 (22H, m, aromatic), 5.00 (s) & 4.87 (s) [2H, NCH₂Ar], 4.15 (t) & 4.03 (t) [2H, NCH₂], 3.95 (s) & 3.92 (s) [3H, OCH₃] and 2.90 (bs) & 2.72 (bs) [2H, CH₂CO₂H].

IR (νₘₐₓ, KBr): 1707.9, 1602.5, 1275.8, 748.6 cm⁻¹.

LCMS (m/z): 726.4 (M+1, 75%).

### 3-[{4-(Biphenyl-2-ylamino)-6-{3-(2-methoxyphenyl)ureido]-[1,3,5]triazin-2-yl}-(naphthalen-1-ylmethyl)amino]propionic acid (Compound No. 120)

3-[{4-(Biphenyl-2-ylamino)-6-{3-(2-methoxyphenyl)ureido]-[1,3,5]triazin-2-yl}-(naphthalen-1-ylmethyl)amino]propionic acid was prepared by reacting 3-(naphthalen-1-ylmethyl)aminopropionic acid instead of 3-(4-methylbenzyl)aminopropionic acid in Step 3, Example 1.

m.p.: 218-221° C.

¹HNMR (TFA-d, 300 MHz): δ 7.00-8.00 (20H, m, aromatic), 5.42 (s) & 5.35 (s) [2H, NCH₂Ar], 4.10 (bs) & 3.98 (bs) [2H, NCH₂], 3.95 (s) & 3.88 (s) [3H, OCH₃] and 2.70 (bs) & 2.52 (bs) [2H, CH₂CO₂H]

IR (νₘₐₓ, KBr): 1705.9, 1599.5, 1532.4, 809.7, 748.4 cm⁻¹.

LCMS (m/z): 640.5 (M+1, 10%).

### 3-[{4-(Biphenyl-2-ylamino)-6-[3-(2-methoxyphenyl)ureido]-[1,3,5]triazin-2-yl}-(2-pyridin-2-ylethyl)amino]propionic acid (Compound No. 121)

3-[{4-(Biphenyl-2-ylamino)-6-[3-(2-methoxyphenyl)ureido]-[1,3,5]triazin-2-yl}-(2-pyridin-2-ylethyl)amino]propionic acid was prepared by using 3-(2-pyridin-2-ylethyl)aminopropionic acid instead of 3-(4-methylbenzyl)aminopropionic acid in Step 3, Example 1.

m.p.: 225-227° C.

¹HNMR (TFA-d, 300 MHz): δ 7.65-9.40 (17H, m, aromatic), 4.70 (4H, m, NCH₂×2), 4.57 (3H, s, OCH₃), 4.20 (bs) & 4.08 (bs) [2H, Ar CH₂] and 3.56 (bs) & 3.44 (bs) [2H, CH₂CO₂H].

IR (νₘₐₓ, KBr): 1690.3, 1542.1, 1324.4, 811.5, 746.1 cm⁻¹.

LCMS (m/z): 605.2 (M+1, 100%).

### 1-{4-(Biphenyl-2-ylamino)-6-[3-(2-methoxyphenyl)ureido]-[1,3,5]triazin-2-yl}-pyrrolidine-2S-carboxylic acid (Compound No. 125)

1-{4-(Biphenyl-2-ylamino)-6-[3-(2-methoxyphenyl)ureido]-[1,3,5]triazin-2-yl}-pyrrolidine-2S-carboxylic acid was prepared by using pyrrolidin-2S-carboxylic acid (L-proline) instead of 3-(4-methylbenzyl)aminopropionic acid in Step 3, Example 1.

m.p.: 175-180° C.

¹HNMR (TFA-d, 300 MHz): δ 7.94-8.40 (m) and 7.65 (m) [13H, aromatic], 5.41 (s) & 5.25 (s) [1H, NCHCO₂H], 4.67 (s) & 4.53 (m) [5H, OCH₃ and NCH₂] and 3.13 (1H, m) & 2.86 (3H, m) [CH₂×2 (ring)].

IR (νₘₐₓ, KBr): 1719.0, 1657.2, 1523.1, 1202.1, 749.9 cm⁻¹.

LCMS (m/z): 526.1 (M+1, 100%).

### 1-{4-(Biphenyl-2-ylamino)-6-[3-(2-methoxyphenyl)ureido]-[1,3,5]triazin-2-yl}-thiazolidine-4-carboxylic acid (Compound No. 126)

1-{4-(Biphenyl-2-ylamino)-6-[3-(2-methoxyphenyl)ureido]-[1,3,5]triazin-2-yl}-thiazolidine-4-carboxylic acid was synthesized by using thiazolidine-4-carboxylic acid instead of 3-(4-methyl benzyl)aminopropionic acid in Step 3, Example 1.

m.p.: 170-175° C.

¹HNMR (TFA-d, 300 MHz): δ 7.75-8.21 (m), 7.58 (m) [16H, aromatic and 3NH], 5.75 (s) & 5.54 (s) [NCHCO₂H], 5.36 (2H, AB_{q}, J=9 Hz, NCH₂S), 4.47 (3H, s, OCH₃) and 3.9-4.11 (2H, m, SCH₂).

LCMS (m/z): 544.1 (M+1, 100%).

### 1-{4-(Biphenyl-2-ylamino)-6-[3-(2-methoxyphenyl)ureido]-[1,3,5]triazin-2-yl}-piperidine-3-carboxylic acid (Compound No. 127)

1-{4-(Biphenyl-2-ylamino)-6-[3-(2-methoxyphenyl)ureido]-[1,3,5]triazin-2-yl}-piperidine-3-carboxylic acid was prepared by using piperidine-3-carboxylic acid instead of 3-(4-methyl benzyl)aminopropionic acid in Step 3, Example 1.

m.p.: 245-247° C.

¹HNMR (TFA-d, 300 MHz): δ 8.19 (1H, bs), 7.77-8.00 (10H, m) and 7.55 (2H, m) [aromatic], 4.95 (1H, m) & 4.70 (1H, m) [NCH₂], 4.40 (3H, s, OCH₃), 4.22 (1H, m) & 4.03 (1H, bs) [NCH₂], 3.29 (1H, s, CHCO₂H) and 2.75 (1H, m), 2.49 (2H, m) & 2.21 (1H, m) [CH₂×2 (ring)].

IR (νₘₐₓ, KBr): 1703.3, 1574.4, 1337.6, 1276.3, 746.1 cm⁻¹.

LCMS (m/z): 540.1 (M+1, 100%).

### 1-{4-(Biphenyl-2-yl-amino)-6-[3-(2-methoxyphenyl)ureido]-[1,3,5]triazin-2-yl}-piperidine-4-carboxylic acid. (Compound No. 128)

1-{4-(Biphenyl-2-yl-amino)-6-[3-(2-methoxyphenyl)ureido]-[1,3,5]triazin-2-yl}-piperidine-4-carboxylic acid was prepared by using piperidine-4-carboxylic acid instead of 3-(4-methyl benzyl)aminopropionic acid in Step 3, Example 1.

m.p.: 130-135° C.

¹HNMR (TFA-d, 300 MHz): δ 7.76-8.15 (11H, m), 7.59 (2H, bs) [aromatic], 5.15 (2H, m, NCH₂), 4.61 (s) & 4.46 (s) [3H, OCH₃], 3.83 (2H, m, NCH₂), 2.69 (1H, m, CHCO₂H) and 2.39 (2H, m) & 1.90 (2H, m) [CH₂×2 (ring)].

IR (νₘₐₓ, KBr): 1705.0, 1602.5, 1528.9, 1278.6, 1028.0, 747.9 cm⁻¹

LCMS (m/z): 540.0 (M+1, 100%).

### 2-{4-(Biphenyl-2-ylamino)-6-[3-(2-methoxyphenyl)ureido]-[1,3,5]triazin-2-yl}-1,2,3,4-tetrahydro-isoquinoline-3-carboxylic acid (Compound No. 129)

2-{4-(Biphenyl-2-ylamino)-6-[3-(2-methoxyphenyl)ureido]-[1,3,5]triazin-2-yl}-1,2,3,4-tetrahydro-isoquinoline-3-carboxylic acid was prepared by using 1,2,3,4-tetrahydro-isoquinoline-3-carboxylic acid in place of 3-(4-methylbenzyl)aminopropionic acid in Step 3, Example 1.

m.p.: 138-145° C.

¹HNMR (TFA-d, 300 MHz): δ 7.60-8.07 (17H, m, aromatic), 5.93 (1H, m) & 5.69 (1H, bs) [NCHCO₂H], 5.40-5.50 (2H, m, NCH₂), 4.48 (s) & 4.46 (s) [3H, OCH₃] and 3.90 (2H, m, ArCH₂).

IR (νₘₐₓ, KBr): 1702.9, 1599.9, 1530.8, 1428.1, 1320 cm⁻¹.

LCMS (m/z): 588.3 (M+1, 100%).

### 1-{4-(Biphenyl-2-ylamino)-6-[3-(2-methoxyphenyl)ureido]-[1,3,5]triazin-2-yl}piperidine-2-carboxylic acid (Compound No. 130)

1-{4-(Biphenyl-2-ylamino)-6-[3-(2-methoxyphenyl)ureido]-[1,3,5]triazin-2-yl}piperidine-2-carboxylic acid was prepared by using piperidine-2-carboxylic acid instead of 3-(4-methylbenzyl)amino-propionic acid in Step 3, Example 1.

m.p.: 150-154° C.

¹H NMR (TFA-d, 300 MHz): δ 7.49-7.80 (1H, m) & 7.17 (2H, bs) [aromatic], 5.74 (bs) & 5.58 (bs) [1H, NCH], 4.78 (1H, m) & 3.35 (1H, m) [NCH₂], 4.05 (3H, s, OCH₃) and 2.60 (1H, m), 2.00 (3H, m) & 1.67 (2H, m) [CH₂×3].

LCMS (m/z): 540.3 (M+1, 100%)

### Tris salt of 3-[{4-(Biphenyl-2-ylamino)-6-[3-(2-methoxyphenyl)ureido]-[1,3,5]triazin-2-yl}-(3-methylbenzyl)amino]propionic acid (Compound No. 48)

To Compound No. 31 (100 mg, 0.16 mmol) in dry THF (5 ml) was added tris (hydroxymethyl)aminomethane (9 mg, 0.16 mmol) and the reaction mixture was stirred overnight at room temperature. Solvents were evaporated to dryness to afford the title compound.

m.p.: 85-92° C.

¹HNMR (TFA-d, 300 MHz): δ 8.05-8.75 (15H, m, aromatic), 6.04 (s) & 5.93 (s) [2H, NCH₂Ar], 5.31 (6H, s, CH₂OH×3), 5.21 (2H, s, NCH₂), 5.07 (s) & 5.05 (s) [3H, OCH₃], 3.95 (2H, m, CH₂CO₂H) and 3.54 (s) & 3.48 (s) [3H, Ar CH₃].

IR (νₘₐₓ, KBr): 1670.5, 1510.2, 1249.4, 1048.0, 747.3 cm⁻¹

LCMS (m/z): 122.2 (87%), 604.2 (100%)

### Na salt of 3-[{4-(Biphenyl-2-ylamino)-6-[3-(2-methoxyphenyl)-ureido]-[1,3,5]triazin-2-yl}-(3-methylbenzyl)amino]propionic acid (Compound No. 49)

To Compound No. 31 (100 mg, 0.16 mmol) in dry THF:MeOH (2:1 ml) was added sodium methoxide (20 mg, 0.16 mmol) and the reaction mixture stirred at room temperature for 5 hours. Evaporation of the solvents to dryness afforded the title compound.

m.p.: 122-135° C.

¹HNMR (TFA-d, 300 MHz): δ 8.0-8.56 (19H, m, aromatic and 2NH), 6.01 (s) & 5.91 (s) [2H, NCH₂Ar], 5.11 (m, NCH₂), 5.04 (s) & 5.02 (s) [OCH₃], 3.92 (s) & 3.75 (s) [2H, CH₂CO₂H] and 3.45 (s) & 3.42 (s) [3H, ArCH₃].

IR (νₘₐₓ, KBr): 1692.2, 1599.7, 1535.4, 1248.9, 746.7 cm⁻¹

LCMS (m/z): 604.2 (M−Na)+2, 100%), 626.5 (M+1, 30%)

### Example 2

### Synthesis of 1-{4-(Biphenyl-2-ylamino)-[1,3,5]triazin-2-yl}-3-(2-methoxyphenyl)urea (Compound No. 18)

To the chlorotriazine (260 mg, 0.58 mmol) as obtained from Step 2, Example 1, in tetrahydrofuran (50 ml) was added triethylamine (1 ml) and 10% Pd/C (dry, 100 mg) and the mixture was taken under H₂ atmosphere at 50 psi for 5 hours. The solids were filtered through a celite pad and the filtrate was concentrated and residue dried under vacuum to obtain the title compound (130 mg, 54%) as a solid.

m.p.: 219° C.

¹HNMR (DMSO-d₆, 300 MHz): δ 11.45 (1H, bs, NH), 9.95 (1H, s, NH), 9.49 (1H, bs, NH), 8.38 (1H, s, triazine H), 8.01 (1H, bs), 7.31-7.50 (9H, m) and 6.89-7.03 (2H, m) [aromatic] and 3.76 (3H, s, OCH₃).

IR (νₘₐₓ, KBr): 1696.4, 1548.9, 1252.2, 744.5 cm⁻¹.

LCMS (m/z): 413.0 (M+1, 45%).

### Example 3

### Synthesis 1-{4-(Biphenyl-2-ylamino)-6-methoxy-[1,3,5]triazin-2-yl}-3-(2-methoxyphenyl)urea (Compound No. 19)

To the chlorotriazine (211 mg, 0.5 mmol) prepared as in Step 2, Example 1, in tetrahydrofuran (9 ml) and methanol (1 ml) was added sodium methoxide (41 mg, 0.75 mmol). The reaction mixture was refluxed for 3 hour and poured over cold water (25 ml). It was extracted with ethyl acetate (2×25 ml) and the organic layer washed with water and brine and dried over anhydrous sodium sulfate. Evaporation of the solvent followed by purification of the residue over a silica gel column using 40% EtOAc-hexane as eluent furnished the title compound (110 mg, 53%) as a solid.

m.p.: 202° C.

¹HNMR (DMSO-d₆, 300 MHz): δ 10.6 (1H, bs, NH), 9.86 (1H, bs, NH), 7.97 (1H, bs, NH), 7.49 (1H, d, J=7.2 Hz), 7.28-7.38 (9H, m) & 6.86-7.03 (3H, m) [aromatic] and 3.81 (3H, s) & 3.68 (3H, s) [OCH₃×2].

IR (νₘₐₓ, KBr): 1684.7, 1420.6, 816.5, 747.1 cm⁻¹.

LCMS (m/z): 443.0 (M+1, 100%).

### Example 4

### Synthesis of 3-{(3-Fluorobenzyl)-[4-[3-(2-methoxyphenyl)ureido]-6-(naphthalen-2-yloxy)-[13,5]triazin-2-yl]amino}propionic acid (Compound No. 105)

### Step 1: Synthesis of 4-Chloro-6-(naphthalen-2-yloxy)-[1,3,5]triazin-2-ylamine

A solution of β-naphthol (8.78 g, 60 mmol) in acetone (100 ml) was added dropwise to a reaction mixture containing 2-amino-4,6-dichloro-1,3,5-triazine (10 g, 60 mmol) and potassium carbonate (8.4 g, 60 mmol) in acetone (100 ml) maintained at room temperature. The reaction mixture was stirred at room temperature for 16 h and then refluxed for 6 hours. After cooling, the reaction mixture was poured over ice and the preciptated solid filtered, washed water and dried under vacuum. The crude solid was then washed with a solution of 10% CH₂Cl₂-hexane (100 ml) and further dried under vacuum to obtain the title compound (15.4 g, 73%) as a white solid.

¹HNMR (DMSO-d₆, 300 MHz): δ 8.05-8.15 (3H, m), 8.01 (2H, bs, NH₂), 7.77 (1H, s), 7.55 (2H, m) and 7.39 (1H, m) [aromatic].

IR (νₘₐₓ, KBr): 1696.2, 1528.4, 1379.9, 800.5 cm⁻¹.

LCMS (m/z): 273.0 (M+1, 100%).

### Step 2: Synthesis of 1-[4-Chloro-6-(naphthalen-2-yloxy)-[1,3,5]triazin-2-yl]-3-(2-methoxyphenyl)urea (Compound No. 1)

To the amino triazine (15.1 g, 55 mmol) obtained from Step 1, Example 4, in THF (200 ml) at 0° C. was added sodium hydride (2.64 g, 50% dispersion in oil, 55 mmol) followed immediately by 2-methoxyphenyl isocyanate (8.3 g, 55 mmol)). The reaction mixture was stirred at 0° C. for 30 min and at room temperature for 12 h. The reaction mixture was poured into ice-water and extracted with ethyl acetate (2×250 ml). The organic layer were combined, washed with brine and dried (Na₂SO₄). Evaporation of the eluent followed by purification of the residue over a silica gel column using 40% EtOAc:Hexane afforded the title compound (14.1 g, 60%) as a white solid.

¹HNMR (DMSO-d₆, 300 MHz): δ 11.12 (1H, s) and 11.08 (1H, s) [NH×2], 8.14 (1H, d, J=7.8 Hz), 7.9-8.05 (3H, m), 7.82 (1H, bs), 7.4-7.6 (3H, m), 7.04 (2H, bs) and 6.91-6.96 (1H, m) [aromatic] and 3.81 (3H, s, OCH₃).

IR (νₘₐₓ, KBr): 1707.1, 1289, 808, 744 cm⁻¹.

LCMS (m/z): 422.2 (M+1, 70%), 424 (M+3, 30%).

### Step 3: Synthesis of 3-{(3-Fluorobenzyl)-[4-[3-(2-methoxyphenyl)ureido]-6-(naphthalen-2-yloxy)-[1,3,5]triazin-2-yl]amino}propionic acid

Following the general procedure as mentioned for Example 1, the title compound was prepared from the Li salt of the aminopropionic acid (obtained by the hydrolysis of ethyl 3-(3-fluorobenzyl)aminopropionate (106 mg, 0.47 mmol using LiOH in H₂O (20 mg, 0.47 mmol) and the chlorotriazine (200 mg, 0.47 mmol)) obtained from Step 2 above. Column chromatography of the crude residue afforded the pure title compound (114 mg, 41%).

m.p.: 223° C.

¹HNMR (TFA-d, 300 MHz): δ 7.35-8.70 (15H, m, aromatic), 5.71 (s) & 5.37 (s) [2H, NCH₂Ar], 4.84 (t) & 4.40 (t) [2H, NCH₂] 4.69 (s) & 4.66 (s) [3H, OCH₃] and 3.61 (t) & 3.20 (t) [2H, CH₂CO₂H].

IR (νₘₐₓ, KBr): 1715.6, 1596.6, 1254.1, 748.1 cm⁻¹.

LCMS (m/z): 583.4 (M+1, 90%).

The following compounds were prepared similarly:

### 1-[4-Chloro-6-(nalphthalen-2-yloxy)-[1,3,5]triazin-2-yl]-3-(2-chlorophenyl) urea (Compound No. 2)

1-[4-Chloro-6-(naphthalen-2-yloxy)-[1,3,5]triazin-2-yl]-3-(2-chlorophenyl) urea was prepared following the procedure of Step 2 Example 4, by using 2-chlorophenyl isocyanate in place of 2-methoxyphenyl isocyanate.

¹HNMR (DMSO-d₆, 300 MHz): δ 11.27 (1H, s) & 10.97 (1H, s) [NH×2], 8.16 (1H, d, J=7.8 Hz), 7.91-8.02 [3H, m], 7.86 (1H, bs), 7.51-7.57 (4H, m), 7.31 (1H, t, J=7.5 Hz) and 7.15 (1H, t, J=7.5 Hz) [aromatic].

IR (νₘₐₓ, KBr): 1717.0, 1426.4, 1237.1, 745.6 cm⁻¹.

LCMS (m/z): 426.1 (M+1, 80%), 428.1 (M+3, 50%).

### 1-[4-Chloro-6-(naphthalen-2-yloxy)-[1,3,5]triazin-2-yl]-3-(4-chlorophenyl) urea (Compound No. 3)

1-[4-Chloro-6-(naphthalen-2-yloxy)-[1,3,5]triazin-2-yl]-3-(4-chlorophenyl) urea was prepared following the procedure of Step 2 Example 4, by using 4-chlorophenyl isocyanate in place of 2-methoxyphenyl isocyanate.

¹HNMR (DMSO-d₆, 300 MHz): δ 11.19 (1H, s) & 9.99 (1H, s) [NH×2], 7.94-8.09 (4H, m), 7.55-7.65 (3H, m), 6.81 (2H, d, J=8.7 Hz) and 6.48 (2H, d, J=8.4 Hz) [aromatic].

IR (νₘₐₓ, KBr): 1714.5, 1695.1, 1538.9, 1237.8, 807.3 cm⁻¹. LCMS (m/z): 426 (M+1, 100%) and 428 (M+3, 50%).

### 1-[4-Chloro-6-(naphthalen-2-yloxy)-[1,3,5]triazin-2-yl]-3-(4-methoxyphenyl) urea (Compound No. 4)

1-[4-Chloro-6-(naphthalen-2-yloxy)-[1,3,5]triazin-2-yl]-3-(4-methoxyphenyl) urea was prepared by using 4-methoxyphenyl isocyanate in Step 2, Example 4 instead of 2-methoxyphenyl isocyanate.

¹HNMR (DMSO-d₆, 300 MHz): δ 11.04 (1H, s) & 9.76 (1H, s) [NH×2], 7.94-8.07 (4H, m), 7.53-7.64 (3H, m) and 6.34 (4H, s) [aromatic] and 3.62 (3H, s, OCH₃).

IR (νₘₐₓ, KBr): 1694.2, 1564.1, 1540.3, 1227.6 cm⁻¹.

LCMS (m/z): 422.0 (M+1, 100%) 424 (M+3, 23%).

### [4-[3-(2-Methoxyphenyl)ureido]-6-naphthalen-2-yloxy)-[1,3,5]triazin-2-yl]aminoacetic acid (Compound No. 7)

4-[3-(2-Methoxyphenyl)ureido]-6-naphthalen-2-yloxy)-[1,3,5]triazin-2-yl]aminoacetic acid. prepared by using aminoacetic acid (glycine) instead of 3(3-fluorobenzyl)aminopropionic acid in Step 3, Example 4.

¹HNMR (DMSO-d₆, 300 MHz): δ 11.49 (s) & 11.21 (s) [1H, NH], 10.15 (s) & 10.08 (s) [1H, NH] 7.93-8.15 (4H, m), 7.81 (1H, d, J=5.3 Hz), 7.44-7.54 (3H, m) and 6.92-7.03 (3H, m) [aromatic], 4.02 (1H, bs) & 3.72 (1H, bs) [NCH₂] and 3.86 (3H, s, OCH₃).

IR (νₘₐₓ, KBr): 1711.5, 1546.0, 1242.7, 748.1 cm⁻¹.

LCMS (m/z): 461.0 (M+1, 100%).

### [4-[3-(2-Chlorophenyl)ureido]-6(naphthalen-2-yloxy)-[1,3,5]triazin-2-yl]aminoacetic acid (Compound No. 8)

[4-[3-(2-Chlorophenyl)ureido]-6(naphthalen-2-yloxy)-[1,3,5]triazin-2-yl]aminoacetic acid was synthesized following the procedure of Example 4, by using 2-chlorophenyl isocyanate instead of 2-methoxyphenyl isocyanate in Step 2 and aminoacetic acid (glycine) instead of 3-(3-fluorobenzyl)aminopropionic acid in Step 3, Example 4.

¹HNMR (DMSO-d₆, 300 MHz): δ 11.46 (s) & 11.29 (s) [1H, NH], 10.32 (s) & 10.25 (s) [1H, NH] 7.77-7.95 (5H, m), 7.52 (1H, d, J=2.7 Hz) and 7.12-7.53 (6H, m) [aromatic and 1 NH] and 3.90 (1H, d, J=5.7 Hz) & 3.72 (1H, d, J=5.7 Hz) [NCH₂].

IR (νₘₐₓ, KBr): 1708.0, 1345.8, 1240.1, 746.2 cm⁻¹.

LCMS (m/z): 465.0 (M+1, 100%), 467.3 (M+3, 30%).

### [4-[3-(2-Methylphenyl)ureido]-6-(naphthalen-2-yloxy)-[1,3,5]triazin-2-yl]aminoacetic acid (Compound No. 9)

[4-[3-(2-Methylphenyl)ureido]-6-(naphthalen-2-yloxy)-[1,3,5]triazin-2-yl]aminoacetic acid was prepared by using 2-methylphenyl isocyanate in Step 2 and aminoacetic acid (glycine) in Step 3 of Example 4 in place of 2-methoxyphenyl isocyanate Step 2 and 3(3-fluorobenzyl)aminopropionic acid in Step 3, Example 4.

¹HNMR (DMSO-d₆, 300 MHz): δ 10.84 (s) & 10.76 (s) [1H, NH], 10.09 (s) & 9.99 (s) [1H, NH], 7.78-7.98 (4H, m), 7.39-7.55 (4H, m) and 7.00-7.17 (3H, m) [aromatic] 3.77 (1H, bs) & 3.67 (1H, d, J=4.5 Hz) [NCH₂] and 1.85 (s) & 1.99 (s) [3H, ArCH₃].

IR (νₘₐₓ, KBr): 1703.2, 1590.0, 1241.9, 747.5 cm⁻¹.

LCMS (m/z): 445.2 (M+1, 100%).

### [4-[3-(4-Methoxyphenyl)ureido]-6-(naphthalen-2-yloxy)-[1,3,5]triazin-2-yl]aminoacetic acid (Compound No. 10)

[4-[3-(4-Methoxyphenyl)ureido]-6-(naphthalen-2-yloxy)-[1,3,5]triazin-2-yl]aminoacetic acid was prepared by taking Compound No. 4 and aminoacetic acid (glycine) as in Step 3, Example 4.

¹HNMR (DMSO-d₆, 300 MHz): δ 10.69 (bs) & 10.62 (bs) [1H, NH], 9.97 (s), 9.88 (s) & 9.79 (s) [2H, NH×2], 7.46-8.07 (8H, m), 6.34-7.43 (3H, m) [aromatic] and 3.62-3.72 (5H, MOCH₃ and NCH₂).

IR (νₘₐₓ, KBr): 1694.9, 1546.0, 1244.0, 808.2 cm⁻¹.

LCMS (m/z): 461.0 (M+1, 100%).

### [4-[3-(4-Chlorophenyl)ureido]-6-(naphthalen-2-yloxy-[1,3,5]triazin-2-yl]aminoacetic acid (Compound No. 11)

[4-[3-(4-Chlorophenyl)ureido]-6-(naphthalen-2-yloxy)-[1,3,5]triazin-2-yl]aminoacetic acid was prepared by using Compound No. 3 and aminoacetic acid (glycine) as in Step-3, Example 4 instead of 3-(3-fluorobenzyl)aminopropionic acid.

¹HNMR (DMSO-d₆, 300 MHz): δ 10.95 (1H, bs, NH), 10.06-10.14 (1H, NH), 6.61-8.01 (12H, m, 11H aromatic & 1 NH) and 3.75 (2H, bs, NCH₂).

IR (νₘₐₓ, KBr): 1698.4, 1593.1, 1241.5, 810.9 cm⁻¹.

LCMS (m/z): 465.0 (M+1, 100%)

### [4-[3-(4-Methylphenyl)ureido]-6-(naphthalen-2-yloxy]-[1,3,5]triazin-2-yl]aminoacetic acid (Compound No. 12)

[4-[3-(4-Methylphenyl)ureido]-6-(naphthalen-2-yloxy]-[1,3,5]triazin-2-yl]aminoacetic acid was prepared by using 4-methylphenyl isocyanate in Step 2 and aminoacetic acid (glycine) in Step 3, Example 4 instead of 2-methoxyphenyl isocyanate (Step 2) and 3-(3-fluorobenzyl)aminopropronic acid (Step 4).

¹HNMR (DMSO-d₆, 300 MHz): δ 10.89 (s) & 10.72 (s) [1H, NH], 9.86 (bs) & 9.64 (bs) [1H, NH], 6.74-7.97 (12H, m, aromatic and 1NH] and 2.24 (s) & 2.14 (s) [3H, ArCH₃]

IR (νₘₐₓ, KBr): 1699.5, 1595.1, 1407.2, 1246.1, 812.0 cm⁻¹.

LCMS (m/z): 445.2 (M+1, 100%).

### 2S-{4-[3-(2-Methoxyphenyl)-ureido]-6-(naphthalen-2-yloxy)-[1,3,5]triazin-2-ylamino}-3-phenylpropionic acid (Compound No. 13)

2S-{4-[3-(2-Methoxyphenyl)-ureido]-6-(naphthalen-2-yloxy)-[1,3,5]triazin-2-ylamino}-3-phenylpropionic acid was prepared by using 2-amino-3-phenylpropionic acid (L-phenylalanine) instead of 3-(3-fluorobenzyl)aminopropionic acid in Step 3, Example 4.

m.p.: 202° C.

¹HNMR (DMSO-d₆, 300 MHz): δ 11.42 (s) & 11.15 (s) [1H, NH], 10.07 (s) & 10.05 (s) [1H, NH], 7.79-8.01 (5H, m), 7.15-7.54 (4H, m) and 6.90-7.01 (8H, m) [aromatic], 4.82 (bs) & 4.35 (bs) [1H, NCH], 3.76 (s) & 3.73 (s) [3H, OCH₃] and 2.8-3.5 (2H, m, CH₂ Ar).

IR (νₘₐₓ, KBr): 1697.1, 1404.4, 1341.3, 1244.5, 744.0 cm⁻¹.

LCMS (m/z): 551.2 (M+1, 70%).

### 3-{4-[3-(2-Methoxyphenyl)ureido]-6-(naphthalen-2-yloxy)-[1,3,5]triazin-2-yl amino}3-phenylpropionic acid (Compound No. 14)

3-{4-[3-(2-Methoxyphenyl)ureido]-6-(naphthalen-2-yloxy)-[1,3,5]triazin-2-yl amino}3-phenylpropionic acid was prepared by using 3-amino-3-phenylpropionic acid instead of 3-(3-fluorobenzyl)aminopropionic acid in Step 3, Example 4.

m.p.: 149° C.

¹HNMR (DMSO-d₆, 300 MHz): δ 11.46 (s) & 10.31 (s) [1H, NH], 10.12 (s) & 10.02 (s) [1H, NH], 8.59 (d) & 8.45 (d) [1H, NH], 6.93-8.3 (16H, m, aromatic), 5.46 (m) & 5.12 (m) [1H, NCH], 3.83 (s) & 3.81 (s) [3H, OCH₃] and 2.2-2.94 (2H, m, CH₂CO₂H).

IR (νₘₐₓ, KBr): 1696.8, 1243.8, 747.9 cm⁻¹.

LCMS (m/z): 551.2 (M+1, 25%).

### 3-{Benzo[1,3]dioxo-5-yl-[4-[3-(2-methoxyphenyl)ureido]-6-napthalen-2-yloxy)-[1,3,5]triazin-2-ylamino)propionic acid ethyl ester (Compound No. 15)

3-{Benzo[1,3]dioxo-5-yl-[4-[3-(2-methoxyphenyl 1,3,5]triazin-2-ylamino)propionic acid ethyl ester. (Compound No. 15) was prepared by reacting 3-amino-3-(benzo[1,3]dioxo-5-yl)propionic acid ethyl ester instead of 3-(3-fluorobenzyl)aminopropionic acid in Step 3, Example 4.

m.p.: 222° C.

¹HNMR (DMSO-d₆, 300 MHz): δ 11.55 (s) & 11.44 (s) [1H, NH], 10.25 (s) & 10.10 (s) [1H, NH], 8.54 (d, J=8.1 Hz) & 8.44 (d, J=8.1 Hz) [1H, NH], 8.02-8.15 (4H, m), 7.87 (1H, s), 7.62 (2H, bs), 7.50 (1H, t, J=8.1 Hz), 7.11 (1H, d, J=4.8 Hz), 7.01 (1H, bs), 6.84 (1H, m) and 6.62 (d) & 6.34 (d) [1H] [aromatic], 6.02 (2H, s, OCH₂O), 5.47 (m) & 5.02 (m) [1H, NCH], 3.8-4.09 (2H, m, OCH₂), 3.89 (3H, s, OCH₃), 2.5-3.0 (2H, m, CH₂CO) and 1.06-1.17 (3H, m, CH₃).

IR (νₘₐₓ, KBr): 1737.6, 1691.7, 1595.6, 1558.8, 1241.6 cm⁻¹.

LCMS (m/z): 623.1 (M+1, 100%).

### 3-{Benzo[1,3]dioxo-5-yl-([4-[3-(2-methoxyphenyl)-ureido]-6-napthalen-2-yloxy)-[1,3,5]triazin-2-ylamino}propionic acid (Compound No. 16)

3-{Benzo[1,3]dioxo-5-yl-([4-[3-(2-methoxyphenyl)-ureido]-6-napthalen-2-yloxy)-[1,3,5]triazin-2-ylamino}propionic acid was prepared by using 3-amino-3-(benzo-[1,3]-dioxo-5-yl)propionic acid instead of 3-(3-fluorobenzyl) aminopropionic acid in Step 3, Example 4.

m.p.: 221° C.

¹HNMR (DMSO-d₆, 300 MHz): δ 11.46 (s) & 11.38 (s) [1H, NH], 10.16 (s) & 10.03 (s) [1H, NH], 8.47 (d) & 8.41 (d) [1H, NH], 7.92-8.07 (4H, m), 7.79 (1H, d, J=9.2 Hz), 7.55 (2H, t, J=3.9 Hz), 7.31-7.55 (5H, m), 6.90-7.10 (4H, m), 6.6-6.90 (2H, m) and 6.53 (d) & 6.30 (d) [1H] [aromatic], 5.97 (s) & 5.94 (s) [2H, OCH₂O], 5.34 (m) & 4.91 (m) [NCH], 3.84 (s) & 3.82 (s) [3H, OCH₃] and 2.5-2.85 (2H, m, CH₂CO).

IR (νₘₐₓ, KBr): 1711.4, 1690.3, 1594.2, 1554.9, 1241.8, 747.5 cm⁻¹.

LCMS (m/z): 595.2 (M+1, 65%).

### 3-{4-[3-(2-Methoxyphenyl)ureido]-6-napthalen-2-yloxy)-[1,3,5]triazin-2-yl amino}propionic acid (Compound No. 43)

3-{4-[3-(2-Methoxyphenyl)ureido]-6-napthalen-2-yloxy)-[1,3,5]triazin-2-yl amino}propionic acid was prepared by using 3-aminopropionic acid (β-alanine) instead of 3-(3-fluorobenzyl)aminopropionic acid in Step 3, Example 4.

m.p.: 222-230° C.

¹HNMR (DMSO-d₆, 300 MHz): δ 12.1 (1H, bs, CO₂H), 11.45 (s) & 11.30 (s) [1H, NH], 10.10 (s) & 10.00 (s) [1H, NH], 7.78-8.10 (5H, m), 7.45-7.54 (3H, m) & 6.90-7.10 (3H, m) [aromatic], 3.86 (1H, t, J=6 Hz) & 3.53 (1H, t, J=3 Hz) [NCH₂], 3.80 (3H, s, OCH₃) and 2.47 (m, CH₂CO₂H).

IR (νₘₐₓ, KBr): 1690.1, 1600.8, 1549.5, 811.0, 747.4 cm⁻¹.

LCMS (m/z): 500.2 (M+1, 100%).

### 3-{(3-Methoxybenzyl)-[4-[3-(2-methoxyphenyl)ureido]-6-(nalphthalen-2-yloxy)-[1,3,5]triazin-2-yl]amino}propionic acid (Compound No. 44)

3-{(3-Methoxybenzyl)-[4-[3-(2-methoxyphenyl)ureido]-6-(naphthalen-2-yloxy)-[1,3,5]triazin-2-yl]amino}propionic acid was prepared by using 3-(3-methoxybenzyl)aminopropionic acid instead of 3-(3-fluorobenzyl)aminopropionic acid in Step 3, Example 4.

m.p.: 189-194° C.

¹HNMR (TFA-d, 300 MHz): δ 7.70-9.10 (17H, m, 15H aromatic and 2NH), 6.07 (s) & 5.79 (s) [2H, NCH₂Ar], 5.21 (1H, t) & 4.79 (1H, bs) [NCH₂], 5.06 (s), 5.03 (s), 4.97 (s) & 4.96 (s) [6H, OCH₃×2] and 3.96 (1H, s) & 3.56 (1H, s), [CH₂CO₂H].

IR (νₘₐₓ, KBr): 1716.3, 1597.8, 1535.5, 1253.5, 747.9 cm⁻¹.

LCMS (m/z): 595.1 (M+1, 100%).

### 3-{(4-Methoxybenzyl)-[4-[3-(2-methoxyphenyl)ureido]-6-(naphthalen-2-yloxy)-[1,3,5]triazin-2-yl]amino}propionic acid (Compound No. 45)

3-{(4-Methoxybenzyl)-[4-[3-(2-methoxyphenyl)ureido]-6-(naphthalen-2-yloxy)-[1,3,5]triazin-2-yl]amino}propionic acid was prepared by using 3-(4-methoxybenzyl)aminopropionic acid instead of 3-(3-fluorobenzyl)aminopropionic acid in Step 3, Example 4.

m.p.: 197-205° C.

¹HNMR (DMSO-d₆, 300 MHz): δ 12.1 (1H, bs, CO₂H), 11.26 (s) & 11.13 (s) [1H, NH], 10.19 (s) & 10.17 (s) [1H, NH], 7.20-8.05 (8H, m) & 6.65-7.10 (7H, m) [aromatic], 4.89 (s) & 4.42 (s) [2H, NCH₂Ar], 3.70-3.80 (2H, m, NCH₂), 3.72 (s) & 3.71 (s) [6H, OCH₃×2] and 2.40 (1H, m, CH₂CO₂H).

IR (νₘₐₓ, KBr): 1708.7, 1599.5, 1243.4, 1028.7, 750.5 cm⁻¹.

LCMS (m/z): 595.0 (M+1, 100%).

### 3-{(4-Fluorobenzyl)-[4-[3-(2-methoxyphenyl)-ureido]-6-(naphthalen-2-yloxy)-[1,3,5]triazin-2-yl]amino}propionic acid (Compound No. 46)

3-{(4-Fluorobenzyl)-[4-[3-(2-methoxyphenyl)-ureido]-6-(naphthalen-2-yloxy)-[1,3,5]triazin-2-yl]amino}propionic acid was prepared by using 3-(4-fluorobenzyl)aminopropionic acid instead of 3-(3-fluorobenzyl)aminopropionic acid in Step 3, Example 4.

m.p.: 211-217° C.

¹HNMR (TFA-d, 300 MHz): δ 7.83-9.10 (15H, m, aromatic), 6.05 (s) & 5.69 (s) [2H, NCH₂Ar], 5.19 (1H, bs) & 4.72 (1H, bs) [NCH₂], 5.04 (s) & 5.02 (s) [3H, OCH₃] and 3.95 (s) & 3.52 (s) [CH₂CO₂H].

IR (νₘₐₓ, KBr): 1714.1, 1603.9, 1242.8, 747.2 cm⁻¹.

LCMS (m/z): 582.9 (M+1, 85%).

### 3-{(4-Methylbenzyl)-[4-[3-(2-methoxyphenyl)ureido]-6-(naphthalen-2-yloxy)-[1,3,5]triazin-yl]amino}propionic acid (Compound No. 47)

3-{(4-Methylbenzyl)-[4-[3-(2-methoxyphenyl)ureido]-6-(naphthalen-2-yloxy)-[1,3,5]triazin-yl]amino}propionic acid was prepared by using 3-(4-methylbenzyl)aminopropionic acid instead of 3-(3-fluorobenzyl)aminopropionic acid in Step 3, Example 4.

m.p.: 202-205° C.

¹HNMR (DMSO-d₆, 300 MHz): δ 12.2 (1H, bs, CO₂H), 11.19 (s) & 11.07 (s) [1H, NH], 10.16 (1H, s, NH), 7.80-8.05 (5H, m), 7.40-7.5 (3H, m) & 6.85-7.15 (7H, m) [aromatic], 4.45 (s) & 3.90 (s) [2H, NCH₂Ar], 3.76 (bs) & 3.70 (bs) [5H, NCH₂, and OCH₃] and 2.41 (1H, m, CH₂CO₂H) and 2.24 (3H, s, Ar CH₃).

IR (νₘₐₓ, KBr): 1713.0, 1597.6, 1402.9, 1243.3, 748.9 cm⁻¹.

LCMS (m/z): 579.2 (M+1, 100%).

### 3-{Benzo[1,3]dioxo-5-ylmethyl-[4-[3(2-methoxyphenly-ureido]-6-(naphthalene-2-yloxy)-[1,3,5]triazin-2-yl]amino}propionic acid (Compound No. 50)

3-{Benzo[1,3]dioxo-5-ylmenthuyl-[4-[3(2-methoxyphenly-ureido-6-(naphthalene-2-yloxy)-[1,3,5]triazin-2-yl]amino}propionic acid was prepared by using 3-(benzo[1,3]dioxo-5-ylmethyl)aminopropionic acid instead of 3-(3-fluorobenzyl)aminopropionic acid in Step 3, Example 4.

m.p.: 214-222° C.

¹HNMR (TFA-d, 300 MHz): δ 7.50-9.10 (14H, m, aromatic), 7.06 (s) & 7.02 (s) [2H, OCH₂O], 5.98 (s) & 5.66 (s) [2H, NCH₂Ph], 5.16 (s) & 5.04 (s) [5H, NCH₂ and OCH₃] and 3.92 (s) & 3.53 (s) [2H, CH₂ CO₂H].

IR (νₘₐₓ, KBr): 1715.2, 1596.4, 1558.0, 1243.9, 748.9 cm⁻¹.

LCMS (m/z): 609.1 (M+1, 100%).

### 3-{(2-Methoxybenzyl)-[4-[3-(2-methoxyphenyl)ureido]-6-(naphthalen-2-yloxy)-[1,3,5]triazin-2-yl]amino}propionic acid (Compound No. 51)

3-{(2-Methoxybenzyl)-[4-[3-(2-methoxyphenyl)ureido]-6-(naphthalen-2-yloxy)-[1,3,5]triazin-2-yl]amino}propionic acid was prepared by using 3-(2-methoxybenzyl)aminopropionic acid instead of (3-fluorobenzyl)amino propionic in Step 3, Example 4.

m.p.: 185-197° C.

¹HNMR (TFA-d, 300 MHz): δ 7.8-9.10 (15H, m, aromatic), 6.12 (s) & 5.82 (s) [2H, NCH₂ Ar], 5.40 (bs) & 4.87 (bs) [2H, NCH₂], 5.19 (s), 5.06 (s), 5.03 (s) & 4.97 (s) [6H, 2×OCH₃] and 3.98 (s) & 3.50 (s) [2H, CH₂CO₂H].

IR (νₘₐₓ, KBr): 1713.5, 1597.0, 1244.2, 1028.9. 750.3 cm⁻¹.

LCMS (m/z): 595.1 (M+1, 85%).

### 3-{(3,4-Dimethoxybenzyl)-[4-[3-(2-methoxyphenyl)ureido]-6-(naphthalen-2-yloxy)-[1,3,5]triazin-2-yl]amino}propionic acid (Compound No. 52)

3-{(3,4-Dimethoxybenzyl)-[4-[3-(2-methoxyphenyl)ureido]-6-(naphthalen-2-yloxy)-[1,3,5]triazin-2-yl]amino}propionic acid was prepared by using 3-(3,4-dimethoxybenzyl)aminopropionic acid instead of 3-(3-fluorobenzyl)aminopropionic acid in Step 3, Example 4.

m.p.: 190-196° C.

¹HNMR (DMSO-d₆, 300 MHz): δ 11.26 (s) & 11.11 (s) (1H, NH), 10.17 (s) & 10.16 (s) [1H, NH], 7.80-8.10 (5H, m), 7.50 (3H, m), 6.50-7.00 (6H, m) [aromatic], 4.86 (s) & 4.44 (s) [2H, NCH₂ Ar], 3.83 (s), 3.77 (s), 3.71 (s), 3.69 (s) & 3.60 (s) [11H, NCH₂ and OCH₃×3] and 2.40 (m, CH₂ CO₂H).

IR (νₘₐₓ, KBr): 1710.8, 1596.4, 1242.7, 1027.9, 750 cm⁻¹.

LCMS (m/z): 625.1 (M+1, 100%).

### 3-{(3-Methylbenzyl)-[4-[3-(2-methoxyphenyl)ureido]-6-(naphthalen-2-yloxy)-[1,3,5]triazin-2-yl]amino}propionic acid (Compound No. 53)

3-{(3-Methylbenzyl)-[4-[3-(2-methoxyphenyl)ureido]-6-(naphthalen-2-yloxy)-[1,3,5]triazin-2-yl]amino}propionic acid was prepared by using 3-(3-methylbenzyl)aminopropionic acid instead of 3-(3-fluorobenzyl)aminopropionic acid in Step 3, Example 4.

m.p.: 105-125° C.

¹HNMR (TFA-d, 300 MHz): δ 7.80-9.15 (15H, m, aromatic), 6.20 (s), 6.08 (s) & 5.76 (s) [2H, NCH₂ Ar], 5.42 (bs), 4.99 (bs) & 4.78 (bs) [NCH₂], 5.20 (s), 5.08 (s), 5.05 (s) [3H, OCH₃], 3.95 (bs), 3.53 (bs) & 3.48 (bs), [2H, CH₂CO₂H] and 3.43 (s), 3.32 (s) & 3.21 (s) [3H, ArCH₃].

IR (νₘₐₓ, KBr): 1709.6, 1596.0, 1245.6, 749.3 cm⁻¹.

LCMS (m/z): 579.2 (M+1, 100%).

### 3-{(2-Fluorobenzyl)-[4-[3-(2-methoxyphenyl)ureido-6-(naphthalen-2-yloxy)-[1,3,5]triazin-2-yl]amino}propionic acid (Compound No. 54)

3-{(2-Fluorobenzyl)-[4-[3-(2-methoxyphenyl)ureido-6-(naphthalen-2-yloxy)-[1,3,5]triazin-2-yl]amino}propionic acid was prepared by using 3-(2-fluorobenzyl)aminopropionic acid instead of 3-(3-fluorobenzyl)aminopropionic acid in Step 3, Example 4.

m.p.: 218-230° C.

¹HNMR (TFA-d, 300 MHz): δ 7.6-9.15 (15H, m, aromatic), 6.18 & 5.82 (s) [2H, NCH₂Ar], 5.28 (bs) & 4.78 (m), [2H, NCH₂], 5.08 (s) & 5.07 (s) [3H, OCH₃] and 3.99 (bs) & 3.51 (m) [2H, CH₂CO₂H].

IR (νₘₐₓ, KBr): 1716.5, 1594.9, 1556.0, 1361.6, 1244.0, 750.6 cm⁻¹.

LCMS (m/z): 583.1 (M+1, 100%).

### 3-{(2-Chlorobenzyl)-[4-[3-(2-methoxyphenyl)ureido-6-(naphthalen-2-yloxy)-[1,3,5]triazin-2-yl]amino]propionic acid (Compound No. 55)

3-{(2-Chlorobenzyl)-[4-[3-(2-methoxyphenyl)ureido-6-(naphthalen-2-yloxy)-[1,3,5]triazin-2-yl]amino]propionic acid was prepared by using 3-(2-chlorobenzyl)aminopropionic acid instead of 3-(3-fluorobenzyl)aminopropionic acid in Step 3, Example 4.

m.p.: 190-215° C.

¹HNMR (TFA-d, 300 MHz): δ 8.15-9.10 (17H, m, aromatic and 2NH), 6.23 (s) & 5.88 (s) [24, NCH₂Ar], 5.22 (bs) & 4.76 (bs) [2H, NCH₂], 5.06 (s) & 5.02 (s) [3H, OCH₃] and 3.93 (bs) & 3.48 (bs) [CH₂CO₂H].

IR (νₘₐₓ, KBr): 1717.5, 1598.8, 1537.4, 1243.3, 749.4 cm⁻¹.

LCMS (m/z): 599.0 (M+1, 100%).

### 3-{(2,4-Dichlorobenzyl)-[4-[3-(2-methoxyphenyl)ureido-6-(naphthalen-2-yloxy)-[1,3,5]triazin-2-yl]amino}propionic acid (Compound No. 56)

3-{(2,4-Dichlorobenzyl)-[4-[3-(2-methoxyphenyl)ureido-6-(naphthalen-2-yloxy)-[1,3,5]triazin-2-yl]amino}propionic acid was prepared using 3-(2,4-dichlorobenzyl)aminopropionic acid instead of 3-(3-fluorobenzyl)aminopropionic acid in Step 3, Example 4.

m.p.: 225-230° C.

¹HNMR (TFA-d, 300 MHz): δ 7.85-9.05 (16H, m, aromatic and 2NH), 6.22 (s) & 5.81 (s) [2H, NCH₂ Ar], 5.27 (bs) & 4.78 (bs) [2H, NCH₂], 5.09 (s) & 5.06 (s) [3H, OCH₃] and 4.01 (bs) & 3.54 (m) [2H, CH₂CO₂H].

IR (νₘₐₓ, KBr): 1710.4, 1597.9, 1368.3, 750.4 cm⁻¹.

LCMS (m/z): 633.2 (M+1, 65%).

### 3-{(3,4-Dichlorobenzyl)-[4-[3-(2-methoxyphenyl)ureido-6-(naphthalen-2-yloxy)-[1,3,5]triazin-2-yl]amino}propionic acid (Compound No. 57)

3-{(3,4-Dichlorobenzyl)-[4-[3-(2-methoxyphenyl)ureido-6-(naphthalen-2-yloxy)-[1,3,5]triazin-2-yl]amino}propionic acid was prepared by using 3-(3,4-dichlorobenzyl)aminopropionic instead of 3-(3-fluorobenzyl)aminopropionic acid in Step 3, Example 4.

m.p.: 194-206° C.

¹HNMR (TFA-d, 300 MHz): δ 8.00-8.70 (19H, m, aromatic and 2NH), 5.99 (s) & 5.89 (s) [2H, NCH₂Ar], 5.14 (bs), 5.03 (s) & 5.01 (s) [5H, NCH₂ and OCH₃], 3.74 (bs) & 3.51 (bs) [2H, CH₂CO₂H], 3.43 (s) & 3.40 (s) [3H, Ar—CH₃].

IR (νₘₐₓ, KBr): 1709.4, 1599.7, 1534.3, 1249.8, 746.9 cm⁻¹.

LCMS (m/z): 604.1 (M+1, 90%).

### 3-{(1H-Indol-3-yl)-2S-([4-(2-methoxyphenyl)ureido]-6-(naphthalen-2-yloxy)-[1,3,5]triazin-2-yl)amino}propionic acid (Compound No. 60)

3-{(1H-Indol-3-yl)-2S-([4-(2-methoxyphenyl)ureido]-6-(naphthalen-2-yloxy)-[1,3,5]triazin-2-yl)amino}propionic acid was prepared by using 2S-amino-3-(1H-indol-3-yl)propionic acid (L-tryptophan) instead of 3-(3-fluorobenzyl)aminopropionic acid in Step 3, Example 4.

m.p.: 147-152° C.

¹HNMR (DMSO-d₆, 300 MHz): δ 11.41 (s) & 11.19 (s) [1H, CO₂H], 10.83 (s) & 10.78 (s) [1H, NH], 10.03 (1H, s, NH), 7.90-8.00 (5H, m), 7.78 (1H, bs), 7.25-7.55 (6H, m) & 6.85-7.10 (6H, m), [aromatic and 2NH], 4.81 (t) & 4.47 (bs) [1H, NCHCO₂H], 3.74 (s) & 3.64 (s) [3H, OCH₃] and 3.15 (m, CH₂ Ar).

LCMS (m/z): 590.2 (M+1, 100%).

### 2-{4-[3-(2-Methoxyphenyl)ureido]-6-(naphthalen-2-yloxy)-[1,3,5]triazin-2-yl} 1,2,3,4-tetrahydro-isoquinoline-3-carboxylic acid (Compound No. 61)

2-{4-[3-(2-Methoxyphenyl)ureido]-6-(naphthalen-2-yloxy)-[1,3,5]triazin-2-yl} 1,2,3,4-tetrahydro-isoquinoline-3-carboxylic acid was prepared by using 1,2,3,4-tetrahydro-isoquinoline-3-carboxylic acid instead of 3-(3-fluorophenyl)aminopropionic acid in Step 3, Example 4.

m.p.: 230-235° C.

¹HNMR (DMSO-d₆, 300 MHz): δ 11.14 (s) & 10.90 (s) [1H, CO₂H], 10.20 (s) & 10.10 (s) [1H, NH], 8.08 (d, J=9 Hz), 7.75-8.00 (5H, m), 7.52 (d, J=9 Hz), 7.44 (bs) & 7.38 (bs) [3H], 7.16 (bs) & 7.01 (m) [7H] [aromatic, 1NH], 5.61 (s) & 5.16 (s), [1H, NCHCO₂H], 5.06 (d) & 4.75 (d) [1H] & 4.82 (1H, t) [NCH₂Ar], 3.81 (s) & 3.77 (s) [3, OCH₃] and 3.17 (2H, bs, CH₂ Ar).

IR (νₘₐₓ, KBr): 1709.5, 1594.8, 1528.2, 1243.0, 748.7 cm⁻¹.

LCMS (m/z): 563.1 (M+1, 100%).

### 2-{(4-Fluorobenzyl)-[4-[3-(2-methoxyphenyl)ureido-6-(naphthalen-2-yloxy)-[1,3,5]triazin-2-yl]amino}-acetic acid (Compound No. 62)

2-{(4-Fluorobenzyl)-[4-[3-(2-methoxyphenyl)ureido-6-(naphthalen-2-yloxy)-[1,3,5]triazin-2-yl]amino}-acetic acid was prepared by using 2-(4-fluorobenzyl)aminoacetic acid instead of 3-(3-fluorobenzyl)aminopropionic acid in Step 3, Example 4.

m.p.: 196-200° C.

¹HNMR (TFA-d, 300 MHz): δ 7.90.9.20 (15H, m, aromatic), 6.31 (s), 6.21 (s), 5.83 (s), 5.75 (s), 5.73 (s), 5.57 (s), 5.44 (s), 5.31 (s), 5.27 (s), 5.15 (s) & 5.13 (s) [7H, NCH₂Ar, NCH₂CO₂H and OCH₃].

LCMS (m/z): 569.0 (M+1, 100%).

### 2-{(4-Methoxybenzyl)-[4-[3-(2-methoxyphenyl)ureido-6-(naphthalen-2-yloxy)-[1,3,5]triazin-2-yl]amino}acetic acid (Compound No. 63)

2-{(4-Methoxybenzyl)-[4-[3-(2-methoxyphenyl)ureido-6-(naphthalen-2-yloxy)-[1,3,5]triazin-2-yl]amino}acetic acid was prepared by using 2-(4-methoxybenzyl)aminoacetic acid instead of 3-(3-fluorobenzyl)amino propionic acid in Step 3, Example 4.

m.p.: 214-217° C.

¹HNMR (DMSO-d₆, 300 MHz): δ 11.10 (s) & 11.02 (s) [1H, CO₂H], 10.21 (s) & 10.14 (s) [1H, NH], 7.90-8.02 (4H, m), 7.86 (1H, d, J=18 Hz), 7.50 (3H, m), 7.25 (1H, d, J=6 Hz), 6.55-7.00 (5H, m) and 6.57 (1H, d, J=6 Hz), 4.83 (s) & 3.91 (s), [2H, NCH₂Ar], 4.35 (s) & 4.30 (s), [2H, NCH₂CO₂H] and 3.82 (s), 3.75 (s), 3.69 (s) & 3.65 (s) [6H, 2×OCH₃].

IR (νₘₐₓ, KBr): 1750.7, 1706.2, 1594.2, 1536.5, 1246.4, 745.3 cm⁻¹.

LCMS (m/z): 581.2 (M+1, 100%).

### 2-{(4-Methylbenzyl)-[4-[3-(2-methoxyphenyl)ureido-6-(naphthalen-2-yloxy)-[1,3,5]triazin-2-yl]amino}acetic acid (Compound No. 64)

2-{(4-Methylbenzyl)-[4-[3-(2-methoxyphenyl)ureido-6-(naphthalen-2-yloxy)-[1,3,5]triazin-2-yl]amino} acetic acid was prepared by using 2-(4-methylbenzyl)aminoacetic acid instead of 3-(3-fluorobenzyl)aminopropionic acid in Step 3, Example 4.

m.p.: 205-210° C.

¹HNMR (TFA-d, 300 MHz): δ 7.6-8.80 (15H, m, aromatic), 5.81 (s) & 4.94 (s), [2H, NCH₂Ar], 5.46 (s) & 5.36 (s) [2H, NCH₂CO₂H], 4.90 (s) & 4.70 (s) [3H, OCH₃] and 3.15 (s) & 3.09 (s), [3H, ArCH₃].

IR (νₘₐₓ, KBr): 1702.1, 1594.2, 1244.1, 747.1 cm⁻¹.

LCMS (m/z): 565.1 (M+1, 100%).

### 3-{(3,4-Difluorobenzyl)-[4-[3-(2-methoxyphenyl)ureido-6-naphthalen-2-yloxy-[1,3,5]triazin-2-yl]amino}propanoic acid (Compound No. 65)

3-{(3,4-Difluorobenzyl)-[4-[3-(2-methoxyphenyl)ureido-6-naphthalen-2-yloxy)-[1,3,5]triazin-2-yl]amino}propanoic acid was prepared by using 3-(3,4-difluorobenzyl)aminopropionic acid instead of 3-(3-fluorobenzyl)aminopropionic acid in Step 3, Example 4.

m.p.: 194-196° C.

¹HNMR (TFA-d, 300 MHz): δ 8.50-9.50 (14H, m, aromatic), 6.15 (s) & 5.94 (s) [2H, NCH₂Ar], 5.64 (bs) & 5.43 (bs) [2H, NCH₂], 5.53 (s) & 5.52 (s) [3H, OCH₃] and 5.22 (t) & 4.46 (t) [2H, CH₂CO₂H].

IR (νₘₐₓ, KBr): 1701.4, 1596.3, 1553.5, 1243.5, 746.4 cm⁻¹.

### 3-{4-[3-(2-Methoxyphenyl)ureido]-6-(naphthalen-2-yloxy)-[1,3,5]triazin-2-yl]-thiophen-2-yl-methylamino}propionic acid (Compound No. 66)

3-{4-[3-(2-Methoxyphenyl)ureido]-6-(naphthalen-2-yloxy)-[1,3,5]triazin-2-yl]-thiophen-2-yl-methylamino}propionic acid was prepared by using 3-{(thiophen-2-yl)methylamino}propionic acid instead of 3-(3-fluorobenzyl)aminopropionic acid in Step 3, Example 4.

m.p.: 204-208° C.

¹HNMR (TFA-d, 300 MHz): δ 7.00-8.50 (14H, m, aromatic), 5.53 (s) & 5.25 (s) [2H, NCH₂Ar], 4.48 (t) & 4.12 (t) [2H, NCH₂], 4.37 (s) & 4.28 (s) [3H, OCH₃] and 3.28 (t) & 2.93 (t) [CH₂CO₂H].

IR (νₘₐₓ, KBr): 1710.8, 1596.4, 1536.5, 1241.2, 748.7 cm⁻¹.

LCMS (m/z): 571.1 (M+1, 100%).

### 3-{(4-Trifluoromethylbenzyl)-[4-[3-(2-methoxyphenyl-ureido-6-(naphthalen-2-yloxy}-[1,3,5]triazin-2-yl]amino}propanoic acid (Compound No. 67)

3-{(4-Trifluoromethylbenzyl)-[4-[3-(2-methoxyphenyl)ureido-6-(naphthalen-2-yloxy}-[1,3,5]triazin-2-yl]amino}propanoic acid was prepared by using 3-(4-trifluoromethylbenzyl)aminopropionic acid instead of 3-(3-fluorobenzyl)aminopropionic acid in Step 3, Example 4.

m.p.: 198-200° C.

¹HNMR (TFA-d, 300 MHz): δ 9.10-9.50 (m), 8.84-8.95 (m), 8.66 (m) & 8.44 (d, J=9 Hz) [17H, aromatic and 2NH], 6.63 (s) & 6.24 (s), [2H, NCH₂Ar], 5.74 (t) 5.25 (t, J=9 Hz) [2H, NCH₂] and 5.53 (s), 5.50 (s), 5.47 (s) & 5.43 (s) [3H, OCH₃].

IR (νₘₐₓ, KBr): 1717.5, 1598.0, 1541.0, 1327.0, 1166.3, 747.0 cm⁻¹.

LCMS (m/z): 633.4 (M+1, 100%).

### 3-{[2-(1H-Indol-3-yl)ethyl]-[4-[3-(2-methoxyphenyl)ureido]-6-(naphthalen-2-yloxy)]-[1,3,5]triazin-2-yl]amino}propanoic acid (Compound No. 68)

3-{[2-(1H-Indol-3-yl)ethyl]-[4-[3-(2-methoxyphenyl)ureido]-6-(naphthalen-2-yloxy)]-[1,3,5]triazin-2-yl]amino}propanoic acid was prepared by using 3-[(2-(1H-indol-3-yl)ethyl]aminopropionic acid instead of 3-(3-fluorobenzyl)aminopropionic acid in Step 3, Example 4.

m.p.: 114-120° C.

¹HNMR (DMSO-d₆, 300 MHz): δ 11.24 (s) & 11.18 (s) [1H, NH], 10.75 (s) & 10.66 (s) [1H, NH], 10.12 (s) & 10.10 (s) [1H, NH], 7.80-8.10 (m), 7.51 (m), 7.27 (t, J=9 Hz), 6.9-7.05 (m) & 6.64 (m) [16H, aromatic], 3.91 (m), 3.87 (s), 3.85 (s), 3.78 (s) & 3.64 (bs) [NCH₂×2 and OCH₂], 3.35-3.50 (2H, m, ArCH₂) and 2.93 (t) & 2.77 (t) [2H, CH₂CO₂H].

IR (νₘₐₓ, KBr): 1705.2, 1596.6, 1559.9, 1244.1, 746.6 cm⁻¹.

LCMS (m/z): 618.2 (M+1, 100%).

### 1-[4-[2-(4-Hydroxyphenyl)ethylamino]-6-(naphthalen-2-yloxy}-[1,3,5]triazin-2-yl]-3-(2-methoxyphenyl)urea (Compound No. 69)

1-[4-[2-(4-Hydroxyphenyl)ethylamino]-6-(naphthalen-2-yloxy}-[1,3,5]triazin-2-yl]-3-(2-methoxyphenyl)urea was prepared by using 2-(4-hydroxyphenyl)ethylamine in place of 3-(3-fluorophenyl)aminopropionic acid in Step 3, Example 4.

m.p.: 238° C.

¹HNMR (DMSO-d₆, 300 MHz): δ 11.62 (s) & 11.27 (s) [1H, NH], 10.09 (s) & 9.999 (s) [1H, NH], 9.15 (s) & 9.08 (s) [1H, NH], 7.79-8.10 (6H, m), 7.50-7.57 (3H, m), 7.02 (bs), 6.95 (d, J=8.1 Hz), 6.88 (d, J=8.4 Hz) [4H], 6.53 (1H, d, J=8.4 Hz) & 6.48 (dd), [aromatic], 3.82 (s) & 3.62 (s) [3H, OCH₃], 3.48 (m) & 3.06 (t) [2H, NCH₂] and 2.65 (t) & 2.43 (t) [2H, CH₂ Ar].

IR (νₘₐₓ, KBr): 1702.5, 1600.1, 1343.9, 1243.5, 743.8 cm⁻¹.

LCMS (m/z): 523.3 (M+1, 100%).

### 1-[4-[2-(1H-Indol-3-yl)ethylamino]-6-(naphthalen-2-yloxy)-[1,3,5]triazin-2-yl]-3-(2-methoxyphenyl)urea (Compound No. 70)

1-[4-[2-(1H-Indol-3-yl)ethylamino]-6-(naphthalen-2-yloxy)-[1,3,5]triazin-2-yl]-3-(2-methoxyphenyl)urea was prepared by using 2-(1H-indol-2-yl)ethylamine in place of 3-(3-fluorobezyl)aminopropionic acid in Step 3, Example 4.

m.p.: 243-248° C.

¹HNMR (DMSO-d₆, 300 MHz): δ 11.55 (s) & 11.34 (s) [1H, NH], 10.75 (s) & 10.72 (s) [1H, NH], 10.07 (s) & 9.98 (s) [1H, NH], 7.79-8.10 (m), 7.51 (m), 7.43 (d, J=8.4 Hz), 7.28 (t), 6.65-7.05 (m) [16H aromatic], 3.77 (bs) & 3.63 (bs) & [OCH₃] and 2.89 (t, J=7.2 Hz) and 2.79 (t, J=8.1 Hz) [2H, CH₂Ar].

IR (νₘₐₓ, KBr): 1696.5, 1599.7, 1546.9, 1245.6, 741.6 cm⁻¹.

LCMS (m/z): 546.2 (M+1, 100%).

### 1-(2-Methoxyphenyl)-3-[(4-(3-methylbenzyl)amino)-6-(nalphthalen-2-yloxy)-[1,3,5]triazin-2-yl]urea (Compound No. 71)

1-(2-Methoxyphenyl)-3-[(4-(3-methylbenzyl)amino)-6-(naphthalen-2-yloxy)-[1,3,5]triazin-2-yl]urea was prepared by using 3-methylbenzylamine instead of 3-(3-fluorophenyl)aminopropionic acid in Step 3, Example 4.

m.p.: 245-247° C.

¹HNMR (TFA-d, CDCl₃, 300 MHz): δ 7.00-8.37 (15H, m, aromatic), 5.12 (s) & 4.73 (AB_{q}) [2H, NCH₂Ar], 4.25 (s) & 3.93 (bs) [3H, OCH₃], 2.63 (s) & 2.55 (s) [3H, Ar CH₃].

IR (νₘₐₓ, KBr): 1701.5, 1603.5, 1246.1, 744.5 cm⁻¹.

LCMS (m/z): 507.2 (M+1, 100%).

### 1-[(4-(3,4-Dichlorobenzyl)amino)-6-(naphthalen-2-yloxy)-[1,3,5]triazin-2-yl]-3-(2-methoxyphenyl)urea (Compound No. 72)

1-[(4-(3,4-Dichlorobenzyl)amino)-6-(naphthalen-2-yloxy)-[1,3,5]triazin-2-yl]-3-(2-methoxyphenyl)urea was prepared by using 3,4-dichlorobenzylamine instead of 3-(3-fluorobenzyl)aminopropionic acid in Step 3, Example 4.

m.p.: 265-268° C.

¹HNMR (TFA-d, CDCl₃, 300 MHz): δ 7.05-8.47 (14H, m, aromatic), 5.17 (s) & 4.75 (AB_{q}, J=8.1 Hz) [2H, NCH₂Ar], 4.33 (s) & 3.93 (bs) [3H, OCH₃].

LCMS (m/z): 561.1 (M+1, 100%)

### 4-[{4-{3-(2-Methoxyphenyl)ureido}-6-(naphthalen-2-yloxy)-[1,3,5]triazin-2-yl}amino]phenylacetic acid (Compound No. 79)

4-[{4-{3-(2-Methoxyphenyl)ureido}-6-(naphthalen-2-yloxy)-[1,3,5]triazin-2-yl}amino]phenylacetic acid was prepared by using 4-aminophenylacetic acid in place of 3-(3-fluorophenyl)amino propionic acid in Step 3, Example 4.

m.p.: 232-240° C.

¹HNMR (TFA-d, CDCl₃, 300 MHz): δ 8.2-9.21 (15H, m, aromatic) and 5.15 (s), 5.10 (s), 5.07 (s), 4.89 (s) [5H, OCH₃ and CH₂CO₂H].

IR (νₘₐₓ, KBr): 1704.9, 1665.1, 1548.6, 1347.2, 1164.3 cm⁻¹.

LCMS (m/z): 537.0 (M+1, 100%).

### 3-{[4-[3-(2-Methoxyphenyl)ureido]-6-(naphthalen-2-yloxy)-[1,3,5]triazin-2-yl]-methylamino}-propionic acid (Compound No. 104)

3-{[4-[3-(2-Methoxyphenyl)ureido]-6-(naphthalen-2-yloxy)-[1,3,5]triazin-2-yl]-methylamino}-propionic acid was prepared by using 3-(methylamino)propionic acid instead of 3-(3-fluorobenzyl)amino propionic acid in Step 3, Example 4.

m.p.: 178-180° C.

¹HNMR (DMSO-d₆, 300 MHz): δ 11.04 (s) & 11.01 (s), [1H, NH], 10.17 (s) & 10.11 (s) [1H, NH], 7.75-8.10 (4H, m), 7.19-7.55 (4H, m), 6.9-7.05 (3H, m) & [aromatic], 4.43 (s) & 4.11 (s) [2H, NCH₂], 3.81 (s) & 3.78 (s) [3H, OCH₃], 3.17 (s) & 2.91 (s) [3H, N—CH₃]

IR (νₘₐₓ, KBr): 1717.1, 1597.4, 1242.5, 748.5 cm⁻¹.

LCMS (m/z): 475.3 (M+1, 100%).

### 3-{(3-Chlorobenzyl)-[4-[3-(2-methoxyphenyl)ureido]-6-(naphthalen-2-yloxy)-[1,3,5]triazin-2-yl]amino}propionic acid (Compound No. 106)

3-{(3-Chlorobenzyl)-[4-[3-(2-methoxyphenyl)ureido]-6-(naphthalen-2-yloxy)-[1,3,5]triazin-2-yl]amino}propionic acid was prepared by using 3-(3-chlorobenzyl)aminopropionic acid instead of 3-(3-fluorobenzyl)aminopropionic acid in Step 3, Example 4.

m.p.: 215-218° C.

¹HNMR (TFA-d, 300 MHz): δ 7.35-8.58 (15H, m, aromatic), 5.58 (s) & 5.24 (s) [2H, NCH₂Ar], 4.73 (t) & 4.29 (t) [2H, NCH₂], 4.57 (s) & 4.54 (s) [3H, OCHz] and 3.50 (s) & 3.08 (s) [2H, CH₂CO₂H].

IR (νₘₐₓ, KBr): 1715.7, 1537.0, 1243.0, 747.7 cm⁻¹.

LCMS (m/z): 599.2 (M+1, 20%).

### 3-{[4-[3-(2-Methoxyphenyl)ureido]-6-(naphthalen-2-yloxy)-[1,3,5]triazin-2-yl]-(2-trifluoromethylbenzyl)amino}propionic acid (Compound No. 107)

3-{[4-[3-(2-Methoxyphenyl)ureido]-6-(naphthalen-2-yloxy)-[1,3,5]triazin-2-yl]-(2-trifluoromethylbenzyl)amino}propionic acid was prepared by using 3-(2-trifluoromethylbenzyl)aminopropionic acid instead of 3-(3-fluorobenzyl)aminopropionic acid in Step 3, Example 4.

m.p.: 232-233° C.

¹HNMR (TFA-d, 300 MHz): δ 8.00-8.70 (11H, m), 7.74 (4H, m) [aromatic], 5.87 (s) & 5.54 (s) [2H, NCH₂Ar], 4.75 (t) & 4.30 (t, J=6 Hz) [2, NCH₂], 4.60 (s) & 4.54 (s) [3H, OCH₃], 3.52 (t) & 3.13 (t, J=9 Hz) (2H, CH₂CO₂H).

LCMS (m/z): 633.5 (M+1, 8.5%).

### 3-{[4-[3-(2-Methoxyphenyl)ureido]-6-(naphthalen-2-yloxy)-[1,3,5]triazin-2-yl]-(3-trifluoromethylbenzyl)aminopropionic acid (Compound No. 108)

3-{[4-[3-(2-Methoxyphenyl)ureido]-6-(naphthalen-2-yloxy)-[1,3,5]triazin-2-yl]-(3-trifluoromethylbenzyl)aminopropionic acid was prepared by using 3-(3-trifluoromethylbenzyl)aminopropionic acid instead of 3-(3-fluorobenzyl)aminopropionic acid in Step 3, Example 4.

m.p.: 185-188° C.

¹HNMR (TFA-d, 300 MHz): δ 8.00-8.60 (9H, m), 7.50-8.00 (6H, m) [aromatic] 5.67 (s) & 5.33 (s) [2H, NCH₂Ar], 4.73 (t) & 4.29 (t) [2H, NCH₂], 4.57 (s) & 4.54 (s) [3H, OCH₃] and 3.50 (t) [2H, CH₂CO₂H].

IR (νₘₐₓ, KBr): 1716.9, 1596.9, 1123.3, 748.4 cm⁻¹.

LCMS (m/z): 633.5 (M+1, 75%).

### 3-{(2,4-Bis-trifluoromethylbenzyl)-[4-[3-(2-methoxyphenyl)ureido]-6-(naphthalen-2-yloxy)-[1,3,5]triazin-2-yl]amino}propionic acid (Compound No. 109)

3-{(2,4-Bis-trifluoromethylbenzyl)-[4-[3-(2-methoxyphenyl)ureido]-6-(naphthalen-2-yloxy)-[1,3,5]triazin-2-yl]amino}propionic acid was prepared by using 3-(2,4-bis trifluoromethylbenzyl)aminopropionic acid instead of 3-(3-fluorobenzyl)aminopropionic acid in Step 3, Example 4.

m.p.: 192° C.

¹HNMR (TFA-d, 300 MHz): δ 7.6-8.6 (14H, m, aromatic), 5.76 (s) & 5.42 (s) [2H, NCH₂Ar], 4.72 (t) & 4.31 (t) [2H, NCH₂], 4.58 (s) & 4.55 (s) [3H, OCH₃], 3.53 (t) & 3.16 (t, J=6 Hz) [2H, CH₂CO₂H].

IR (νₘₐₓ, KBr): 1728.9, 1560.0, 1280.4, 747.3 cm⁻¹.

LCMS (m/z): 701.3 (M+1, 65%).

### 3-{(Biphenyl-4-ylmethyl)-[4-[3-(2-methoxyphenyl)ureido]-6-naphthalen-2-yloxy-[1,3,5]triazin-2-yl]amino}propionic acid (Compound No. 110)

3-{(Biphenyl-4-ylmethyl)-[4-[3-(2-methoxyphenyl)ureido]-6-naphthalen-2-yloxy)-[1,3,5]triazin-2-yl]amino}propionic acid was prepared by using 3-(biphenyl-4-ylmethyl)aminopropionic acid instead of 3-(3-fluorobenzyl)aminopropionic acid in Step 3, Example 4.

m.p.: 202-203° C.

¹HNMR (TFA-d, 300 MHz): δ 7.50-8.60 (20H, m, aromatic), 5.64 (s) & 5.30 (s), [2H, NCH₂Ar], 4.79 (t) & 4.32 (t) [2H, NCH₂], 4.57 (s) & 4.53 (s) [3H, OCH₃] and 3.52 (t) & 3.08 (t) [2H, CH₂CO₂H].

IR (νₘₐₓ, KBr): 1710.6, 1595.8, 1245.7, 748.2 cm⁻¹.

LCMS (m/z): 641.4 (M+1, 100%).

### 3-{[4-[3-(2-Methoxyphenyl)ureido]-6-(naphthalen-2-yloxy)-[1,3,5]triazin-2-yl]-(naphthalen-1-ylmethyl)amino}-propionic acid (Compound No. 111)

3-{[4-[3-(2-Methoxyphenyl)ureido]-6-(naphthalen-2-yloxy)-[1,3,5]triazin-2-yl]-(naphthalen-1-ylmethyl)amino}-propionic acid was prepared by using 3-(napthalen-1-ylmethyl)aminopropionic acid instead of 3-(2-fluorobenzyl)aminopropionic acid in Step 3, Example 4.

m.p.: 230-233° C.

¹HNMR (TFA-d, 300 MHz): δ 7.60-8.60 (18H, m, aromatic), 6.08 (s) & 5.77 (s) [2H, NCH₂Ar], 4.77 (t) & 4.30 (t) [2H, NCH₂], 4.57 (s) & 4.49 (s) [3H, OCH₃] and 3.33 (t) & 2.83 (t) [2H, CH₂CO₂H].

IR (νₘₐₓ, KBr): 1717.5, 1558.2, 1244.7, 748.2 cm⁻¹.

LCMS (m/z): 615.3 (M+1, 70%).

### Example 5

### Preparation of 2-[-4-[3-(2-Methoxyphenyl)ureido]-6-(napthalen-2-yloxy)-[1,3,5]triazin-2-yl] malonic acid di tert butyl ester (Compound No. 5)

### Step 1: Preparation of [2-amino-6-(naphthalen-2-yloxy)-[1,3,5]triazin-2-yl] malonic acid di tert butyl ester

To a suspension of sodium hydride (352 mg, 50% dispersion in oil, 7.34 mmol) in dry THF (40 ml) at 0° C. was added di-tert-butyl malonate (1.59 g, 7.34 mmol) slowly and stirred for 30 min. Maintaining the same temperature, 4-chloro-6-(naphthalen-2-yloxy)-[1,3,5]triazin-2-ylamine (prepared as in Example 4) (1 gm, 3.67 mmol) was added and stirred for 30 min at 0° C. and then at room temperature overnight. It was poured over ice water and extracted with ethyl acetate (3×50 ml). The combined extracts were washed with brine and dried (Na₂SO₄). Evaporation of the solvent and purification of the residue over a silica gel column using 10-40% EtOAc. Hexane afforded the title compound (850 mg, 53%) as a white solid.

1HNMR (300 MHz, CDCl₃): δ 7.75-7.87 (3H, m), 7.59 (1H, s), 7.45-7.50 (2H, m), and 7.25-7.32 (1H, m) [aromatic], 5.73 (bs) & 5.57 (bs) [H, NH₂], 4.53 (1H, s, Ch) and 1.42 (18H, s, CH₃×6).

IR (νₘₐₓ, KBr): 1746.2, 1644.3, 1374.5, 1137.6, 749.4 cm⁻¹

LCMS (m/z): 453.2 (M+1, 100%).

### Step 2: Preparation of 2-[-4-[3-(2-methoxyphenyl)ureido]-6-(napthalen-2-yloxy)-[1,3,5]triazin-2-yl]malonic acid di tert butyl ester

To a solution of the intermediate (160 mg, 0.35 mmol) obtained from Step 1 Example 5 above in dry THF (2 ml) maintained at 0° C., was added sodium hydride (17 mg, 50% dispersion in oil, 0.35 mmol) followed by 2-methoxyphenyl isocyanate (52 mg, 0.35 mmol) and stirred at rt for 3 hours. The mixture was poured over ice water and extracted with ethyl acetate (3×20 ml). The combined extracts were washed with brine and dried (Na₂SO₄) and concentrated. The residue was purified over a silica gel column using 15% ethyl acetate-hexane as eluent to obtain the title compound (140 mg, 67%) as a white solid.

1HNMR (DMSO-d₆, 300 MHz): δ 11.20 (1H, s) and 10.91 (1H, s) [NH×2], 7.84-8.15 (5H, m), 7.52-7.56 (3H, m) and 7.04-7.07 (3H, m) [aromatic], 4.67 (1H, s, CH), 3.86 (3H, s, OCH₃) and 1.24 (18H, s, CH₃×6].

IR (νₘₐₓ, KBr): 1733.5, 1541.8, 1246.3, 1160.8, 748.8 cm⁻¹.

LCMS (m/z): 602.1 (M+1, 100%).

### Example 6

### Preparation of 1-[4-Methoxy-6-(naphthalen-2-yloxy)-[1,3,5]triazin-2-yl]-3-(2-methoxyphenyl)urea (Compound No. 6)

To the chlorotriazine (211 mg, 0.5 mmol) (prepared as in Step 2, Example 4) in THF (9 ml) and methanol (1 ml) was added sodium methoxide (41 mg, 0.75 mmol). The reaction mixture was refluxed for 3 h and poured over cold water (25 ml). The mixture was extracted with ethyl acetate (2×25 ml) and washed with water and brine and dried over anhydrous Na₂SO₄. Evaporation of the solution followed by purification of the residue over silica gel column using 40% EtOAc-hexane as eluent furnished the title compound (110 mg, 53%) as a solid.

1HNMR (CDCl_{3,} 300 MHz): δ 11.26 (1H, s, NH), 8.19 (1H, bs), 7.80-7.89 (3H, m), 7.65 (1H, s), 7.51 (2H, t, J=2.6 Hz), 7.34 and 7.32 (2H, dd, J=9 and 4.8 Hz) and 6.86-7.26 (3H, m) [aromatic] and 3.99 (3H, s) & 3.85 (3H, s) [2×OCH₃].

IR (νₘₐₓ, KBr): 1714.7, 1581.6, 1342.9, 737.3 cm⁻¹.

LCMS (m/z): 418.1 (M+1, 100%).

### Example 7

### Synthesis of 3-[{4-[[Benzo[1,3]dioxo-5-ylmethyl)amino]-6-[3-(2-methoxy phenyl)ureido]-[1,3,5]triazin-2-yl}-(3,4-dichlorobenzyl)amino]propionic acid (Compound No. 97)

### Step 1: Preparation of 3-[(4-amino-6-chloro-[1,3,5]triazin-2-yl)-(3,4-dichloro benzyl)amino]propionic acid

2-amino-4,6-dichloro-1,3,5 triazine (2 g, 12 mmol) and K₂CO₃ (1.68 g, 12 mmol) were added to a solution of the lithium salt of 3-aminopropionic acid derivative (obtained by the LiOH.H₂O (512 mg, 12.1 mmol) hydrolysis of ethyl 3-(3,4-dichlorobenzyl) aminopropionate (3.3 g, 12.1 mmol) in acetone (50 ml) and stirred at room temperature overnight. It was poured into cold water, acidified with HCl and the precipitated solid filtered and dried under vacuum to obtain the title compound (4.3 g, 95%).

m.p.: 105-110° C.

1HNMR (TFA-d, 300 MHz): δ 8.71 (1H, d, J=6 Hz), 8.66 (1H, s) & 8.42 (1H, d, J=9 Hz) [aromatic], 6.28 (s) & 6.23 (s) [2H, NCH₂Ar], 5.33 (t, J=6 Hz) & 5.26 (t, J=6 Hz), [2H, NCH₂] and 4.15 (2H, t, J=6 Hz, CH₂CO₂H).

IR (νₘₐₓ, KBr): 1709.9, 1574.3, 1522.8, 1322.9, 804.6 cm⁻¹.

LCMS (m/z): 375.9 (M+1, 82%), 377.9 (M+3, 100%).

### Step 2: Preparation of 3-[{4-(2-methoxyphenyl)ureido)-6-chloro-[1,3,5]triazin-2-yl}-(3,4-dichlorobenzyl)amino]propionic acid (Compound No. 59)

Following the general protocol, reaction of the aminotriazine (4 g, 10.6 mmol) obtained from Step 1, Example 7, above, with sodium hydride (1.02 g, 50% dispersion in oil, 21.3 mmol) and 2-methoxyphenyl isocyanate (1.58 g, 10.6 mmol) afforded after purification the title compound (3.7 g, 66%) as a white solid.

m.p.: 210-212° C.

1HNMR (TFA-d, 300 MHz): δ 8.3-9.00 (7H, m, aromatic), 5.42 (bs), 5.19 (s) & 5.18 (s) [5H, NCH₂ and OCH₃] and 4.16 (2H, bs, CH₂CO₂H).

IR (νₘₐₓ, KBr): 1728.9, 1555.7, 1266.5, 804.5 cm⁻¹.

LCMS (m/z): 525.0 (M+1, 100%), 527.1 (M+3, 96%).

### Step 3: Preparation of 3-[{4-[{benzo[1,3]dioxo-5-ylmethyl)amino]-6-[3-(2-methoxyphenyl)ureido]-[1,3,5]triazin-2-yl}-(3,4-dichloro-benzyl)amino]-propionic acid (Compound No. 97)

To the chlorotriazine (200 mg, 0.45 mmol) in THF (10 ml) was added piperonyl-amine (104 mg, 6.8 mmol) and triethylamine (93 mg, 0.9 mmol) and refluxed for 3 h. After cooling, the mixture was poured into cold water, acidified with HCl and extracted with EtOAc (2×50 ml). The combined organic extracts were washed with brine and dried anhydrous (Na₂SO₄) and concentrated. The residue was treated with hexane-dichloromethane (25:5 ml) and the white solid filtered and dried under vacuum to afford the title compound (141 mg, 53%).

m.p.: 120° C.

1HNMR (DMSO-d_{6,} 300 MHz): δ 11.17 (s) & 11.12 (s) [CO₂H] 10.88 (s) & 10.84 (s) [1H, NH], 9.59 (s), 9.47 (s) & 9.43 (s) [1, NH], 7.97 (1H, s) and 6.65-7.55 (10H, m) [aromatic], 5.96 (s), 5.93 (s) & 5.92 (s) [2H, OCH₂O], 4.83 (s) & 4.80 (s) [2H, NCH₂Ar], 4.47 (ABq, J=6 Hz), 4.38 (ABq, J=6 Hz) & 4.27 (ABq, J=6 Hz) [2H, NCH₂Ar] and 3.64-3.79 (5H, m, OCH₃ and NCH₂).

IR (νₘₐₓ, KBr): 1692.5, 1600.7, 1250.0, 1037.1, 750.3 cm⁻¹.

LCMS (m/z): 640.2 (M+1, 100%).

The following compounds were prepared similarly:

### Preparation of 3-[(4-[3-(2-Methoxyphenyl)ureido]-6-chloro-[1,3,5]triazin-2-yl)(3-methyl benzyl)amino]propionic acid (Compound No. 58)

### Step 1: Preparation of 3-[(4-Amino-6-chloro-[1,3,5]triazin-2-yl)-(3-methylbenzyl)-amino]propionic acid

3-[(4-Amino-6-chloro-[1,3,5]triazin-2-yl)-(3-methylbenzyl)-amino]propionic acid was prepared by using 3-(3-methylbenzyl)aminopropionic acid instead of 3-(3,4-dichlorobenzyl)aminopropionic acid in Step 1, Example 7.

m.p.: 168-170° C.

1HNMR (TFA-d, 300 MHz): δ 7.62 (2H, m) & 7.57 (2H, m) [aromatic] 5.39 (s) & 5.33 (s) [2H, NCH₂], 4.40 (t, J=6.9 Hz) & 4.32 (t, J=6.9 Hz) [2H, NCH₂], 3.17 (2H, t, J=6.9 Hz, CH₂CO₂H) and 2.74 (3H, s, Ar—CH₃).

IR (νₘₐₓ, KBr): 1713.0, 1568.4, 1321.5, 803.4, 777.9 cm⁻¹.

LCMS (m/z): 322.1 (M+1, 100%).

### Step 2: Preparation of 3-[(4-[3-(2-Methoxyphenyl)ureido]-6-chloro-[1,3,5]triazin-2-yl)(3-methyl benzyl)amino]propionic acid (Compound No. 58)

3-[(4-[3-(2-Methoxyphenyl)ureido]-6-chloro-[1,3,5]triazin-2-yl)(3-methyl benzyl)amino]propionic acid was prepared by using compound of Step 1 immediately above and 2-methoxyphenyl isocyanate as in Step 2, Example 7.

m.p.: 205° C.

1HNMR (DMSO-d_{6,} 300 MHz): δ 11.01 (s) & 10.91 (s) [1H, NH] 10.45 (1H, s, NH), 8.14 & 8.09 (1H, dd, J=14.1 Hz & 8.1 Hz), 7.23 & 7.13 (1H, t of d, J=17.1 and 8.1 Hz), 7.09 (bs) & 7.03 (bs) [5H] and 6.93 & 6.91 (1H, dd, J=7.8 and 3.9 Hz) [aromatic], 4.91 (s) & 4.84 (s) [2H, NCH₂Ar], 3.85 (s) & 3.81 (s) [3H, OCH₃], 3.65 (m, NCH₂), 2.57 (m, CH₂CO₂H) and 2.30 (s) & 2.22 (s) [3H, ArCH₃].

IR (νₘₐₓ, KBr): 1721.2, 1608.3, 1560.7, 1270.0, 743.4 cm⁻¹.

LCMS (m/z): 471.3 (M+1, 100%).

### 3-[(3,4-Dichlorobenzyl)-{4-isopropylamino-6-{3-(2-methoxyphenyl)ureido}-[1,3,5]triazin-2-yl}amino]propionic acid (Compound No. 80)

3-[(3,4-Dichlorobenzyl)-{4-isopropylamino-6-{3-(2-methoxyphenyl)ureido}-[1,3,5]triazin-2-yl}amino]propionic acid was prepared by using isopropylamine instead of piperonylamine in Step 3, Example 7.

m.p.: 95-101° C.

1HNMR (TFA-d, 300 MHz): δ 8.45-9.00 (7H, m, aromatic), 6.38 (s) & 6.35 (s) [2H, NCH₂Ar], 5.46 (bs), 5.34 (s) & 5.32 (s), [6H, OCH₃, NCH and NCH₂], 4.28 (2H, t, CH₂CO₂H] and 2.83 (d, J=6 Hz) & 2.72 (d, J=9 Hz) [6H, CH₃×2].

### 3-[(3,4-Dichlorobenzyl)-{4-(3-(2-methoxyphenyl)ureido)-6-(morpholin-4-yl) [1,3,5]triazin-2-yl}amino]propionic acid (Compound No. 81)

3-[(3,4-Dichlorobenzyl)-{4-(3-(2-methoxyphenyl)ureido)-6-(morpholin-4-yl) [1,3,5]triazin-2-yl}amino]propionic acid was prepared by using morpholine instead of piperonylamine in Step 3, Example 7.

m.p.: 198-203° C.

1HNMR (TFA-d, 300 MHz): δ 8.35-9.00 (7H, m, aromatic), 6.34 (2H, bs, NCH₂Ar), 5.46 (s), 5.39 (bs), 5.34 (bs) & 5.30 (bs) [13H, OCH₂×2, OCH₃, NCH₂×2 (ring) and NCH₂] and 4.34 (2H, t, CH₂CO₂H).

IR (νₘₐₓ, KBr): 1704.5, 1587.6, 1515.3, 1248.1, 750.4 cm⁻¹.

LCMS (m/z): 576.2 (M+1, 100%), 578.1 (M+3, 70%).

### 3-[(3,4-Dichlorobenzyl)-{4-(3-[2-methoxyphenyl)ureido]-6-(naphthalen-2-yl amino)-[1,3,5]triazin-2-yl}amino]-propionic acid (Compound No. 94)

3-[(3,4-Dichlorobenzyl)-{4-(3-[2-methoxyphenyl)ureido]-6-(naphthalen-2-yl amino)-[1,3,5]triazin-2-yl}amino]-propionic acid was prepared by using 2-aminonaphthalene instead of piperonylamine in Step 3, Example 7.

m.p.: 115-120° C.

1HNMR (DMSO-d_{6,} 300 MHz): δ 10.91 (1H, bs, NH), 9.73 (bs) & 9.66 (bs) [1H, NH], 6.92-7.95 (14H, m, aromatic), 4.98 (s) & 4.96 (s) [2H, NCH₂Ar], 3.4-3.85 (5H, m, OCH₃ and NCH₂) and 3.19 (2H, m, CH₂CO₂H).

IR (νₘₐₓ, KBr): 1712.4, 1687.0, 1441.5, 809.3, 742.3 cm⁻¹.

LCMS (m/z): 632.3 (M+1, 100%).

### 3-[{4-[(Benzo[1,3]dioxo-5-ylmethyl)amino]-6-[3-(2-methoxyphenyl)ureido]-[1,3,5]triazin-2-yl}-(3-methylbenzyl)amino]-propionic acid (Compound No. 84)

3-[{4-[(Benzo[1,3]dioxo-5-ylmethyl)amino]-6-[3-(2-methoxyphenyl)ureido]-[1,3,5]triazin-2-yl}-(3-methylbenzyl)amino]-propionic acid was prepared following the procedure of Example 7, by using Compound No. 58 and piperonylamine as in Step 3, Example 7.

m.p.: 165° C.

1HNMR (DMSO-d_{6,} 300 MHz): δ 11.14 (s) & 10.90 (s) [1H, NH], 9.53 (s) & 9.39 (s) [1H, NH], 7.97 (s) and 7.94 (s) [1H, NH], 6.68-7.60 (11H, m, aromatic), 5.96 (s) & 5.93 (s) [2H, OCH₂O], 4.84 (s) & 4.79 (s) [2H, NCH₂Ar], 4.45 (s) & 4.37 (ABq, J=5.1 Hz) [NCH₂Ar}, 3.48-3.76 (5H, m, OCH₃ and NCH₂), 2.56 (m, CH₂CO₂H) and 2.28 (s), 2.25 (s), 2.19 (s) & 2.07 (s) [3H, ArCH₃].

IR (νₘₐₓ, KBr): 685.2, 1596.6, 1251.7, 811.2, 747.2 cm⁻¹.

LCMS (m/z): 586.2 (M+1, 100%).

### 3-[{4-[3-(2-Methoxyphenyl)ureido]-6-[(thiophen-2-yl-methyl)amino]-[1,3,5]triazin-2-yl}-(3-methylbenzyl)amino]propionic acid (Compound No. 85)

3-[{4-[3-(2-Methoxyphenyl)ureido]-6-[(thiophen-2-yl-methyl)amino]-[1,3,5]triazin-2-yl}-(3-methylbenzyl)amino]propionic acid was prepared following the procedure of Example 7, by using Compound No. 58 and thiophen-2-yl-methylamine as in Step 3, Example 7.

m.p.: 89-95° C.

1HNMR (DMSO-d_{6,} 300 MHz): δ 10.93 (1H, bs, NH), 9.54 (1H, bs, NH), 7.35-8.05 (2H, m), 7.7 (1H, bs), 7.36 (1H, m), 7.18 (1H, t, J=7.5 Hz), 7.05 (5H, s) & 6.89 (2H, m) [aromatic], 4.87 (s), 4.76 (m), 4.66 (d, J=5.1 Hz) & 4.61 (m), [4H, NCH₂Ar×2), 3.85 (s), 3.79 (s) & 3.74 (s) [5H, OCH₃ and NCH2] and 2.20 (s) & 2.07 (s) [3H, ArCH₃].

IR (νₘₐₓ, KBr): 1690.5, 1595.5, 1429.3, 1251.7, 748.7 cm⁻¹.

LCMS (m/z): 548.2 (M+1, 100%).

### 3-[{4-(2,3-Dihydro-indol-1-yl)-6-[3-(2-methoxyphenyl)-ureido]-[1,3,5]triazin-2-yl}-(3-methylbenzyl)amino]propionic acid (Compound No. 86)

3-[{4-(2,3-Dihydro-indol-1-yl)-6-[3-(2-methoxyphenyl)-ureido]-[1,3,5]triazin-2-yl}-(3-methylbenzyl)amino]propionic acid was prepared following the procedure of Example 8, by using Compound No. 58 and indoline as in Step 3, Example 8.

m.p.: 201° C.

1HNMR (DMSO-d_{6,} 300 MHz): δ 10.77 (1H, s, CO₂H), 9.98 (bs) & 9.69 (bs) (1H, NH), 8.54 (bs) & 8.2 (bs) [1H, NH], 8.02 & 7.99 (1H, dd, J=6.3 and 5.1 Hz), 6.9-7.25 (11H, m) [aromatic], 4.94 (s) & 4.90 (s) [2H, NCH₂Ar] 4.21 (bs), 3.78 (bs). 3.77 (s) & 3.60 (bs) [7H, NCH₂×2 and OCH₃], 3.05 (2H, ABq. J=7.5 Hz, ArCH₂), 2.65 (2H, t, J=6.6 Hz, CH₂CO₂H) and 2.29 (s) & 2.22 (s) [3H, ArCH₃].

IR (νₘₐₓ, KBr): 1723.1, 1674.1, 1579.3, 1518.8, 1247.3, 747.8 cm⁻¹.

LCMS (m/z): 554.3 (M+1, 100%).

### 3-[{4-[3-(2-Methoxyphenyl)ureido]-6-piperidin-1-yl-[1,3,5]triazin-2-yl}-(3-methylbenzyl)amino]propionic acid (Compound No. 87)

3-[{4-[3-(2-Methoxyphenyl)ureido]-6-piperidin-1-yl-[1,3,5]triazin-2-yl}-(3-methylbenzyl)amino]propionic acid was prepared following the procedure of Example 7, by using Compound No. 58 and piperidine as in Step 3, Example 7.

m.p.: 91-92° C.

1HNMR (DMSO-d_{6,} 300 MHz): δ 10.82 (1H, s, NH), 9.41 (1H, s, NH), 7.98 (1H, t), 6.75-7.25 (7H, m) (aromatic), 4.86 (s) & 4.77 (s), (2H, NCH₂Ar), 3.79 (s), 3.74 (s) & 3.63 (t) [9H, NCH₂×3, OCH₃], 2.29 (s) & 2.21 (s) [3H, ArCH₃] and 1.62 (bs) & 1.52 (bs) [6H, CH₂×3].

IR (νₘₐₓ, KBr): 1711.8, 1589.0, 1512.1, 1254.1, 748.4 cm⁻¹.

LCMS (m/z): 520.1 (M+1, 100%).

### 3-[{4-[3-(2-Methoxyphenyl)ureido]-6-pyrrolidin-1-yl-[1,3,5]triazin-2-yl}-(3-methylbenzyl)amino]propionic acid (Compound No. 88)

3-[{4-[3-(2-Methoxyphenyl)ureido]-6-pyrrolidin-1-yl-[1,3,5]triazin-2-yl}-(3-methylbenzyl)amino]propionic acid was prepared by following the procedure of Example 7, by using Compound No. 58 and pyrrolidine as in Step 3, Example 7.

m.p.: 190° C.

1HNMR (DMSO-d_{6,} 300 MHz): δ 10.98 (1H, s, NH), 9.39 (1H, s, NH), 8.02 (1H, t), 7.19 (1H, t), 7.09 (5H, m) and 7.04 (1H, t) [aromatic], 4.83 (s) & 4.79 (s) [2H, NCH₂Ar], 3.79 (s), 3.77 (s) & 3.17-3.56 (m), [9H, OCH₃ and NCH₂×3], 2.56 (m, CH₂CO₂H), 2.29 (s) & 2.24 (s) [3H, ArCH₃] and 1.92 (4H, bs, CH₂×3).

IR (νₘₐₓ, KBr): 1715.8, 1589.7, 1512.5, 748.9 cm⁻¹.

LCMS (m/z): 506.1 (M+1, 100%).

### 3-[{4-[3-(2-Methoxyphenyl)ureido]-6-morpholin-4-yl-[1,3,5]triazin-2-yl}-(3-methylbenzyl)amino]propionic acid (Compound No. 89)

3-[{4-[3-(2-Methoxyphenyl)ureido]-6-morpholin-4-yl-[1,3,5]triazin-2-yl}-(3-methylbenzyl)amino]propionic acid was prepared following the procedure of Example 7, by using Compound No. 58 and morpholine as in Step 3, Example 7.

m.p.: 101-102° C.

1HNMR (DMSO-d_{6,} 300 MHz): δ 10.79 (1H, s, NH), 9.52 (1H, s, NH), 7.99 (1H, t, J=7.5 Hz), 7.18 (1H, m), 7.04 (5H, m) and 6.91 (1H, bs) [aromatic], 4.87 (s) and 4.79 (s) [2H, NCH₂Ar], 3.74 (s) & 3.67 (t, J=5.7 Hz) [13H, OCH₃, OCH₂×2 and NCH₂×3] and 2.29 (s) & 2.22 (s) [3H, ArCH₃].

IR (νₘₐₓ, KBr): 1710.5, 1588.8, 1515.2, 1248.7, 749.3 cm⁻¹.

LCMS (m/z): 522.1 (M+1, 100%).

### 3-[{4-Allylamino-6-[3-(2-methoxyphenyl)ureido]-[1,3,5]triazin-2-yl}-(3-methyl benzyl)amino]propionic acid (Compound No. 90)

3-[{4-Allylamino-6-[3-(2-methoxyphenyl)ureido]-[1,3,5]triazin-2-yl}-(3-methyl benzyl)amino]propionic acid was prepared following the procedure of Example 7, by using Compound No. 58 and allylamine as in Step 3, Example 7.

m.p.: 182-183° C.

1HNMR (DMSO-d_{6,} 300 MHz): δ 11.17 (s) & 10.91 (s) [1, NH], 9.44 (s) & 9.37 (s) [1H, NH], 7.61 (1H, bs, NH) 7.99 & 7.96 (1H, dd, J=7.2 Hz), 7.19 (1H, m), 7.03 (5H, m) and 6.90 (1H, bs) [aromatic], 5.83 (1H, m, olefinic CH), 5.13 (1H, t, J=17.7 Hz) & 5.04 (t, J=10.2 Hz) [olefinic CH₂], 4.86 (s) & 4.79 (s) [2H, NCH₂ Ar], 4.01 (bs) & 3.92 (bs), 3.79 (s), 3.74 (s) & 3.63 (m) [7H, NCH₂×2 and OCH₃] and 2.29 (s) & 2.19 (s) [3H, ArCH₃].

IR (νₘₐₓ, KBr): 1690.5, 1504.8, 1249.5, 812 cm⁻¹.

LCMS (m/z): 492.1 (M+1, 100%).

### 3-[(3,4-Dichlorobenzyl)-{4-[4-(2-isopropoxyphenyl)-piperazin-1-yl]-6-[3-(2-methoxyphenyl)ureido]-[1,3,5]triazin-2-yl}amino]-propionic acid (Compound No. 95)

3-[(3,4-Dichlorobenzyl)-{4-[4-(2-isopropoxyphenyl)-piperazin-1-yl]-6-[3-(2-methoxyphenyl)ureido]-[1,3,5]triazin-2-yl}amino]-propionic acid was prepared by using 4-(2-isopropoxyphenyl)piperazine instead of piperonyl-amine in Step 3, Example 7.

m.p.: 115-118° C.

1HNMR (DMSO-d_{6,} 300 MHz): δ 10.77 (s) & 10.75 (s) [1H, NH], 9.60 (s) & 9.57 (s) [1, NH], 8.03 & 7.99 (1H, dd, J=10.8 and 6.6 Hz), 7.58 (2H, t, J=9.6 Hz), 7.28 (1H, d, J=8.4 Hz), 7.04 (2H, s), 6.93 (4H, s) and 6.88 (1H, d, J=5.1 Hz) [aromatic] 4.86 (s) & 4.81 (s) [2H, NCH₂Ar], 4.63 (1H, bs, CHCH₃), 3.93 (bs), 3.82 (s), 3.79 (s) & 3.74 (t, J=6.6 Hz) [9H, OCH₃ and NCH₂×3], 3.05 (4H, m, NCH₂×2), 2.57 (2H, m, CH₂CO₂H) and 1.29 (d, J=5.1 Hz) & 1.27 (d, J=4.8 Hz) [6H, CH₃×2].

IR (νₘₐₓ, KBr): 1708.8, 1587.6, 1154.2, 1240.3, 748.8 cm⁻¹.

LCMS (m/z): 709.3 (M+1, 100%).

### 3-[(3,4-Dichlorobenzyl)-{4-[4-(2-methoxyphenyl)piperazin-1-yl]-6-[3-(2-methoxyphenyl)ureido]-[1,3,5]triazin-2-yl}amino]propionic acid (Compound No. 96)

I3-[(3,4-Dichlorobenzyl)-{4-[4-(2-methoxyphenyl)piperazin-1-yl]-6-[3-(2-methoxyphenyl)ureido]-[1,3,5]triazin-2-yl}amino]propionic acid was prepared by using 4-(2-methoxyphenyl)piperazine instead of piperonyl-amine in Step 3, Example 7.

1HNMR (DMSO-d_{6,} 300 MHz): δ 10.79 (s) & 10.77 (s) [1H, NH], 9.61 (s) & 9.58 (s) [1H, NH], 8.01 & 7.99 (1H, dd, J=7.2 Hz), 7.59 (2H, t, J=5.4 Hz), 7.28 (1H, d, J=8.4 Hz) 7.04 (s), 6.96 (s) & 6.90 (m) [7H] [aromatic], 4.86 (s) & 4.81 (s) [2H, NCH₂Ar], 3.92 (s), 3.82 (s), 3.81 (s), 3.79 (s) and 3.71 (t, J=7.2 Hz) [12H, OCH₃×2 and NCH₂×3], 3.03 (4H, m, NCH₂×2) and 2.57 (m, CH₂CO₂H).

IR (νₘₐₓ, KBr): 1719.8, 1591.6, 1511.9, 747.8 cm⁻¹.

LCMS (m/z): 681.2 (M+1, 100%).

### 3-[(3,4-Dichlorobenzyl)-{4-[4-[3-(2-methoxyphenyl)ureido]-6-[(thiophen-2-ylmethyl)amino]-[1,3,5]triazin-2-yl}amino]propionic acid (Compound No. 98)

3-[(3,4-Dichlorobenzyl)-{4-[4-[3-(2-methoxyphenyl)ureido]-6-[(thiophen-2-ylmethyl)amino]-[1,3,5]triazin-2-yl}amino]propionic acid was prepared by using thiophen-2-ylmethylamine instead of piperonylamine in Step 3, Example 7.

m.p.: 197° C.

1HNMR (DMSO-d_{6,} 300 MHz): δ 12.24 (1H, bs, CO₂H), 11.15 (s), 11.10 (s) & 10.88 (s) [1H, NH], 9.61 (s), 9.51 (s) & 9.49 (s) [1H, NH], 6.80-8.00 (11H, m, aromatic and 1 NH), 4.86 (s) & 4.05-4.82 (m) [4H, NCH₂Ar×2], 3.70-3.80 (5H, m, OCH₃ and NCH₂).

IR (νₘₐₓ, KBr): 1707.5, 1688.5, 1598.9, 812.5 cm⁻¹.

LCMS (m/z): 602.1 (M+1, 100%), 604.0 (M+3, 96%).

### 3-[(3,4-Dichlorobenzyl)-{4-(2,3-dihydro-indol-1-yl)-6-[3-(2-methoxyphenyl) ureido]-[1,3,5]triazin-2-yl}amino]propionic acid (Compound No. 99)

It 3-[(3,4-Dichlorobenzyl)-{4-(2,3-dihydro-indol-1-yl)-6-[3-(2-methoxyphenyl) ureido]-[1,3,5]triazin-2-yl}amino]propionic acid was prepared by using indoline instead of piperonylamine in Step 3, Example 7.

m.p.: 145-148° C.

1HNMR (DMSO-d_{6,} 300 MHz): δ 12.38 (1H, bs, CO₂H), 10.84 (1H, bs, NH), 9.89 (1H, bs, NH), 7.01-8.40 (1H, m, aromatic), 5.00 (2H, s, NCH₂ Ar), 3.26-4.31 (9H, m, OCH₃, NCH₂×2 and CH₂Ar) and 2.75 (m, CH₂CO₂H).

IR (νₘₐₓ, KBr): 1709.1, 1582.9, 1516.5, 749.7 cm⁻¹.

LCMS (m/z): 608.3 (M+1, 100%).

### 3-[(3,4-Dichlorobenzyl)-{4-[3-(2-methoxyphenyl)ureido]-6-piperidin-1-yl-[1,3,5]triazin-2-yl}amino)propionic acid (Compound No. 100)

3-[(3,4-Dichlorobenzyl)-{4-[3-(2-methoxyphenyl)ureido]-6-piperidin-1-yl-[1,3,5]triazin-2-yl}amino)propionic acid was prepared by using piperidine in place of piperonylamine in Step 3, Example 7.

m.p.: 111-115° C.

1HNMR (DMSO-d_{6,} 300 MHz): δ 12.28 (1H, bs, CO₂H), 10.77 (s), 10.76 (s), [1H, NH], 9.52 (s) & 9.47 (s) [1H, NH], 7.99 (1H, t), 7.56 (2H, t), 7.27 (1H, d, J=6.9 Hz), 7.03 (2H, s) & 6.92 (1H, s) [aromatic], 4.85 (s) & 4.78 (s) [NCH₂Ar], 3.79 (s), 3.77 (s) & 3.68 (m) [9H, OCH₃ and NCH₂×3] and 1.54 (6H, m, CH₂×3).

IR (νₘₐₓ, KBr): 1711.7, 1590.5, 1027.1, 748.4 cm⁻¹.

LCMS (m/z): 574.1 (M+1, 100%), 576.1 (M+3, 93%).

### 3-[(3,4-Dichlorobenzyl)-{4-[3-(2-methoxyphenyl)ureido]-6-pyrrolidin-1-yl-[1,3,5]triazin-2-yl}amino)propionic acid (Compound No. 101)

3-[(3,4-Dichlorobenzyl)-{4-[3-(2-methoxyphenyl)ureido]-6-pyrrolidin-1-yl-[1,3,5]triazin-2-yl}amino)propionic acid was prepared by using pyrrolidine instead of piperonylamine in Step 3, Example 7.

m.p.: 201-203° C.

1HNMR (DMSO-d_{6,} 300 MHz): δ 10.94 (1H, s, NH), 9.52 (s) & 9.49 (s) [1H, NH], 8.02 (1H, d, J=7.5 Hz), 7.59 (2H, d, J=7.84 Hz), 7.29 (1H, d, J=8.1 Hz), 7.03 (2H, d, J=3.6 Hz), 3.78 (s), 3.73 (s) & 3.70 (m) [9H, OCH₃ and NCH₂×3], 2.55 (2H, m, CH₂CO₂H) and 1.76 (4H, bs, CH₂×2).

IR (νₘₐₓ, KBr): 1702.9, 1589.0, 1509.5, 749.4 cm⁻¹.

LCMS (m/z): 560.1 (M+1, 100%).

### 3-[{4-Allylamino-6-[3-(2-methoxyphenyl)ureido]-[1,3,5]triazin-2-yl}-(3,4-dichlorobenzyl)-amino]propionic acid (Compound No. 102)

3-[{4-Allylamino-6-[3-(2-methoxyphenyl)ureido]-[1,3,5]triazin-2-yl}-(3,4-dichlorobenzyl)-amino]propionic acid was prepared by using allylamine instead of piperonylamine in Step 3, Example 7.

m.p.: 201° C.

1HNMR (DMSO-d_{6,} 300 MHz): δ 12.21 (1H, bs, CO₂H), 11.19 (s), 11.16 (s) & 10.86 (s) [1H, NH], 9.52 (s), 9.46 (s) & 9.43 (s) [1H, NH], 7.96 (1H, d, J=7.5 Hz), 7.54 (2H, m), 7.27 (1H, m), 7.02 (2H, s) & 6.91 (1H, m) [aromatic], 5.84 (1H, bs, olefinic CH), 4.9-5.20 (2H, m, olefinic CH₂), 4.85 (s) & 4.80 (s) [2H, NCH₂ Ar] and 4.01 (m), 3.92 (m), 3.79 (s) & 3.67 (m) [7H, OCH₃ and NCH₂×2].

IR (νₘₐₓ, KBr): 1712.3, 1688.2, 1546.3, 1259.1, 810.3 cm⁻¹.

LCMS (m/z): 546.0 (M+1, 100%).

### 1-{4-[(2-Carboxyethyl)-(3,4-dichlorobenzyl)amino]-6-[3-(2-methoxyphenyl) ureido]-[1,3,5]triazin-2-yl}-pyrrolidine-2S-carboxylic acid (Compound No. 113)

1-{4-[(2-Carboxyethyl)-(3,4-dichlorobenzyl)amino]-6-[3-(2-methoxyphenyl) ureido]-[1,3,5]triazin-2-yl}-pyrrolidine-2S-carboxylic acid was prepared by using pyrrolidine-2S-carboxylic acid (L-proline) instead of piperonyl-amine in Step 3, Example 7.

m.p.: 158° C.

1HNMR (DMSO-d_{6,} 300 MHz): δ 10.93 (1H, s, NH), 9.59 (s) & 9.56 (s), [1H, NH], 8.03 (t, J=6 Hz) & 7.86 (t) [1H], 7.57 (2H, m), 7.27 (1H, m), 7.04 (2H, s) & 6.92 (1H, s) [aromatic], 4.7-4.85 (2H, m, NCH₂Ar), 4.25-4.60 (1H, m, NCHCO₂H), 3.79 (s, OCH₃), 2.29 (2H, m, CH₂CO₂H) and 1.98 (4H, m, CH₂×2) (ring)).

IR (νₘₐₓ, KBr): 3449.3, 1718.2, 1587.7, 1027.9, 749.7 cm⁻¹.

LCMS (m/z): 604.2 (M+1, 35%).

### 3-[(3,4-Dichlorobenzyl)-{4-(4-hydroxypiperidinyl)-6-[3-(2-methoxyphenyl) ureido]-[1,3,5]triazin-2-yl}amino]propionic acid (Compound No. 123)

3-[(3,4-Dichlorobenzyl)-{4-(4-hydroxypiperidinyl)-6-[3-(2-methoxyphenyl) ureido]-[1,3,5]triazin-2-yl}amino]propionic acid was prepared by using 4-hydroxypiperidine instead of piperonylamine in Step 3, Example 7.

m.p.: 121-124° C.

1HNMR (TFA-d, 300 MHz): δ 8.25 (1H, bs), 8.07 (1H, d, J=9 Hz), 7.96 (2H, m) & 7.73 (3H, m), [aromatic], 5.54 (2H, s, NCH₂Ar) 4.93 (2H, bs), 4.50-4.75 (m), 4.55 (s) & 4.52 (s) [8H, OCH₃, NCHOH and NCH₂×3], 3.52 (2H, bs, CH₂CO₂H) and 2.25-2.80 (4H, m, CH₂ (ring)×2).

IR (νₘₐₓ, KBr): 1707.8, 1589.0, 1512.8, 808.1 cm⁻¹.

LCMS (m/z): 590.2 (M+1, 100%).

### 3-[{4-(4-Hydroxypiperidinyl-1-yl)-6-[3-(2-methoxyphenyl)ureido]-[1,3,5] triazin-2-yl}-(3-methylbenzyl)-amino]propionic acid (Compound No. 124)

3-[{4-(4-Hydroxypiperidinyl-1-yl)-6-[3-(2-methoxyphenyl)ureido]-[1,3,5] triazin-2-yl}-(3-methylbenzyl)-amino]propionic acid was prepared by following the procedure of Example 7, by using Compound No. 58 and 4-hydroxypiperidine as in Step 3, Example 7.

m.p.: 120-123° C.

1HNMR (TFA-d, 300 MHz): δ 8.38 (1H, bs), 7.89 (4H, m) & 7.72 (3H, m) [aromatic], 5.59 (s) & 5.50 (s) [2H, NCH₂Ar], 4.97 (bs) & 4.74 (t, J=6 Hz), 4.56 (s), 4.53 (s) & 4.30 (m) [10H, NCH, OCH₃ and NCH₂×3, 3.52 (t, J=9 Hz) & 3.47 (t) [2H, CH₂CO₂H] and 2.81 (2H, m) & 2.45 (2H, m) [CH₂ (ring)ₓ2].

IR (νₘₐₓ, KBr): 1715.8, 1590.3, 1513.1, 748.0 cm⁻¹.

LCMS (m/z): 536.3 (M+1, 100%).

### Example 8

### Preparation of 3-[(3,4-Dichlorobenzyl)-{6-[3-(2-methoxyphenyl)ureido]-4-oxo-4,5-dihydro-[1,3,5]triazin-2-yl}amino]propionic acid (Compound No. 92)

To the chlorotriazine (250 mg, 0.47 mmol) obtained from Step 2, Example 3 in THF-^{t}BuOH (9:1 ml) was added potassium tert-butoxide (160 mg, 1.42 mmol). The reaction mixture was refluxed for 6 hours and poured into cold water (50 ml). The mixture was acidified with concentrated. HCl and the solid that precipitated was filtered and dried in vacuum to obtain the title compound (110 mg, 46%) as a white solid.

m.p.: 188° C.

1HNMR (DMSO-d_{6,} 300 MHz): δ 8.02 (1H, d, J=9 Hz), 7.57 (2H, m), 7.28 (1H, s), 7.04 (2H, s) & 6.93 (1H, s) [aromatic], 4.81 (2H, s, NCH₂ Ar) and 3.83 (5H, s, OCH₃ and NCH₂).

IR (νₘₐₓ, KBr): 1717.7, 1514.1, 1029.3, 750.9 cm⁻¹.

LCMS (m/z): 507.1 (M+1, 100%).

The following compound was prepared similarly:

### 3-[{6-[3-(2-Methoxyphenyl)ureido]-4-oxo-4,5-dihydro-[1,3,5]triazin-2-yl}-(3-methylbenzyl)amino]propionic acid (Compound No. 83)

3-[{6-[3-(2-Methoxyphenyl)ureido]-4-oxo-4,5-dihydro-[1,3,5]triazin-2-yl}-(3-methylbenzyl)amino]propionic acid was prepared following the procedure of Example 8, and starting from Compound No. 58.

m.p.: 156° C.

1HNMR (DMSO-d_{6,} 300 MHz): δ 9.17 (1H, bs, NH), 8.00 (1H, bs, NH), 7.23 (1H, t). 7.09 (6H, s) & 6.94 (s) [aromatic], 4.83 (s) & 4.78 (s), (2H, NCH₂ Ar), 3.85 (s) & 3.82 (s) [3H, OCH₃], 3.59 (2H, m, NCH₂]), 2.55 (m, CH₂CO₂H) and 2.30 (s) & 2.27 (s) [3H, ArCH₃].

IR (νₘₐₓ, KBr): 3260.4, 1714.7, 1603.3, 1515.8, 749.9 cm⁻¹.

LCMS (m/z): 453.2 (M+1, 100%).

### 3-[{4-Methoxy-6-[3-(2-methoxyphenyl)ureido]-[1,3,5]triazin-2-yl}-(3-methyl benzyl)amino]propionic acid (Compound No. 91)

3-[{4-Methoxy-6-[3-(2-methoxyphenyl)ureido]-[1,3,5]triazin-2-yl}-(3-methyl benzyl)amino]propionic acid was prepared by following the procedure of Example 6, starting from Compound no. 58 and sodium methoxide.

m.p.: 160-162° C.

1HNMR (DMSO-d_{6,} 300 MHz): δ 11.25 (s) & 11.18 (s) [1H, NH], 9.98 (1H, s, NH) 8.08 (1H, t, J=8.7 Hz), 7.20 (1H, m), 7.0-7.15 (5H, m) & 6.91 (1H, t, J=3.6 Hz) [aromatic], 4.90 (s) & 4.85 (s) [2H, NCH₂Ar], 3.94 (s), 3.89 (s), 3.82 (s), 3.78 (s) & 3.70 (t, J=7.8 Hz) [8H, OCH₃×2 and NCH₂], 2.59 (m, CH₂CO₂H) and 2.29 (s) & 2.24 (s) [3H, ArCH₃].

IR (νₘₐₓ, KBr): 1709.8, 1596.8, 1530.5, 1255.0 cm⁻¹.

LCMS (m/z): 467.2 (M+1, 100%).

The following compound was prepared similarly:

### 3-[(3,4-Dichlorobenzyl)-{4-methoxy-6-[3-(2-methoxyphenyl)-ureido]-[1,3,5]triazin-2-yl}amino]propionic acid (Compound No. 103)

3-[(3,4-Dichlorobenzyl)-{4-methoxy-6-[3-(2-methoxyphenyl)-ureido]-[1,3,5]triazin-2-yl}amino]propionic acid was prepared by following the procedure of Example 6, and using Compound No. 59 and sodium methoxide.

m.p.: 208-210° C.

1HNMR (DMSO-d_{6,} 300 MHz): δ 11.16 (1H, bs, NH), 10.05 (1H, s, NH), 8.08 (1H, t, J=6 Hz), 7.60 (1H, d, J=6 Hz), 7.57 (1H, d, J=9 Hz), 7.31 (1H, t), 7.02 (2H, s) & 6.92 1H, m) [aromatic], 4.88 (s) & 4.86 (s) [2H, NCH₂Ar], 3.94 (s), 3.92 (s), 3.87 (s), 3.82 (s) & 3.80 (m) [8H, OCH₃×2 and NCH₂] and 2.58 (m, CH₂CO₂H).

IR (νₘₐₓ, KBr): 1708.5, 1596.2, 1253.5, 752.2 cm⁻¹.

LCMS (m/z): 521.2 (M+1, 100%).

### Example 9

### Primary Screening—Cell Adhesion Assay

VCAM-1 (100 ng/well) is coated in Maxisorp microtitre modules at 4° C. overnight. Non-specific blocking is carried out with 3% BSA for two hours and the wells washed with TBS (50 mM) Tris, 0.15M NaCl pH 7.4, 0.1 mM CaCl₂, 0.1 mM MgCl₂). U937 cells are suspended in fresh medium and incubated at 37° C. for two hours before the assay. Cells are then washed in TBS solution and 180 μI of cell suspension (1×10⁶ cells/ml in TBS buffer) is added per well in VCAM-1 coated wells. 20 μl of sample solution in 50% DMSO and 50% TBS is then added and the cells are incubated at 37° C. for one hour three to five dilutions of each sample are tested in duplicate in a primary screen, samples are tested at 1, 10 and 100 μm. If activity is present, the compounds are tested at lower (<1 μm) concentrates. After incubation, the non-adherent cells are removed by washing with TBS and the number of adhered cells are quantified by LDH activity estimation. The percent adhesion is calculated as compared to control.

The compounds disclosed herein for utility for the treatment of asthma and the symptoms of asthma, as well as for the treatment of multiple sclerosis, rheumatoid arthritis, allergic rhinitis, inflammatory bowel disease, and other cell adhesion—associated diseases and conditions and relief from the symptoms thereof.

We claim:

1. A compound having the structure of Formula I: 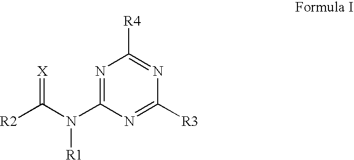
wherein
R₁ is hydrogen, alkyl, alkenyl, alkynyl, cycloalkyl, aralkyl, heteroaryl, heterocyclyl, heteroarylalkyl, or heterocyclylalkyl;
R₂ is alkyl, cycloalkyl, aryl, aralkyl, heteroaryl, heterocyclyl, heteroarylalkyl, heterocyclylalkyl, or —NR₅R₆ (wherein R₅ and R₆ are independently selected from hydrogen, alkyl, alkenyl, alkynyl, cycloalkyl, aryl, aralkyl, or SO₂R₁₂ [wherein R₁₂ is selected from the group alkyl, aryl, or heteroaryl], C(═O)R^{z} [wherein R^{z} is selected from the group alkyl, aralkyl, heteroarylalkyl, aryl, heteroaryl, or heterocyclyl], or C(═O)OR^{z} wherein R^{z} is the same as defined above, heterocyclyl, heteroaryl, heteroarylalkyl, heterocyclylalkyl);
X is oxygen atom, -sulphur atom, N(CN), N(NO₂) or CH(NO₂), provided that X is N(CN), N(NO₂) or CH(NO₂) when R₂ is NR₅R₆;
R₃ is halogen (F, Cl, Br and I), alkyl, alkenyl, alkynyl, cycloalkyl, hydroxyl, alkoxy, aryl, aralkyl, aryloxy, heterocyclyl, heteroaryl, heteroarylalkyl, heterocyclylalkyl, or NR₇R₈ (wherein R₇ and R₈ are independently selected from hydrogen, alkyl, alkenyl, alkynyl, cycloalkyl, aryl, aralkyl, biaryl, SO₂R₁₂ [wherein R₁₂ is selected from the group alkyl, aryl or heteroaryl], C(═O)R^{z} [wherein R^{z} is selected from the group alkyl, aralkyl, heteroarylalkyl, aryl, heteroaryl, or terocyclyl], C(═O)OR^{z} wherein R^{z} is the same as defined above, heteroaryl, heterocyclyl, heteroarylalkyl, or heterocyclylalkyl); and
R₄ is hydrogen, alkyl, alkenyl, alkynyl, aryl, aralkyl, halogen (F, Cl, Br and I), alkoxy, CH(CO₂R)₂ (wherein R represents hydrogen, alkyl, or aralkyl), heteroaryl, heterocyclyl, heteroarylalkyl, heterocyclylalkyl, NR₉R₁₀ (wherein R₉ and R₁₀ are independently selected from the group hydrogen, lower (C₁-C₆)alkyl, [further 1-2 hydrogens in the lower alkyl(C₁-C₆) may be substituted with (CH₂)ₜCO₂R₁₁ {wherein t is an integer in the range of 0-6 and R₁₁ is selected from the group consisting of hydrogen, alkyl, alkenyl, alkynyl, aralkyl, metal ions (Na⁺, K⁺, Ca²⁺ or Mg²⁺), ammonia, alkylated ammonia derivatives such as tris hydroxymethylaminomethyl}, aryl, aralkyl, heterocyclyl, heteroaryl, heteroarylalkyl, or heterocyclylalkyl], SO₂R₁₂ [wherein R₁₂ is selected from the group alkyl, aryl or heteroaryl], C(═O)R^{z} [wherein R^{z} is selected from the group alkyl, aralkyl, heteroarylalkyl, aryl, heteroaryl, or heterocyclyl], C(═O)OR^{z} wherein R^{z} is the same as defined above —(CH₂)ₘCO₂R₁₁ wherein m is an integer in the range varying from 1 to 6 R₁₁ is the same as defined earlier and further one hydrogen of CH₂ of —(CH₂)ₘCO₂R₁₁ may be substituted with heteroaryl or heterocyclyl], aryl, aralkyl, heteroaryl, heterocyclyl, heteroarylalkyl, or heterocyclylalkyl).

2. A pharmaceutical composition comprising a compound of claim 1 and pharmaceutically acceptable carrier.

3. A method of preventing, inhibiting or suppressing cell adhesion in an animal, comprising administration of the composition claim 2 to the animal.

4. A method of treating an inflammatory and/or auto immune disorder in an animal, comprising administration of the composition of claim 2 to the animal.

5. The method of claim 4, wherein the inflammatory and/or autoimmune disorder is at least one of bronchial asthma, rheumatoid arthristis, multiple sclerosis, type I diabetes, psoriasis, or allograft rejecting.

6. The method of preventing, inhibiting or suppressing cell adhesion—associated inflammation, immune or auto immune response in an animal comprising administration of a compound of claim 1.

7. A method of treating or preventing a disease selected from asthma, arthritis, psorasis, transplantation rejection, multiple sclorosis, diabetes, or inflammatory bowel disease in an animal comprising administration of a compound of claim 1.

8. A compound of claim 1 wherein
X is oxygen sulphur atom, N(CN), or CH(NO₂);
R₁ is hydrogen, alkyl or cycloalkyl; and
R₂ is NR₅R₆, wherein R₅ and R₆ are independently selected from hydrogen and substituted aryl.

9. A compound selected from
1-[4-Chloro-6-(napthalen-2-yloxy)-[1,3,5]triazin-2-yl]-3-(2-methoxyphenyl)urea (Compound 1);
1-[4-Chloro-6-(napthalen-2-yloxy)-[1,3,5]triazin-2-yl]-3-(2-chlorophenyl)urea (Compound 2);
1-[4-Chloro-6-(napthalen-2-yloxy-[1,3,5]triazin-2-yl]-3-[4-chlorophenyl)urea (Compound 3);
1-[4-Choro-6-(napthalen-2-yloxy)-[1,3,5]triazin-2-yl]-3-[4-methoxyphenyl)urea (Compound 4);
2-[4-[3-(2-Methoxyphenyl)ureido]-6-(napthalen-2-yloxy)-[1,3,5]triazin-2-yl]malonic acid-di-tert-butyl ester (Compound 5);
1-[4-Methoxy-6-(naphthalen-2-yloxy]-[1,3,5]triazin-2-yl]-3-(2-methoxyphenyl)urea (Compound 6);
4-[3-(2-Methoxyphenyl)ureido]-6-naphthalen-2-yloxy)-[1,3,5]triazin-2-yl]aminoacetic acid (Compound 7);
4-[3-(2-Chlorophenyl)ureido]-6(naphthalen-2-yloxy)-[1,3,5]triazin-2-yl]aminoacetic acid (Compound 8);
4-[3-(2-Methylphenyl)ureido]-6-(naphthalen-2-yloxy)-[1,3,5]triazin-2-yl]aminoacetic acid (Compound 9);
4-[3-(4-Methoxyphenyl)ureido]-6-(naphthalen-2-yloxy)-[1,3,5]triazin-2-yl]aminoacetic acid (Compound 10);
4-[3-(4-Chlorophenyl)ureido]-6-(naphthalen-2-yloxy]-[1,3,5]triazin-2-yl]aminoacetic acid (Compound 11);
4-[3-(4-Methylphenyl)ureido]-6-(naphthalen-2-yloxy]-[1,3,5]triazin-2-yl]aminoacetic acid (Compound 12);
2S-[4-[3-(2-Methoxyphenyl)-ureido]-6-(naphthalen-2-yloxy)-[1,3,5]triazin-2-ylamino}-3-phenylpropionic acid (Compound 13);
3-{4-[3-(2-Methoxyphenyl)ureido]-6-(napthalen-2-yloxy)-[1,3,5]triazin-2-ylamino}3-phenylpropoionic acid (Compound 14);
3-{Benzo[1,3]dioxo-5-yl-[4-[3-(2-methoxyphenyl) ureido]-6-napthalen-2-yloxy)-[1,3,5]triazin-2-ylamino) propionic acid ethyl ester (Compound 15);
3-(Benzo[1,3]dioxo-5-yl-([4-[3-(2-methoxyphenyl)-ureido]-6-napthalen-2-yloxy)-[1,3,5]triazin-2-ylamino} propionic acid (Compound 16);
1-[4-(Biphenyl-2-ylamino)-[1,3,5]triazin-2-yl-3-(2-methoxyphenyl)urea (Compound 17);
1-[4-(Biphenyl-2-ylamino)-[1,3,5]triazin-2-yl}-3-(2-methoxyphenyl)urea (Compound 18);
1-[4-(Biphenyl-2-ylamino)-6-methoxy-[1,3,5]triazin-2-yl}-3-(2-methoxyphenyl)urea (Compound 19);
{4-Biphenyl-2-ylamino)-6-[3-(2-methoxyphenyl)ureido]-[1,3,5]triazin-2-ylamino]acetic acid (Compound 20);
2S-{4-(Biphenyl-2-ylamino)-6-[3-(2-methoxyphenyl) ureido]-[1,3,5]triazin-2-ylamino}-3-phenylpropionic acid (Compound 21);
3-{4-Biphenyl-2-ylamino)-6-[3-(2-methoxyphenyl) ureido]-[1,3,5]triazin-2-ylamino}-3-phenylpropionic acid (Compound 22);
3-{3-Benzo[1,3]dioxo-5-yl-{4-(biphenyl-2-ylamino)-6-[3-(2-methoxyphenyl)ureido]-[1,3,5]triazin-2-ylamino} propionic acid (Compound 23);
2S-[4-(Biphenyl-2-ylamino)-6-[3-(2-methoxyphenyl) ureido]-[1,3,5]triazin-2-ylamino]-4-methylpentanoic acid (Compound 24);
2-{4-(Biphenyl-2-ylamino)-6-[3-(2-methoxyphenyl) ureido]-[1,3,5]triazin-2-ylamino}benzoic acid (Compound 25);
3-[{(Benzo[1,3]dioxo-5-ylmethyl-{4-(biphenyl-2-yl amino)-6-[3-(2-methoxyphenyl)ureido]-[1,3,5]triazin-2-yl}amino]propanoic acid (Compound 26);
3-[{4-(Biphenyl-2-ylamino)-6-[3-(2-methoxyphenyl) ureido]-[1,3,5]triazin-2-yl}-(3,4-dimethoxybenzyl) amino]-propionic acid (Compound 27);
3-[{4-(Biphenyl-2-ylamino)-6-[3-(2-methoxyphenyl) ureido]-[1,3,5]triazin-2-yl}-(4-methoxybenzyl)amino]-propionic acid (Compound 28);
3-[{4-(Biphenyl-2-ylamino)-6-[3-(2-methoxyphenyl) ureido]-[1,3,5]triazin-2-yl}-(3-methoxybenzyl)-amino] propionic acid (Compound 29);
3-[{4-(Biphenyl-2-ylamino)-6-[3-(2-methoxyphenyl)-ureido]-[1,3,5]triazin-2-yl}-(2-methoxybenzyl)amino] propionic acid (Compound 30);
3-[{4-(Biphenyl-2-ylamino]-6-[3-(2-methoxyphenyl) ureido]-[1,3,5]triazin-2-yl}-(3-methylbenzyl)amino] propionic acid (Compound 31);
3-[{4-(Biphenyl-2-ylamino)-6-[3-(2-methoxyphenyl) ureido]-[1,3,5]triazin-2-yl}-(4-methylbenzyl)amino] propionic acid (Compound 32);
3-[{4-(Biphenyl-2-ylamino)-6-[3-(2-methoxyphenyl) ureido-[1,3,5]triazin-2-yl}-(2-fluorobenzyl)amino] propionic acid (Compound 33);
3-[{4-(Biphenyl-2-ylamino]-6-[3-(2-methoxyphenyl) ureido]-[1,3,5]triazin-2-yl}-(4-fluorobenzyl)amino] propionic acid (Compound 34);
3-[{4-(Biphenyl-2-ylamino]-6-[3-(2-methoxyphenyl) ureido]-[1,3,5]triazin-2-yl}-(2-chlorophenyl)amino] propionic acid (Compound 35);
3-[{4-(Biphenyl-2-ylamino)-6-[3-(2-methoxyphenyl) ureido]-[1,3,5]triazin-2-yl}-(3,4-dichlorobenzyl)amino] propionic acid (Compound 36);
3-[{4-(Biphenyl-2-ylamino)-6-[3-(2-methoxyphenyl) ureido]-[1,3,5]triazin-2-yl}-(2,4-dichlorobenzyl)amino] propionic acid (Compound 37);
3-{4-(Biphenyl-2-ylamino]-6-[3-(2-methoxyphenyl) ureido]-[1,3,5]triazin-2-yl}amino}propionic acid (Compound 38);
[{4-(Biphenyl-2-ylamino)-6-[3-(2-methoxyphenyl) ureido-[1,3,5]triazin-2-yl}-(4-methylbenzyl)amino] acetic acid (Compound 39);
3-[{4-(Biphenyl-2-ylamino]-6-[3-(2-methoxyphenyl)-ureido-[1,3,5]triazin-2-yl}-(4-methoxybenzyl)amino] acetic acid (Compound 40);
[{4-(Biphenyl-2-ylamino)-6-[3-(2-methoxyphenyl) ureido]-[1,3,5]triazin-2-yl}-(3,4-dimethoxybenzyl) amino] acetic acid (Compound 41);
[{4-(Benzyl-2-ylamino)-6-[3-(2-methoxyphenyl)ureido]-[1,3,5]triazin-2-yl}-[4-fluorobenzyl]amino]acetic acid (Compound 42);
3-{4-[3-(2-Methoxyphenyl)ureido]-6-(naphthalen-2-yloxy)-[1,3,5]triazin-2-ylamino}propionic acid (Compound 43);
3-{(3-Methoxybenzyl)-[4-[3-(2-methoxyphenyl)ureido]-6-(naphthalen-2-yloxy)-[1,3,5]triazin-2-yl]amino} propionic acid (Compound 44);
3-{(4-Methoxybenzyl)-[4-[3-(2-methoxyphenyl)ureido]-6-[naphthalen-2-yloxy)-[1,3,5]triazin-2-yl]amino} propionic acid (Compound 45);
3-{(4-Fluorobenzyl)-[4-[3-(2-methoxyphenyl)-ureido]-6-[naphthalen-2-yloxy)-[1,3,5]triazin-2-yl]amino} propionic acid (Compound 46);
3-{(4-Methylbenzyl)-[4-[3-(2-methoxyphenyl)ureido]-6-[naphthalen-2-yloxy)-[1,3,5]triazin-yl]amino}propionic acid (Compound 47);
Tris salt of 3-[{4-(Biphenyl-2-ylamino)-6-[3-(2-methoxyphenyl) ureido]-[1,3,5]triazin-2-yl}-(3-methylbenzyl)amino] propionic acid (Compound 48);
Na salt of 3-[{4-(Biphenyl-2-ylamino)-6-[3-(2-methoxyphenyl)-ureido]-[1,3,5]triazin-2-yl}-(3-methylbenzyl)amino] propionic acid (Compound 49);
3-{Benzo[1,3]dioxo-5-ylmethyl-[4-[3-(2-methoxyphenyl)-ureido]-6-(napthanalene-2-yloxy)-[1,3,5]triazin-2-yl]amino}propionic acid (Compound 50);
3-{2-Methoxybenzyl)-[4-[3-(2-methoxyphenyl)ureido]-6-(naphthalen-2-yloxy)-[1,3,5]triazin-2-yl]amino} propionic acid (Compound 51);
3-{(3,4-Dimethoxybenzyl)-[4-[3-(2-methoxyphenyl) ureido-6-(naphthalen-2-yloxy)-[1,3,5]triazin-2-yl]amino}propionic acid (Compound 52);
3-{(3-Methylbenzyl)-[4-[3-2-(methoxyphenyl)ureido-6-(naphthalen-2-yloxy)-[1,3,5]triazin-2-yl]amino} propionic acid (Compound 53);
3-{(2-Fluorobenzyl)-[4-[3-2-(methoxyphenyl)ureido-6-(naphthalen-2-yloxy)-[1,3,5]triazin-2-yl]amino} propionic acid (Compound 54);
3-{(2-Chlorobenzyl)-[4-[3-2-methoxyphenyl)ureido-6-(naphthelen-2-yloxy)-[1,3,5]triazin-2-yl]amino] propionic acid (Compound 55);
3-{(2,4-Dichlorobenzyl)-[4-[3-2-methoxyphenyl)ureido-6-(naphthalen-2-yloxy)-[1,3,5]triazin-2-yl]amino} propionic acid (Compound 56);
3-{3,4-Dichlorobenzyl)-[4-[3-2-methoxyphenyl)ureido-6-(naphthalen-2-yloxy)-[1,3,5]triazin-2-yl]amino} propionic acid (Compound 57);
3-[(4-[3-(2-Methoxyphenyl)ureido]-6-chloro-[1,3,5] triazin-2-yl)(3-methyl-benzyl)amino]propionic acid (Compound 58);
3-[{4-(2-methoxyphenyl)ureido)-6-chloro-[1,3,5]triazin-2-yl}-(3,4-dichlorobenzyl)amino]propionic acid (Compound 59);
3-{(1H-Indol-3-yl)-2S([4-(2-methoxypheyl)ureido]-6-(naphthalen-2-yloxy-[1,3,5]triazin-2-yl)amino]propionic acid (Compound 60);
2-{4-[3-(2-Methoxyphenyl)ureido]-6-(naphthalen-2-yloxy)-[1,3,5]triazin-2-yl)}1,2,3,4-tetrahydro-isoquinoldine-3-carboxylic acid (Compound 61);
2-{(4-Fluorobenzyl)-[4-[3-(2-methoxyphenyl)ureido-6-(naphthalen-2-yloxy)-[1,3,5]triazin-2-yl]amino}-acetic acid (Compound 62);
2-{(4-Methoxybenzyl)-[4-[3-(2-methoxyphenyl)ureido-6-(naphthalen-2-yloxy)-[1,3,5]triazin-2-yl]amino}acetic acid (Compound 63);
2-{(4-Methylbenzyl)-[4-[3-(2-methoxyphenyl)ureido-6-(naphthalen-2-yloxy)-[1,3,5]triazin-2-yl]amino}acetic acid (Compound 64);
3-{3,4-Difluorobenzyl)-[4-(3-(2-methoxyphenyl)ureido-6-naphthalen-2-yloxy)-[1,3,5]triazin-2-yl]amino} propanoic acid (Compound 65);
3-{[4-[3-(2-Methoxyphenyl)ureido]-6-(naphthalen-2-yloxy)-[1,3,5]triazin-2-yl]-thiophen-2-yl-methylamino} propionic acid (Compound 66);
3-{(4-Trifluoromethylbenzyl)-[4-[3-(2-methoxyphenyl) ureido-6-(naphthalen-2-yloxy)-[1,3,5]triazin-2-yl]amino}propanoic acid (Compound 67);
3-{[2-(1H-Indol-3-yl)ethyl]-[4-[3-(2-methoxyphenyl) ureido]-6-(naphthalen-2-yloxy)-[1,3,5]triozin-2-yl]amino]propionic acid (Compound 68);
1-[4-[2-(4-Hydroxyphenyl)ethylamino]-6-(naphthalen-2-yloxy)-[1,3,5]triazin-2-yl]-3-(2-methoxyphenyl)urea (Compound 69);
1-[4-2-(1H-Indol-3-yl)ethylamino]-6-(naphthalen-2-yloxy)-[1,3,5]triazin-2-yl]-3-(2-methoxyphenyl)urea (Compound 70);
1-(2-Methoxyphenyl)-3-[(4-(3-methylbenzyl)amino)-6-(naphthalen-2-yloxy)-[1,3,5]triazin-2-yl]urea (Compound 71);
1-[(4-(3,4-Dichlorobenzyl)amino)-6-(naphthalen-2-yloxy)-[1,3,5]triazin-2-yl]-3-(2-methoxyphenyl)urea (Compound 72);
3-{4-(Biphenyl-2-ylamino)-6-[3-(2-methoxyphenyl) ureido]-[1,3,5]triazin-2-yl}-(4-trifluoromethyl benzyl) amino]propionic acid (Compound 73);
3-[{4-(Biphenyl-2-ylamino)-6-[3-(2-methoxyphenyl) ureido]-[1,3,5]triazine-2-yl}-(thiophen-2-ylmethyl) amino]propionic acid (Compound 74);
3-[{4-(Biphenyl-2-ylamino)-6-[3-(2-methoxyphenyl) ureido]-[1,3,5]triazin-2-yl}-(3,4-diflourobenzyl)amino] propionic acid (Compound 75);
3-[{4-(Biphenyl-2-ylamino)-6-[3-(2-methoxyphenyl) ureido]-[1,3,5]trazin-2-yl}-(2-((4-hydroxyphenyl)ethyl) amino]propionic acid (Compound 76);
3-[{4-(Biphenyl-2-ylamino)-6-[3-(2-methoxyphenyl) ureido)-[1,3,5]triazin-2-yl}-[2-(1H-indol-3-yl)ethyl]amino]propionic acid (Compound 77);
4-[{4-(Biphenyl-2-ylamino)-6-[-(2-methoxyphenyl) ureido]-[1,3,5]triazin-2-yl]amino}phenylacetic acid (Compound 78);
4-[{4-{3-(2-Methoxyphenyl)ureido}-6-(naphthalen-2-yloxy)-[1,3,5]triazin-2-yl}amino]phenylacetic acid (Compound 79);
3-{(3,4-Dichlorobenzyl)-{4-isopropylamine-6-[3-(2-methoxyphenyl)ureido}[1,3,5]triazin-2-yl}amino] propionic acid (Compound 80);
3-((3,4-Dichlorobenzyl)-{4-(3-(2-methoxyphenyl) ureido)-6-(morpholin-4-yl)[1,3,5]triazin-2-yl}amino} propionic acid (Compound 81);
3-[{4-(Biphenyl-4-ylamino)-6-[3-(2-methoxyphenyl) ureido]-[1,3,5]triazin-2-yl}-(3-methylbenzyl)amino] propionic acid (Compound 82);
3-[{6-[3-(2-Methoxyphenyl)ureido]-4-oxo-4,5-dihydro-[1,3,5]triazin-2-yl}-(3-methylbenzyl)amino]propionic acid (Compound 83);
3-[{4-[(Benzo[1,3]dioxo-5-ylmethyl)amino]-6-[3-(2-methoxyphenyl)ureido]-[1,3,5]triazin-2-yl}-(3-methylbenzyl)amino]-propionic acid (Compound 84);
3-[{4-[3-(2-Methoxyphenyl)ureido]-6-[(thiophen-2-yl-methyl)amino]-[1,3,5]triazin-2-yl}-(3-methylbenzyl) amino]propionic acid (Compound 85);
3-[{4-(2,3-Dihydro-indol-1-yl)-6-[3-(2-methoxyphenl)-ureido]-[1,3,5]triazin-2-yl}-(3-methylbenzyl)amino] propionic acid (Compound 86);
3-[(4-[3-(2-Methoxyphenyl)ureido]-6-piperidin-1-yl-[1,3,5]triazin-2-yl}-(3-methyl-benzyl)amino]propionic acid (Compound 87);
3-[{4-[3-(2-Methoxyphenyl)ureido]-6-pyrrolidin-1-yl-[1,3,5]triazin-2-yl}-(3-methylbenzyl)amino]propionic acid (Compound 88);
3-[{4-[3-(2-Mehoxyphenyl)ureido]-6-morpholin-4-yl-[1,3,5]triazin-2-yl}-(3-methylbenzyl)amino]propionic acid (Compound 89);
3-[{4-Allylamino-6-[3-(2-methoxyphenyl)ureido]-[1,3,5] triazin-2-yl}-(3-methylbenzyl)amino]propionic acid (Compound 90);
3-[{4-Methoxy-6-[3-(2-methoxyphenyl)ureido]-[1,3,5] triazin-2-yl}-(3-methylbenzyl)amino]propionic acid (Compound 91);
3-[(3,4-Dichlorobenzyl)-{6-[3-(2-methoxyphenyl) ureido]-4-oxo-4,5-dihydro-[1,3,5]triazin-2-yl}amino) propionic acid (Compound 92);
3-[{4-(Biphenyl-4-ylamino)-6-[3-(2-methoxyphenyl) ureido]-[1,3,5]triazin-2-yl}-(3,4-dichlorobenzyl)amino] propionic acid (Compound 93);
3-{(3,4-Dichlorobenzyl)-[4-(3-[2-methoxyphenyl) ureido]-6-(naphthalen-2-ylamino)-[1,3,5]triazin-2-yl}amino]-propionic acid (Compound 94);
3-((3,4-Dichlorobenzyl)-{4-[4-(2-isopropoxyphenyl)-piperazin-1-yl]-6-[3-(2-methoxyphenyl)ureido]-[1,3,5] triazin-2-yl}amino)-propionic acid (Compound 95);
3-((3,4-Dichlorobenzyl)-{4-[4-(2-methoxyphenyl) piperazin-1-yl]-6-[3-(2-methoxyphenyl)ureido]-[1,3,5] triazin-2-yl}amino] propionic acid (Compound 96);
3-[{4-[{benzo[1,3]dioxo-5-ylmethyl)amino]-6-[3-(2-methoxyphenyl)ureido]-[1,3,5]triazin-2-yl}-(3,4-dichloro-benzyl)amino]propionic acid (Compound 97);
3-((3,4-Dichlorobenzyl)-{4-[4-[3-(2-methoxyphenyl) ureido]-6-[(thiophen-2-ylmethyl)amino]-[1,3,5]triazin-2-yl}amino]propionic acid (Compound 98);
3-[(3,4-Dichlorobenzyl)-{4-(2,3-dihydro-indol-1-yl)-6-[3-(2-methoxyphenyl)ureido]-[1,3,5]triazin-2-yl}amino] propionic acid (Compound 99);
3-((3,4-Dichlorobenzyl)-{4-[3-(2-methoxyphenyl) ureido]-6-piperidin-1-yl-[1,3,5]triazin-2-yl}amino) propionic acid (Compound 100);
3-[(3,4-Dichlorobenzyl)-{4-[3-(2-methoxyphenyl) ureido]-6-pyrrolidin-1-yl-[1,3,5]triazin-2-yl}amino) propionic acid (Compound 101);
3-{[{4-Allylamino-6-[3-(2-methoxyphenyl)ureido]-[1,3,5]triazin-2-yl}-(3,4-dichlorobenzyl)-amino] propionic acid (Compound 102);
3-((3,4-Dichlorobenzyl)-{4-methoxy-6-[3-(2-methoxy phenyl)-ureido]-[1,3,5]triazin-2-yl}amino]propionic acid (Compound 103);
3-{[4-[3-(2-Methoxyphenyl)ureido]-6-(naphthalen-2-yloxy)-[1,3,5]triazin-2-yl]-methylamino}-propionic acid (Compound 104);
3-{(3-Fluorobenzyl)-[4-[3-(2-methoxyphenyl)ureido]-6-(naphthalen-2-yloxy)-[1,3,5]triazin-2-yl]amino} propionic acid (Compound 105);
3-{(3-Chlorobenzyl)-[4-[3-(2-methoxyphenyl)ureido]-6-(napthalen-2-yloxy)-[1,3,5]triazin-2-yl]amino}propionic acid (Compound 106);
3-{[4-[3-(2-Methoxyphenyl)ureido]-6-(napthalen-2-yloxy]-[1,3,5]triazin-2-yl]-(2-trifluoromethylbenzyl) amino}propionic acid (Compound 107);
3-{[4-[3-(2-Methoxyphenyl)ureido]-6-(naphthalen-2-yloxy)-[1,3,5]triazin-2-yl]-(3-trifluoromethylbenzyl) aminopropionic acid (Compound 108);
3-{(2,4-Bis-trifluoromethylbenzyl}-[4-[3-(2-methoxy phenyl)ureido]-6-(naphthalen-2-yloxy)-[1,3,5] triazin-2-yl]amino}propionic acid (Compound 109);
3-{(Biphenyl-4-ylmethyl)-[4-[3-(2-methoxyphenyl) ureido]-6-(naphthalen-2-yloxy)-[1,3,5]triazin-2-yl]amino}propionic acid (Compound 110)
3-{[4-[3-(2-Methoxyphenyl)ureido]-6-(naphthalen-2-yloxy)-[1,3,5]triazin-2-yl]-(naphthalen-1-ylmethyl) amino}-propionic acid (Compound 111);
3-[{4-(Biphenyl-2-ylamino)-6-[3-(2-hydroxyphenyl) ureido]-[1,3,5]triazin-2-yl}-(3-methylbenzyl)amino] propionic acid (Compound 112);
1-{4-[(2-Carboxyethyl)-(3,4-dichlorobenzyl)amino]-6-[3-(2-methoxyphenyl)ureido]-[1,3,5]triazin-2-yl}-pyrrolidine-2S-carboxylic acid (Compound 113);
3-[{4-(Biphenyl-2-ylamino)-6-[3-(2-methoxyphenyl) ureido]-[1,3,5]triazin-2-yl}-(3-fluorobenzyl)amino] propionic acid (Compound 114);
3-[{4-(Biphenyl-2-ylamino)-6-[3-(2-methoxyphenyl) ureido]-[1,3,5]triazin-2-yl}-(3-chlorobenzyl)amino] propionic acid (Compound 115);
3-[{4-(Biphenyl-2-ylamino)-6-[3-(2-methoxyphenyl) ureido][1,3,5]triazin-2-yl]-(2-trifluoromethylbenzyl) amino]propionic acid (Compound 116);
3-[{4-(Biphenyl-2-ylamino)-6-[3-(2-methoxyphenyl) ureido]-[1,3,5]triazin-2-yl}-(3-trifluoromethylbenzyl) amino]propionic acid (Compound 117);
3-[{4-(Biphenyl-2-ylamino)-6-[3-(2-methoxyphenyl) ureido]-[1,3,5]triazin-2-yl}-(3,5-bis-trifluoromethyl benzyl)amino]propionic acid (Compound 118);
3-({4-(Biphenyl-2-ylamino)-6-[3-(2-methoxyphenyl) ureido]-[1,3,5]triazin-2-yl}-(biphenyl-4-ylmethyl) amino]propionic acid (Compound 119);
3-({4-(Biphenyl-2-ylamino)-6-[3-(2-methoxyphenyl) ureido]-[1,3,5]triazine-2-yl}-(naphthalen-1-ylmethyl) amino]propionic acid (Compound 120);
3-[{4-(Biphenyl-2-ylamino)-6-[3-(2-methoxyphenyl) ureido]-[1,3,5]triazin-2-yl}-(2-pyridin-2-ylethyl)amino] propionic acid (Compound 121);
1-{4-(Biphenyl-2-ylamino}-6-[2-cyanoethyl)-(3-methylbenzyl)-amino]-[1,3,5]triazin-2-yl}-3-(2-methoxyphenyl)urea (Compound 122);
3-((3,4-Dichlorobenzyl)-{4-(4-hydroxypiperidinyl)-6-[3-(2-methoxyphenyl)ureido]-[1,3,5]triazin-2-yl}amino] propionic acid (Compound 123);
3-[{4-(4-Hydroxypiperidinyl-1-yl)-6-[3-(2-methoxy phenyl)ureido]-[1,3,5]triazin-2-yl}-(3-methylbenzyl)-amino]propionic acid (Compound 124);
1-{4-(Biphenyl-2-ylamino)-6-[3-(2-methoxyphenyl) ureido-[1,3,5]triazin-2-yl}-pyrrolidine-2S-carboxylic acid (Compound 125);
1-{4-(Biphenyl-2-ylamino)-6-[3-(2-methoxyphenyl) ureido]-[1,3,5]triazin-2-yl}-thiazolidine-4-carboxylic acid (Compound 126);
1-{4-(Biphenyl-2-yl-amino)-6-[3-(2-methoxyphenyl) ureido]-[1,3,5]triazin-2-yl}-piperidine-3-carboxylic acid (Compound 127);
1-{4-(Biphenyl-2-yl-amino)-6-[3-(2-methoxyphenyl) ureido]-[1,3,5]triazin-2-yl}-piperidine-4-carboxylic acid (Compound 128);
2-{4-(Biphenyl-2-yl-amino)-6-[3-(2-methoxyphenyl) ureido]-[1,3,5]triazin-2-yl}-1,2,3,4-tetrahydro-isoquinoline-3-carboxylic acid (Compound 129); and
1-{4-(Biphenyl-2-ylamino)-6-[3-(2-methoxyphenyl) ureido]-[1,3,5]triazin-2-yl}piperidine-2-carboxylic acid (Compound 130).

10. A method of making a compound of Formula VII, 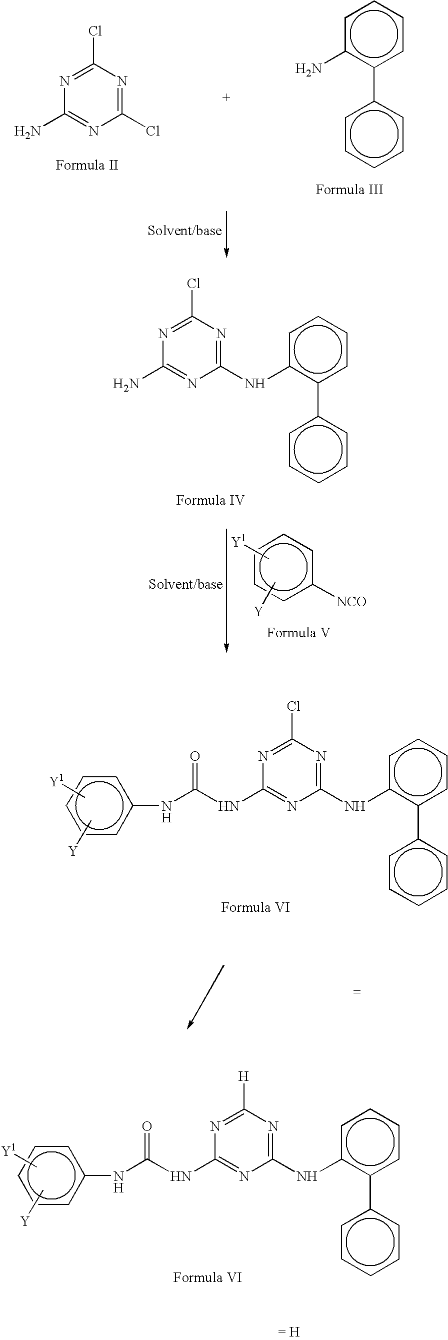
wherein Y and Y′ are independently hydrogen, alkyl, alkoxy, or halogen, the method comprising:
reacting a compound of Formula II with a compound of Formula III to give a compound of Formula IV;
reacting the compound of Formula IV with a compound of Formula V to give a compound of Formula VI; and
hydrogenating the compound of Formula VI to give the compound of Formula VII.

11. A method of making a compound of Formula VIII, 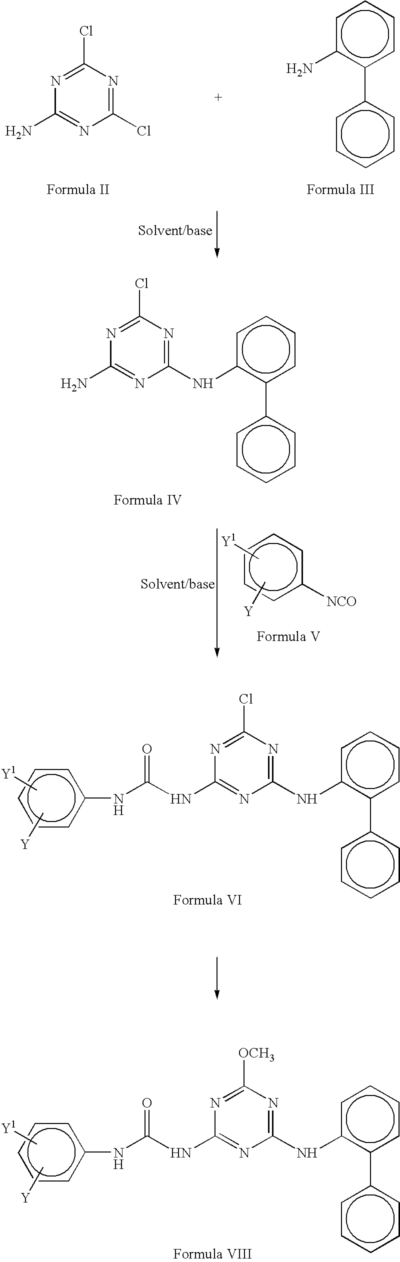
wherein Y and Y′ are independently hydrogen, alkyl, alkoxy, or halogen, the method comprising:
reacting a compound of Formula II with a compound of Formula III to give a compound of Formula IV;
reacting the compound of Formula IV with a compound of Formula V to give a compound of Formula VI; and
reacting the compound of Formula VI with an alkali metal methoxide to give the compound of Formula VIII.

12. A method of making a compound of Formula X, 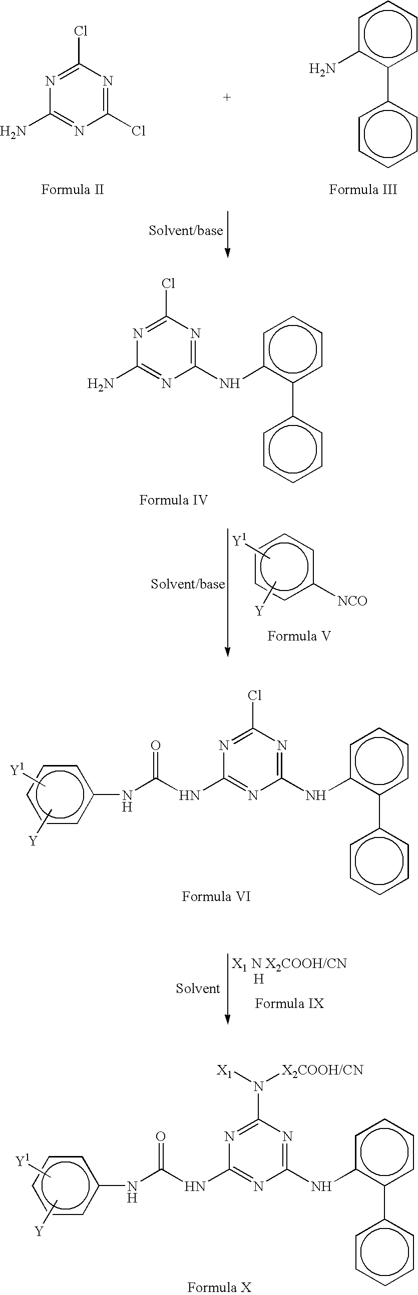
wherein Y and Y′ are independently hydrogen, alkyl, alkoxy, or halogen, and X₁ and X₂ are independently hydrogen, substituted alkyl or substituted aryl; the method comprising:
reacting a compound of Formula II with a compound of Formula III to give a compound of Formula IV;
reacting the compound of Formula IV with a compound of Formula V to give a compound of Formula VI; and
reacting the compound of Formula VI with a compound of Formula IX to give the compound of Formula X.

13. A method of making a compound of Formula XIV, 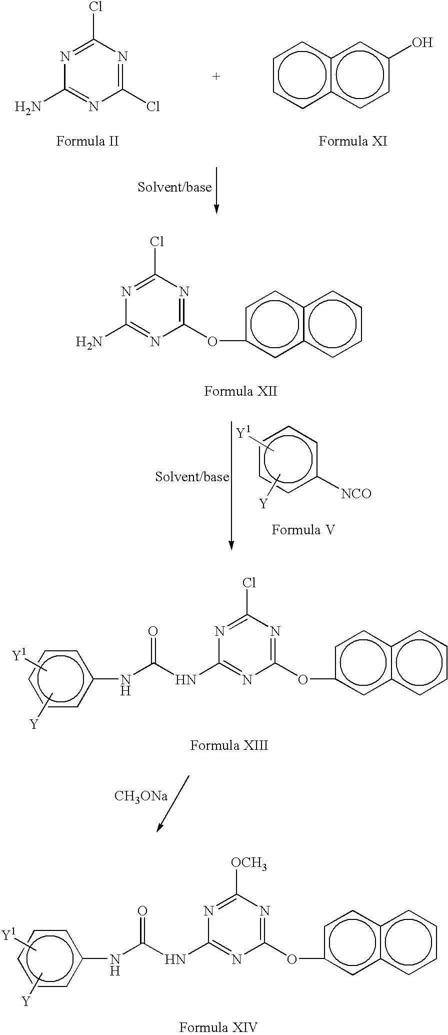
wherein Y and Y′ are independently hydrogen, alkyl, alkoxy, or halogen, the method comprising:
reacting a compound of Formula II with a compound of Formula XI to give a compound of Formula XII;
reacting the compound of Formula XII with a compound of Formula V to give a compound of Formula XIII; and
reacting the compound of Formula XIII with an alkali metal methoxide to give the compound of Formula XIV.

14. A method of making a compound of Formula XV, 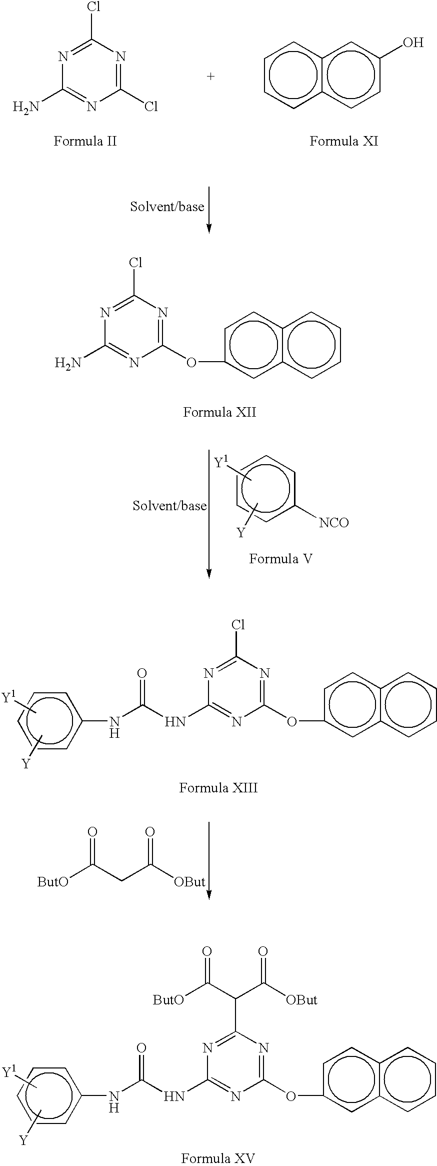
wherein Y and Y′ are independently hydrogen, alkyl, alkoxy, or halogen, the method comprising:
reacting a compound of Formula II with a compound of Formula XI to give a compound of Formula XII;
reacting the compound of Formula XII with a compound of Formula V to give a compound of Formula XIII; and
reacting the compound of Formula XIII with dibutylmalonate to give the compound of Formula XV.

15. A method of making a compound of Formula XVI, 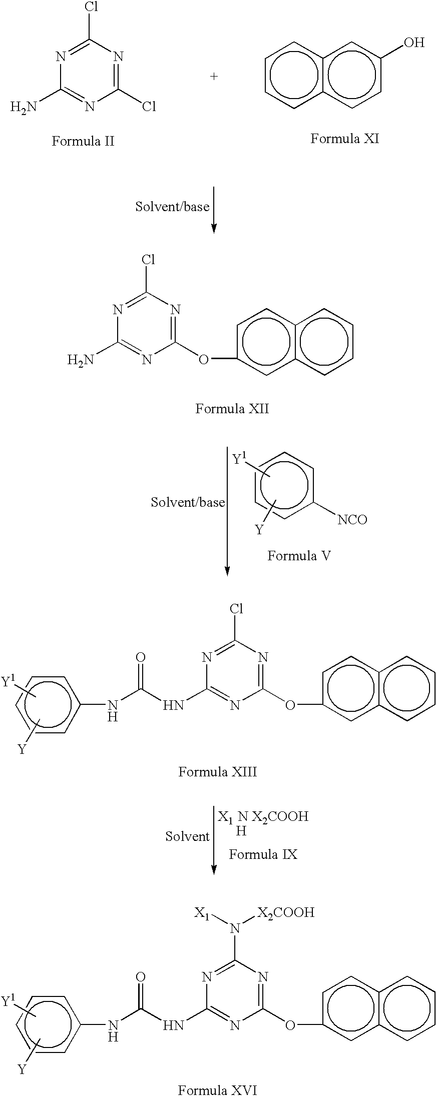
wherein Y and Y′ are independently hydrogen, alkyl, alkoxy, or halogen, and X₁ and X₂ are independently hydrogen, substituted alkyl or substituted aryl, the method comprising:
reacting a compound of Formula II with a compound of Formula XI to give a compound of Formula XII;
reacting the compound of Formula XII with a compound of Formula V to give a compound of Formula XIII; and
reacting the compound of Formula XIII with a compound of Formula IX to give the compound of Formula XVI.

16. A method of making a compound of Formula XIX, 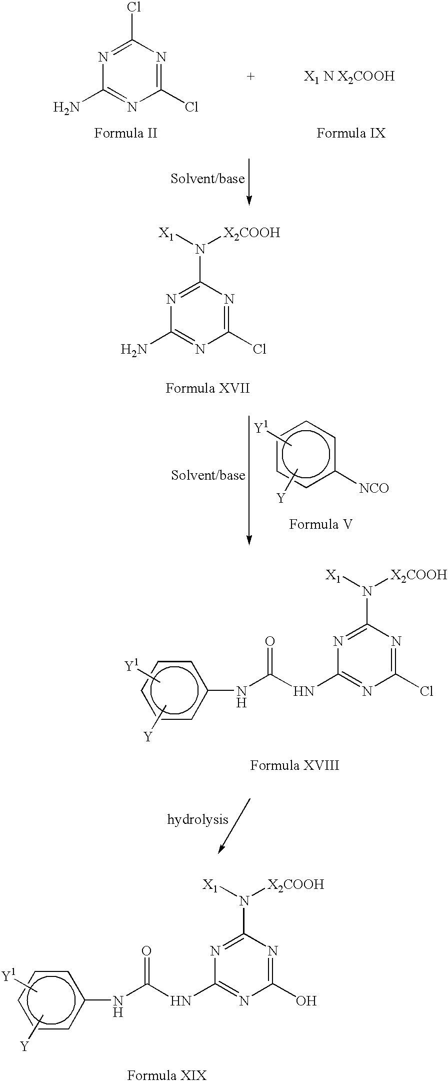
wherein Y and Y′ are independently hydrogen, alkyl, alkoxy, or halogen, and X₁ and X₂ are independently hydrogen, substituted alkyl or substituted aryl, the method comprising:
reacting a compound of Formula II with a compound of Formula IX to give a compound of Formula XVII;
reacting the compound of Formula XVII with a compound of Formula V to give a compound of Formula XVIII; and
hydrolyzing the compound of Formula XIII to give the compound of Formula XIX.

17. A method of making a compound of Formula XX, 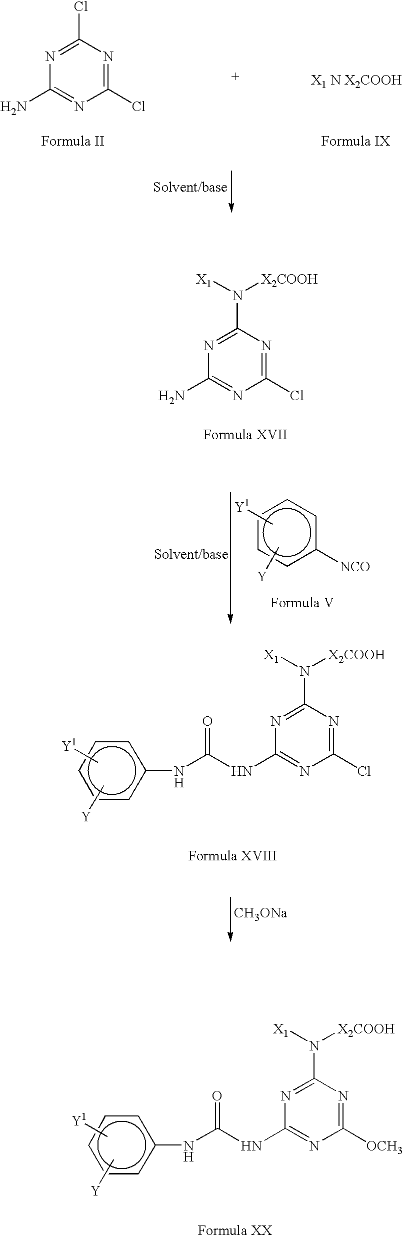
wherein Y and Y′ are independently hydrogen, alkyl, alkoxy, or halogen, and X₁ and X₂ are independently hydrogen, substituted alkyl or substituted aryl, the method comprising:
reacting a compound of Formula II with a compound of Formula IX to give a compound of Formula XVII;
reacting the compound of Formula XVII with a compound of Formula V to give a compound of Formula XVIII; and
reacting the compound of Formula XIII with an alkali metal methoxide to give the compound of Formula XX.

18. A method of making a compound of Formula XXII, 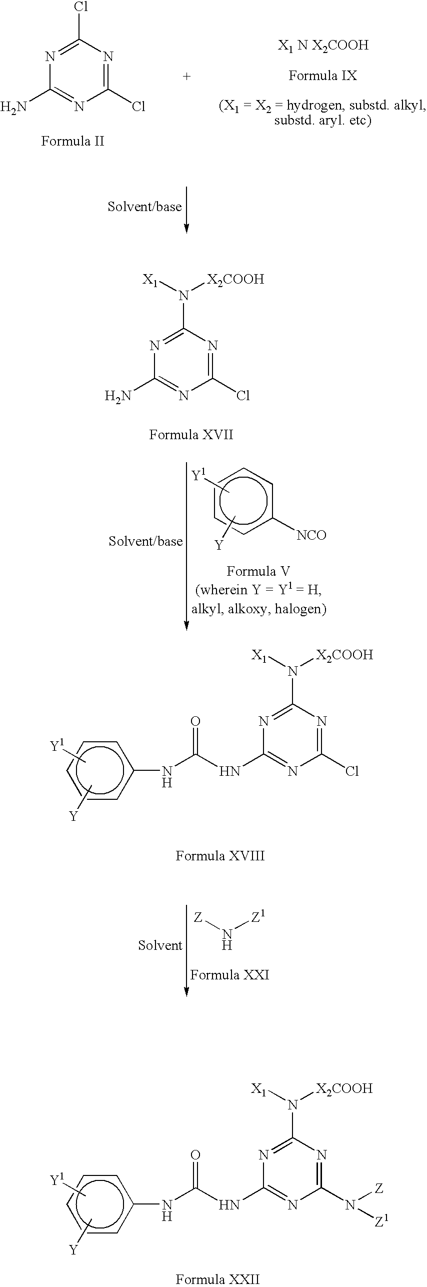
wherein Y and Y′ are independently hydrogen, alkyl, alkoxy, or halogen, X₁ and X₂ are independently hydrogen, substituted alkyl or substituted aryl, and Z and Z¹ are selected from hydrogen, substituted alkyl and substituted aryl, the method comprising:
reacting a compound of Formula II with a compound of Formula IX to give a compound of Formula XVII;
reacting the compound of Formula XVII with a compound of Formula V to give a compound of Formula XVIII; and
reacting the compound of Formula XIII with a compound of Formula XXI to give the compound of Formula XXII.
